(19) United States
(12) Patent Application Publication
(10) Pub. No.: US 2025/0332297 A1
(43) Pub. Date: Oct. 30, 2025

(54) DLL3-SPECIFIC BINDING CONSTRUCTS AND THEIR USE IN RADIOTHERAPY

(71) Applicants: MOLECULAR PARTNERS AG, Schlieren (CH); ORANO MED THERANOSTICS, Châtillon (FR)

(72) Inventors: Christian REICHEN, Schlieren (CH); Francesca MALVEZZI, Schlieren (CH); Amelie CROSET, Schlieren (CH); Stefanie RIESENBERG, Schlieren (CH); Amal SAIDI, Châtillon (FR); Aaron SCHATZMANN, Châtillon (FR); Julien TORGUE, Châtillon (FR)

(21) Appl. No.: 19/040,987

(22) Filed: Jan. 30, 2025

Related U.S. Application Data
(60) Provisional application No. 63/627,705, filed on Jan. 31, 2024.
(60) Provisional application No. 63/550,951, filed on Feb. 7, 2024.

(30) Foreign Application Priority Data
May 14, 2024 (EP) 24305751
Sep. 25, 2024 (EP) 24306567
Oct. 21, 2024 (EP) 24306765

(51) Int. Cl. A61K 51/08; A61K 51/04; A61P 35/00

(52) U.S. Cl. CPC A61K 51/08; A61K 51/0474; A61P 35/00

(57) ABSTRACT

The present invention relates to DLL3-specific binding constructs comprising a designed ankyrin repeat domain with binding specificity for DLL3, a connector, and a chelator capable of bonding to a radionuclide, such as Pb-212, as well as to such DLL3-specific binding constructs comprising a half-life extending moiety with binding specificity for serum albumin. The invention further relates to methods of producing such radio-labelled DLL3-specific binding constructs, pharmaceutical compositions comprising such constructs, and the use of such constructs or pharmaceutical compositions in methods for treating, imaging or diagnosing diseases, such as cancer.

### CROSS-REFERENCE TO RELATED APPLICATION

The present application claims the benefit of priority to U.S. 63/627,705, filed on Jan. 31, 2024; U.S. 63/550,951, filed on Feb. 7, 2024; EP 24305751, filed on May 14, 2024; EP 24306567, filed on Sep. 25, 2024; and EP 24306765, filed on Oct. 21, 2024. The disclosures of these patent applications are incorporated herein for all purposes by reference in their entirety.

### SEQUENCE LISTING

The instant application contains a Sequence Listing which has been submitted electronically in XML file format and is hereby incorporated by reference in its entirety. Said XML copy, created on Jan. 27, 2025, is named sequencelisting.txt.xml and is 36,084 bytes in size.

### FIELD

The present invention relates to DLL3-specific binding constructs comprising a designed ankyrin repeat domain with binding specificity for DLL3, a connector, and a chelator capable of bonding to a radionuclide, such as Pb-212, as well as to such DLL3-specific binding constructs comprising a half-life extending moiety with binding specificity for serum albumin. The invention further relates to methods of producing such radio-labelled DLL3-specific binding constructs, pharmaceutical compositions comprising such constructs, and the use of such constructs or pharmaceutical compositions in methods for treating, imaging or diagnosing diseases, such as cancer.

### BACKGROUND

Delta-like ligand 3 (DLL3) is an inhibitory protein of the Notch signaling system. DLL3 is expressed on the cell surface of various types of tumor cells, including small cell lung cancer (SCLC) and other high-grade endocrine tumors, but it is not expressed (or at a much lower level) on the cell surface in normal tissues. Therefore, DLL3 is considered a promising target for cancer therapy and diagnosis. Different types of DLL3 specific therapeutics have been developed and explored in clinical trials for cancer treatment, including anti-DLL3 bispecific T-cell engagers, CAR T cells and antibody-drug conjugates (Xiu M X et al, Onco Targets Ther, 13:3881-3901(2020)). However, some of these clinical programs were discontinued, mainly due to adverse effects such as increased gastrointestinal and cardiovascular toxicity, or due to lack of efficacy (Blackhall F et al, Journal of Thoracic Oncology; 16(9): 1547-1558 (2021); Yao J et al, The Oncologist; 27, 940-951; (2022)). Taken together, there remains a need for new DLL3-specific therapeutic and/or diagnostic agents and their use in treating and/or diagnosing diseases, such as cancer.

### SUMMARY OF THE INVENTION

Designing targeted radioisotope delivering platforms, including for alpha-particle emitting, beta-particle emitting or Auger electron emitting radioisotopes, and/or related drug candidates, requires simultaneous optimization of multiple aspects of such platforms or drug candidates. These aspects include, e.g., stability, target specificity, serum half-life, biodistribution, tissue penetration, pharmacodynamic properties, ease of manufacturing, acceptable therapeutic window and/or immunogenicity.

As an example, despite the excellent specificity of antibodies, such as IgGs, to their antigens, which makes antibodies an outstanding targeting platform for therapeutics, the typical serum half-life of an IgG of at least three weeks is disadvantageous for the delivery of radioisotopes, including alpha-emitting isotopes such as actinium-225 (225Ac) or lead-212 (212Pb) and beta-emitting isotopes such as lutetium-177 (177Lu) and yttrium-90 (90Y), in particular due to prolonged exposure and chronic off-target toxicities. Smaller antibody formats (e.g. monomeric scFv's, heavy-chain only antibodies, or single-domain antibody fragments) with a molecular weight of, e.g., 15 to 30 kDa have been engineered, which provide similarly good specificity as a full-size antibody, such as an IgG (about 150 kDa), but have a much shorter serum half-life (e.g. 30 minutes to 2 hours). However, such short half-lives do not provide sufficient time for efficacious target binding due to poor retention and tumor uptake, and furthermore plasma clearance of such small antibody formats by the renal system can lead to isotope accumulation in renal tissues and problematic off target toxicities.

Thus, despite the general potential of targeted radioisotope delivering therapy, further elucidation of biochemical, immunological, pharmacological, and molecular aspects of targeted radioisotope delivering platforms must be pursued to better design and develop effective targeted radioisotope delivering drug candidates. In this pursuit, various aspects may play a role, including the choice of the target antigen, of the target-specific delivery system, of the radionuclide payload, of the chelator used to bind the radionuclide payload, of the chemistry used to connect the chelator to the delivery system, and/or of the molecular mechanism or entity used to modulate pharmacokinetic properties.

Applicant has found that designed ankyrin repeat proteins (DARPins) with binding specificity for DLL3 can be formatted into targeted radioisotope delivering conjugates with beneficial properties. Such DARPin-based radioisotope delivering conjugates targeting DLL3, and methods of using such conjugates, are disclosed herein.

Based on the disclosure provided herein, those skilled in the art will recognize, or be able to ascertain using no more than routine experimentation, many equivalents to the specific embodiments of the invention described herein. Such equivalents are intended to be encompassed by the following embodiments (E).
E1. A conjugate or pharmaceutically acceptable salt thereof, the conjugate comprising (i) an ankyrin repeat domain with binding specificity for DLL3, (ii) a chelator, and (iii) a radionuclide, wherein said chelator is covalently connected to said ankyrin repeat domain, wherein said radionuclide is bound to said chelator, and wherein said radionuclide is Pb-212 or Pb-203.
E1a. The conjugate or salt of E1, wherein said conjugate has the formula: D-Ch-R, wherein D is said ankyrin repeat domain with binding specificity for DLL3, Ch is said chelator, and R is said radionuclide.
E1b. A conjugate or pharmaceutically acceptable salt thereof, the conjugate comprising (i) an ankyrin repeat domain with binding specificity for DLL3, (ii) a connector, (iii) a chelator, and (iv) a radionuclide, wherein said connector is covalently connected to said ankyrin repeat domain, wherein said chelator is covalently connected to said connector, wherein said radionuclide is bound to said chelator, and wherein said radionuclide is Pb-212 or Pb-203.
E2. The conjugate or salt of E1b, wherein said conjugate has the formula: D-Co-Ch-R, wherein D is said ankyrin repeat domain with binding specificity for DLL3, Co is said connector, Ch is said chelator, and R is said radionuclide.
E3. The conjugate or salt of any one of E1 to E2, wherein said ankyrin repeat domain with binding specificity for DLL3 binds human DLL3 with a K_{D} value of or below 100 nM, of or below 10 nM, of or below 3.5 nM, of or below 1 nM, of or below 350 pM, of or below 100 pM, of or below 35 pM, or of or below 10 pM.
E3a. The conjugate or salt of any one of E1 to E3, wherein said ankyrin repeat domain with binding specificity for DLL3 has a melting temperature (Tm) of at least about 70° C., at least about 75° C., at least about 80° C., at least about 82° C., at least about 85° C., at least about 88° C., or at least about 90° C.
E4. The conjugate or salt of any one of E1 to E3a, wherein said ankyrin repeat domain with binding specificity for DLL3 comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 1 to 4.
E5. The conjugate or salt of any one of E1 to E4, wherein said ankyrin repeat domain with binding specificity for DLL3 comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 1.
E6. The conjugate or salt of any one of E1 to E4, wherein said ankyrin repeat domain with binding specificity for DLL3 comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 2.
E7. The conjugate or salt of any one of E1 to E4, wherein said ankyrin repeat domain with binding specificity for DLL3 comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 3.
E8. The conjugate or salt of any one of E1 to E4, wherein said ankyrin repeat domain with binding specificity for DLL3 comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 4.
E9. The conjugate or salt of any one E1 to E8, wherein said chelator is DOTA (1,4,7,10-tetraazacyclododecane-1,4,7,10-tetraacetic acid) or TCMC (1,4,7,10-Tetraazacyclododecane-1,4,7,10-tetraacetamide), or a derivative thereof.
E10. The conjugate or salt of any one of E1 to E9, wherein said chelator has a structure of Formula (I):

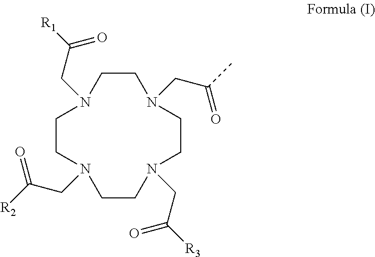

wherein R1, R2 and R3 are independently NH₂ or OH, and wherein the dotted line represents the covalent connection to said ankyrin repeat domain with binding specificity for DLL3 or said connector.
E11. The conjugate or salt of any one of E1 to E10, wherein said chelator has a structure of Formula (II):

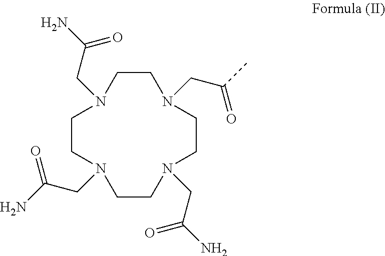

wherein the dotted line represents the covalent connection to said ankyrin repeat domain with binding specificity for DLL3 or said connector.

E12. The conjugate or salt of any one E1 to E9, wherein said chelator has a structure of Formula (III):

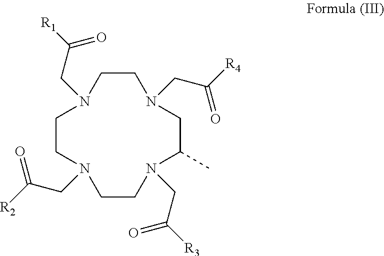

wherein R1, R2, R3 and R4 are independently NH₂ or OH, and wherein the dotted line represents the covalent connection to said ankyrin repeat domain with binding specificity for DLL3 or said connector.
E13. The conjugate or salt of any one of E1 to E9 and E12, wherein said chelator has a structure of Formula (IV):

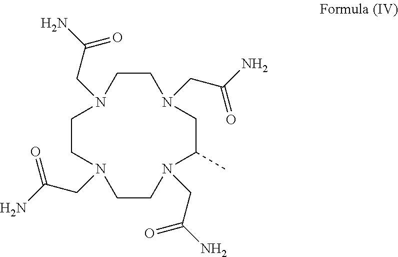 [SMILES: C[C@H]1CN(CC(N)=O)CCN(CC(N)=O)CCN(CC(N)=O)CCN1CC(N)=O]

wherein the dotted line represents the covalent connection to said ankyrin repeat domain with binding specificity for DLL3 or said connector.
E14. The conjugate or salt of any one of E1 to E13, further comprising a tag, wherein said tag comprises a Cysteine.
E15. The conjugate or salt of E14, wherein said tag is located at the C-terminal side of said ankyrin repeat domain with binding specificity for DLL3.
E16. The conjugate or salt of any one of E14 to E15, wherein said conjugate has the formula: D-T-Co-Ch-R, wherein D is said ankyrin repeat domain with binding specificity for DLL3, T is said tag, Co is said connector, Ch is said chelator, and R is said radionuclide.
E17. The conjugate or salt of any one of E14 to E16, wherein said tag comprises the amino acid sequence of SEQ ID NO: 10 or a variant thereof.
E18. The conjugate or salt of any one of E14 to E17, wherein said connector is covalently bound to said tag via a thioether bond.
E19. The conjugate or salt of any one of E1b to E18, wherein said connector comprises a maleimide or a derivative thereof.
E20. The conjugate or salt of any one of E1 to E19, wherein said connector has a structure of Formula (V):

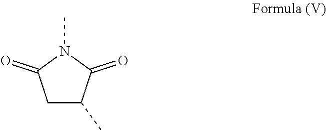

wherein the dotted line originating from N represents the covalent connection to said chelator, and wherein the dotted line originating from a carbon atom represents the covalent connection to said ankyrin repeat domain with binding specificity for DLL3 or said tag.
E21. The conjugate or salt of any one of E1 to E20, further comprising a half-life extending moiety.
E21a. The conjugate or salt of E21, wherein said conjugate has the formula: H-D-Ch-R, wherein H is said half-life extending moiety, D is said ankyrin repeat domain with binding specificity for DLL3, Ch is said chelator, and R is said radionuclide.
E21b. The conjugate or salt of E21, wherein said conjugate has the formula: D-H-Ch-R, wherein H is said half-life extending moiety, D is said ankyrin repeat domain with binding specificity for DLL3, Ch is said chelator, and R is said radionuclide.
E22. The conjugate or salt of E21, wherein said conjugate has the formula: H-D-Co-Ch-R, wherein H is said half-life extending moiety, D is said ankyrin repeat domain with binding specificity for DLL3, Co is said connector, Ch is said chelator, and R is said radionuclide.
E23. The conjugate or salt of E21, wherein said conjugate has the formula: H-D-T-Co-Ch-R, wherein H is said half-life extending moiety, D is said ankyrin repeat domain with binding specificity for DLL3, T is said tag, Co is said connector, Ch is said chelator, and R is said radionuclide.
E24. The conjugate or salt of E21, wherein said conjugate has the formula: D-H-Co-Ch-R, wherein H is said half-life extending moiety, D is said ankyrin repeat domain with binding specificity for DLL3, Co is said connector, Ch is said chelator, and R is said radionuclide.
E25. The conjugate or salt of E21, wherein said conjugate has the formula: D-H-T-Co-Ch-R, wherein H is said half-life extending moiety, D is said ankyrin repeat domain with binding specificity for DLL3, T is said tag, Co is said connector, Ch is said chelator, and R is said radionuclide.
E25a. The conjugate or salt of any one of E21 to E25, wherein said half-life extending moiety has binding specificity for human serum albumin.
E26. The conjugate or salt of any one of E21 to E25a, wherein said half-life extending moiety is an ankyrin repeat domain with binding specificity for human serum albumin.
E26a. The conjugate or salt of E26, wherein said ankyrin repeat domain with binding specificity for human serum albumin is covalently connected to said ankyrin repeat domain with binding specificity for DLL3 at the N-terminal side of said ankyrin repeat domain with binding specificity for DLL3.
E27. The conjugate or salt of any one of E26 to E26a, wherein said ankyrin repeat domain with binding specificity for human serum albumin binds human serum albumin with a K_{D} value of or below 500 nM, of or below 250 nM, or of or below 100 nM.
E28. The conjugate or salt of any one of E26 to E27, wherein said ankyrin repeat domain with binding specificity for human serum albumin comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 5 to 7.
E29. The conjugate or salt of any one of E26 to E28, wherein said ankyrin repeat domain with binding specificity for human serum albumin comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 5.
E30. The conjugate or salt of any one of E26 to E28, wherein said ankyrin repeat domain with binding specificity for human serum albumin comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 6.
E31. The conjugate or salt of any one of E26 to E28, wherein said ankyrin repeat domain with binding specificity for human serum albumin comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 7.
E31a. The conjugate or salt of any one of E26 to E31, wherein said tag is located at the C-terminal side of said ankyrin repeat domain with binding specificity for DLL3.
E32. The conjugate or salt of any one of E26 to E31a, wherein said ankyrin repeat domain with binding specificity for human serum albumin is covalently connected to said ankyrin repeat domain with binding specificity for DLL3 by a peptide linker.
E33. The conjugate or salt of E32, wherein said peptide linker comprises the amino acid sequence of any one of SEQ ID NOs: 8 or 9, or a variant thereof.
E34. The conjugate or salt of any one of E32 to E33, wherein said peptide linker comprises the amino acid sequence of SEQ ID NO: 8 or a variant thereof.
E35. The conjugate or salt of any one of E32 to E34, wherein said conjugate has the formula: H-L-D-Co-Ch-R, wherein H is said half-life extending moiety, L is said peptide linker, D is said ankyrin repeat domain with binding specificity for DLL3, Co is said connector, Ch is said chelator, and R is said radionuclide.
E36. The conjugate or salt of any of E32 to E34, wherein said conjugate has the formula: H-L-D-T-Co-Ch-R, wherein H is said half-life extending moiety, L is said peptide linker, D is said ankyrin repeat domain with binding specificity for DLL3, T is said tag, Co is said connector, Ch is said chelator, and R is said radionuclide.
E37. The conjugate or salt of any one of E32 to E34, wherein said conjugate has the formula: D-L-H-Co-Ch-R, wherein H is said half-life extending moiety, L is said peptide linker, D is said ankyrin repeat domain with binding specificity for DLL3, Co is said connector, Ch is said chelator, and R is said radionuclide.
E38. The conjugate or salt of any one of E32 to E34, wherein said conjugate has the formula: D-L-H-T-Co-Ch-R, wherein H is said half-life extending moiety, L is said peptide linker, D is said ankyrin repeat domain with binding specificity for DLL3, T is said tag, Co is said connector, Ch is said chelator, and R is said radionuclide.
E39. The conjugate or salt of any one of E1 to E38, wherein said conjugate comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 11 to 14.
E40. The conjugate or salt of any one of E1 to E39, wherein said conjugate or salt comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 11.
E41. The conjugate or salt of any one of E1 to E39, wherein said conjugate or salt comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 12.
E42. The conjugate or salt of any one of E1 to E39, wherein said conjugate or salt comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 13.
E43. The conjugate or salt of any one of E1 to E39, wherein said conjugate or salt comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 14.
E44. The conjugate or salt of any one of E39 to E43, wherein said amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 11 to 14 is covalently bound at its N-terminal end to Glycine-Serine (GS).
E45. The conjugate or salt of any one of E39 to E44, wherein said amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 11 to 14 is covalently bound at its C-terminal end to the amino acid sequence of SEQ ID NO: 10.
E46. A conjugate or pharmaceutically acceptable salt thereof, the conjugate comprising (i) an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 15 to 18, (ii) a chelator, and (iii) a radionuclide, wherein said ankyrin repeat protein has binding specificity for DLL3 and for human serum albumin, wherein said chelator is covalently connected to said ankyrin repeat protein, wherein said radionuclide is bound to said chelator, and wherein said radionuclide is Pb-212 or Pb-203.
E46a. A conjugate or pharmaceutically acceptable salt thereof, the conjugate comprising (i) an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 15 to 18, (ii) a connector, (iii) a chelator, and (iv) a radionuclide, wherein said ankyrin repeat protein has binding specificity for DLL3 and for human serum albumin, wherein said connector is covalently connected to said ankyrin repeat protein, wherein said chelator is covalently connected to said connector, wherein said radionuclide is bound to said chelator, and wherein said radionuclide is Pb-212 or Pb-203.
E47. The conjugate or salt of any one of E46 to E46a, wherein said conjugate comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 15.
E48. The conjugate or salt of any one of E46 to E46a, wherein said conjugate comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 16.
E49. The conjugate or salt of any one of E46 to E46a, wherein said conjugate comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 17.
E50. The conjugate or salt of any one of E46 to E46a, wherein said conjugate comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 18.
E51. The conjugate or salt of any one of E46a to E50, wherein said ankyrin repeat protein comprises a Cysteine, wherein said connector comprises maleimide or a derivative thereof, and wherein said Cysteine is covalently bound to said connector via a thioether bond.
E52. The conjugate or salt of E51, wherein said Cysteine is located at the C-terminal end of said amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 15 to 18.
E52a. The conjugate or salt of any one of E46 to E52, wherein said ankyrin repeat protein has a melting temperature (Tm) of at least about 70° C., at least about 75° C., at least about 80° C., at least about 82° C., at least about 85° C., at least about 88° C., or at least about 90° C.
E53. The conjugate or salt of any one of E46 to E52a, wherein said chelator is DOTA (1,4,7,10-tetraazacyclododecane-1,4,7,10-tetraacetic acid) or TCMC (1,4,7,10-Tetraazacyclododecane-1,4,7,10-tetraacetamide), or a derivative thereof.
E54. The conjugate or salt of any one of E46 to E53, wherein said chelator has a structure of Formula (I):

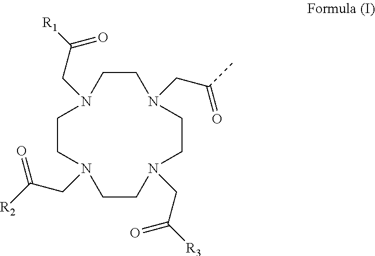

wherein R1, R2 and R3 are independently NH₂ or OH, and wherein the dotted line represents the covalent connection to said ankyrin repeat protein or said connector.
E55. The conjugate or salt of any one of E46 to E53, wherein said chelator has a structure of Formula (II):

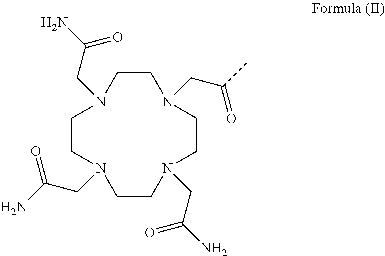

wherein the dotted line represents the covalent connection to said ankyrin repeat protein or said connector.
E56. The conjugate or salt of any one of E46 to E53, wherein said chelator has a structure of Formula (III):

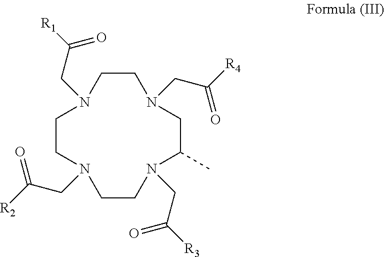

wherein R1, R2, R3 and R4 are independently NH₂ or OH, and wherein the dotted line represents the covalent connection to said ankyrin repeat protein or said connector.
E57. The conjugate or salt of any one of E46 to E53, wherein said chelator is has a structure of Formula (IV):

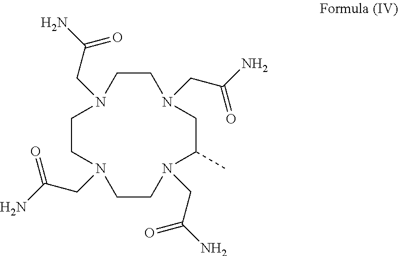 [SMILES: C[C@H]1CN(CC(N)=O)CCN(CC(N)=O)CCN(CC(N)=O)CCN1CC(N)=O]

wherein the dotted line represents the covalent connection to said ankyrin repeat protein or said connector.
E58. A conjugate or pharmaceutically acceptable salt thereof, wherein said conjugate has a structure of Formula (VI):

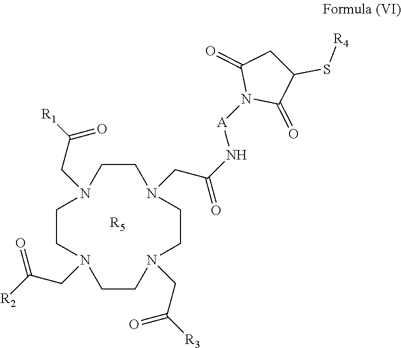

wherein R1, R2, and R3 are independently NH₂ or OH;
wherein A is CₐH_{b}N_{c}Oₐ, wherein a, b, c, and d are integers;
wherein R4 is an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 15 to 18, wherein said ankyrin repeat protein has binding specificity for DLL3 and for human serum albumin, and wherein said ankyrin repeat protein comprises a Cysteine;
and wherein R5 is a chelated radionuclide, wherein said radionuclide is Pb-212 or Pb-203.
E58a. The conjugate or salt of E58, wherein said Cysteine is connected to a heterocyclic ring structure by a thioether bond, wherein said heterocyclic ring structure connects A and R4.
E58b. The conjugate or salt of any one of E58 to E58a, wherein R1, R2 and R3 are NH₂.
E58c. The conjugate or salt of any one of E58 to E58b, wherein A is a hydrocarbon bridge.
E58d. The conjugate or salt of any one of E58 to E58c, wherein A is—CH2-CH2-.
E58e. A conjugate or pharmaceutically acceptable salt thereof, wherein said conjugate comprises 1,4,7,10-Tetraazacyclododecane-1,4,7-tris(carbamoylmethyl)-10-maleimidoethylacetamide, an ankyrin repeat protein, and a radionuclide, wherein said ankyrin repeat protein comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 15 to 18, wherein said ankyrin repeat protein has binding specificity for DLL3 and for human serum albumin, wherein said ankyrin repeat protein comprises a Cysteine, wherein said ankyrin repeat protein is connected to said 1,4,7,10-Tetraazacyclododecane-1,4,7-tris(carbamoylmethyl)-10-maleimidoethylacetamide via a thioether bond formed between said Cysteine and the maleimide ring of said 1,4,7,10-Tetraazacyclododecane-1,4,7-tris(carbamoylmethyl)-10-maleimidoethylacetamide, wherein said radionuclide is Pb-212 or Pb-203, and wherein said radionuclide is coordinated by said 1,4,7,10-Tetraazacyclododecane-1,4,7-tris(carbamoylmethyl)-10-maleimidoethylacetamide.
E59. A conjugate or pharmaceutically acceptable salt thereof, wherein said conjugate has a structure of Formula (VII):

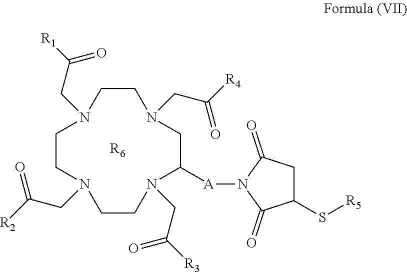

wherein R1, R2, R3 and R4 are independently NH₂ or OH;
wherein A is CₐH_{b}N_{c}O_{d}, wherein a, b, c, and d are integers;
wherein R5 is an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 15 to 18, wherein said ankyrin repeat protein has binding specificity for DLL3 and for human serum albumin, and wherein said ankyrin repeat protein comprises a Cysteine;
and wherein R6 is a chelated radionuclide, wherein said radionuclide is Pb-212 or Pb-203.
E59a. The conjugate or salt of E59, wherein said Cysteine is connected to a heterocyclic ring structure by a thioether bond, wherein said heterocyclic ring structure connects A and R5.
E59b. The conjugate or salt of any one of E59 to E59a, wherein R1, R2, R3 and R4 are NH₂.
E59c. The conjugate or salt of any one of E59 to E59b, wherein A is a hydrocarbon bridge.
E59d. The conjugate or salt of any one of E59 to E59c, wherein A is—CH2-CH2-.
E60. The conjugate or salt of any one of E58 to E59d, wherein said conjugate or salt comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 15.
E61. The conjugate or salt of any one of E58 to E59d, wherein said conjugate or salt comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 16.
E62. The conjugate or salt of any one of E58 to E59d, wherein said conjugate or salt comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 17.
E63. The conjugate or salt of any one of E58 to E59d, wherein said conjugate or salt comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 18.
E64. The conjugate or salt of any one of E58 to E63, wherein said Cysteine is located at the C-terminal end of said amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 15 to 18.
E65. The conjugate or salt of any one of E46 to E64, wherein said ankyrin repeat protein binds human DLL3 with a K_{D} value of or below 100 nM, of or below 10 nM, of or below 3.5 nM, of or below 1 nM, of or below 350 pM, of or below 100 pM, of or below 35 pM, or of or below 10 pM.
E66. The conjugate or salt of any one of E46 to E65, wherein said ankyrin repeat protein binds human serum albumin with a K_{D} value of or below 500 nM, of or below 250 nM, or of or below 100 nM.
E66a. The conjugate or salt of any one of E1 to E66, wherein said conjugate binds to cells expressing human DLL3 on their surface.
E66b. The conjugate or salt of any one of E1 to E66a, wherein said conjugate binds to cells expressing human DLL3 on their surface, and wherein said cells are HEK293T cells engineered to express human DLL3 on their surface.
E66c. The conjugate or salt of E66b, wherein said conjugate binds said HEK293T cells engineered to express human DLL3 on their surface with an EC50 below 10⁻⁷M, or about or below 5×10⁻⁸M, or about or below 2×10⁻⁸M, or about or below 10⁻⁸M, or about or below 5×10⁻⁹M, or about or below 10⁻⁹M.
E66d. The conjugate or salt of any one of E1 to E66c, wherein said conjugate binds to cells expressing human DLL3 on their surface, and wherein said cells are NCI-H82 cells.
E66e. The conjugate or salt of E66d, wherein said conjugate binds said NCI-H82 cells with an EC50 below 10⁻⁷M, or about or below 3×10⁻⁸M, or about or below 10⁻⁸M, or about or below 6×10⁻⁹M, or about or below 3×10⁻⁹M, or about or below 10⁻⁹M.
E66f. The conjugate or salt of any one of E1 to E66e, wherein said conjugate has a terminal half-life in a mouse model of at least about 5 hours, at least about 8 hours, at least about 10 hours, at least about 12 hours, at least about 15 hours, at least about 18 hours, at least about 20 hours, at least about 23 hours, at least about 25 hours, at least about 28 hours, at least about 30 hours, at least about 33 hours, at least about 35 hours, at least about 38 hours, or at least about 40 hours.
E66g. The conjugate or salt of any one of E1 to E66f, wherein said conjugate has a terminal half-life in a mouse model of about 5 to 45 hours, about 5 to 40 hours, about 5 to 35 hours, about 10 to 35 hours, about 20 to 35 hours, about 25 to 35 hours, about 25 to 40 hours, about 30 to 40 hours, or about 30 to 35 hours.
E66h. The conjugate or salt of any one of E66f and E66g, wherein said mouse model is a BALB/c mouse model.
E66i. The conjugate or salt of any one of E1 to E66h, wherein said radionuclide is Pb-212, and wherein said conjugate is capable of inhibiting tumor growth in a human DLL3-expressing mouse tumor model.
E66j. The conjugate or salt of E66i, wherein said human DLL3-expressing mouse tumor model comprises tumors formed by human DLL3-expressing MC38 colon carcinoma cells or by NCI-H82 lung carcinoma cells.
E66k. The conjugate or salt of any one of E1 to E66j, wherein said conjugate reaches a tumor-to-kidney ratio (T:K) in a human DLL3-expressing mouse tumor model at 24 hours after administration of said conjugate of at least about 1.0, at least about 1.2, at least about 1.5, at least about 1.7, at least about 2.0, at least about 2.2, or at least about 2.5.
E66l. The conjugate or salt of E66k, wherein said tumor-to-kidney ratio (T:K) is determined in a human DLL3-expressing MC38 colon carcinoma cell mouse tumor model or in a NCI-H82 lung carcinoma cell mouse tumor model.
E67. A pharmaceutical composition comprising the conjugate or salt of any one of E1 to E66l, and optionally a pharmaceutically acceptable carrier or excipient.
E68. A kit comprising (i) a first container containing the conjugate or salt of any one of E1 to E66l or the pharmaceutical composition of E67; and (ii) a second container containing a buffered solution.
E69. The conjugate or salt of any of E1 to E66l or the pharmaceutical composition of E67 for use in imaging, diagnosing and/or treating a medical condition.
E69a. The conjugate or salt of any of E1 to E66l or the pharmaceutical composition of E67 for use in a method of imaging, diagnosing and/or treating a medical condition, the method comprising the step of administering to a subject in need thereof an amount of said conjugate or salt or said pharmaceutical composition effective for imaging, diagnosing and/or treating said medical condition.
E70. The conjugate or salt or the pharmaceutical composition for use in imaging, diagnosing and/or treating a medical condition of E69 or for use in a method of imaging, diagnosing and/or treating a medical condition of E69a, wherein said medical condition is cancer.
E71. The conjugate or salt or the pharmaceutical composition for use in imaging, diagnosing and/or treating a medical condition or for use in a method of imaging, diagnosing and/or treating a medical condition of E70, wherein said cancer comprises cells that express DLL3 on their surface.
E72. The conjugate or salt or the pharmaceutical composition for use in imaging, diagnosing and/or treating a medical condition or for use in a method of imaging, diagnosing and/or treating a medical condition of any one of E70 to E71, wherein said cancer is a neuroendocrine cancer.
E73. The conjugate or salt or the pharmaceutical composition for use in imaging, diagnosing and/or treating a medical condition or for use in a method of imaging, diagnosing and/or treating a medical condition of any one of E70 to E72, wherein said cancer is glioma, neuroendocrine lung cancer, neuroendocrine prostate cancer, gastrointestinal neuroendocrine cancer or small cell bladder cancer.
E74. The conjugate or salt or the pharmaceutical composition for use in imaging, diagnosing and/or treating a medical condition or for use in a method of imaging, diagnosing and/or treating a medical condition of any one of E70 to E73, wherein said cancer is small cell lung cancer.
E75. The conjugate or salt or the pharmaceutical composition for use in imaging, diagnosing and/or treating a medical condition or for use in a method of imaging, diagnosing and/or treating a medical condition of any one of E70 to E74, wherein said cancer is small cell lung carcinoma.
E76. The conjugate or salt or the pharmaceutical composition for use in imaging, diagnosing and/or treating a medical condition or for use in a method of imaging, diagnosing and/or treating a medical condition of any one of E69 to E75, wherein said subject is a mammal, preferably a human.
E77. The conjugate or salt or the pharmaceutical composition for use in imaging, diagnosing and/or treating a medical condition or for use in a method of imaging, diagnosing and/or treating a medical condition of any one of E69 to E76, wherein said radionuclide is Pb-212.
E78. The conjugate or salt or the pharmaceutical composition for use in imaging, diagnosing and/or treating a medical condition or for use in a method of imaging, diagnosing and/or treating a medical condition of any one of E69 to E76, wherein said radionuclide is Pb-203.
E79. A method of treating a medical condition, the method comprising the step of administering to a subject in need thereof a therapeutically effective amount of the conjugate or salt of any one of E1 to E66l or the pharmaceutical composition of E67.
E80. The method of E79, wherein said radionuclide is Pb-212.
E81. A method of imaging and/or diagnosing a medical condition, the method comprising the steps of: (i) administering to a subject an amount of the conjugate or salt of any one of E1 to E66l, or of the pharmaceutical composition of E67, effective for binding of the conjugate or salt to cells expressing DLL3 on their surface, and (ii) detecting cells bound by the conjugate or salt thereof or tissues comprising cells bound by the conjugate or salt thereof.
E82. The method of E81, wherein said detecting in step (ii) is performed by in vivo imaging.
E83. The method of E82, wherein said in vivo imaging uses single photon emission computed tomography (SPECT).
E84. The method of any one of E81 to E83, wherein said radionuclide is Pb-203.
E85. The method of any one of E79 to E84, wherein said medical condition is cancer.
E86. The method of E85, wherein said cancer comprises cells that express DLL3 on their surface.
E87. The method of any one of E85 to E86, wherein said cancer is a neuroendocrine cancer.
E88. The method of any one of E85 to E87, wherein said cancer is glioma, neuroendocrine lung cancer, neuroendocrine prostate cancer, gastrointestinal neuroendocrine cancer or small cell bladder cancer.
E89. The method of any one of E85 to E88, wherein said cancer is small cell lung cancer.
E90. The method of any one of E85 to E89, wherein said cancer is small cell lung carcinoma.
E91. The method of any one of E79 to E90, wherein said subject is a mammal, preferably a human.
E92. The conjugate or salt of any of E1 to E66l or the pharmaceutical composition of E67 for use in a method of manufacturing a medicament.
E93. The conjugate or salt or the pharmaceutical composition for use in a method of manufacturing a medicament of E92, wherein said medicament is for the treatment of cancer, optionally for the treatment of a neuroendocrine cancer, optionally for the treatment of small cell lung cancer.
E94. A method of manufacturing a medicament for the treatment of a medical condition, wherein the conjugate or salt of any one of E1 to E66l or the pharmaceutical composition of E67 is an active ingredient of said medicament.
E95. The method of manufacturing a medicament for the treatment of a medical condition of E94, wherein said medical condition is cancer, optionally a neuroendocrine cancer, optionally small cell lung cancer.

### BRIEF DESCRIPTION OF THE DRAWINGS

FIGS. 1A and 1B: Illustration of the structures of exemplary embodiments of the DLL3-specific conjugates disclosed herein. FIG. 1A: R1, R2, and R3 are independently NH₂ or OH; A is CₐH_{b}N_{c}O_{d}, wherein a, b, c, and d are integers; R4 is an ankyrin repeat protein comprising the amino acid sequence of any one of SEQ ID NOs: 15 to 18 or a variant thereof; R5 is a chelated radionuclide, either Pb-212 or Pb-203. FIG. 1A shows a structure of Formula (VI). FIG. 1B: R1, R2, R3 and R4 are independently NH₂ or OH; A is CₐH_{b}N_{c}O_{d}, wherein a, b, c, and d are integers; R5 is an ankyrin repeat protein comprising the amino acid sequence of any one of SEQ ID NOs: 15 to 18 or a variant thereof; R6 is a chelated radionuclide, either Pb-212 or Pb-203. FIG. 1B shows a structure of Formula (VII).

FIGS. 2A and 2B: In vivo biodistribution of Pb-212 labelled DARPin (MAM120, MAM160) conjugates. Radio-labelled MAM120 and MAM160 conjugates were injected iv. at 0.01 mg/kg (10 μCi) into R2G2 mice xenografted subcutaneously with NCI-H82 cells. Blood, spleen, kidneys, liver and tumor were collected 4 h and 24h post injection and radioactivity of each sample was measured using a γ-counter instrument. FIG. 2A: MAM120 conjugate; FIG. 2B: MAM160 conjugate.

FIG. 3: In vivo biodistribution of Pb-212 labelled DARPin (MAM160) conjugate. Radio-labelled MAM160 conjugate was injected i.v. at 0.01 mg/kg (10 μCi) into athymic nude mice xenografted subcutaneously with hDLL3-MC38 cells. Blood, bladder, reproductive organs, small intestine, colon, spleen, pancreas, kidneys, stomach, liver, lung, heart, brain, femoral bone, abdominal fat, skeletal muscle, tail, and tumor were collected 1 h, 4 h and 24h post injection and radioactivity of each sample was measured using a γ-counter instrument.

FIG. 4: Efficacy of Pb-212 labelled DARPin (MAM160) and Rova conjugates. Radio-labelled molecules were injected i.v. at 0.01 mg/kg (10 μCi) into athymic nude mice xenografted subcutaneously with hDLL3-MC38 cells. Animals were under observation daily and 3× per week tumors were measured by caliper. Animals were sacrificed when tumors reached 1000 mm³ or if termination criteria were met. The data were expressed as average+/−SEM of tumor volume in mm³. SEM: standard error of the mean.

FIG. 5: Dose response finding profile of Pb-212 labelled DARPin (MAM160) conjugate. Radio-labelled MAM160 conjugate was injected i.v. at 10 μCi, 20 μCi, 30 μCi and 40 μCi into WT CD1 mice. Animals were under observation daily and 3× per week animals were weighed. Animals were sacrificed 3 weeks after the first injection or if termination criteria were met. The data were expressed as % of body weight (BW) change (relative to the initial BW at Day −7 before the treatment). SEM: standard error of the mean.

FIGS. 6A to 6C: In vivo biodistribution of Pb-212 labelled DARPin (MAM279, MAM283, MAM160) conjugates. Radio-labelled molecules were injected i.v. at 0.01 mg/kg (10 μCi) into athymic nude mice xenografted subcutaneously with hDLL3-MC38 cells. Blood, kidneys, liver, and tumor were collected 4 h and 24h post injection and radioactivity of each sample was measured using a β-counter instrument. FIG. 6A: MAM279 conjugate; FIG. 6B: MAM283 conjugate; FIG. 6C: MAM160 conjugate.

FIG. 7: In vivo biodistribution of Pb-212 labelled DARPin (MAM282) conjugate. Radio-labelled MAM282 conjugate was injected i.v. at 0.01 mg/kg (10 μCi) into athymic nude mice xenografted subcutaneously with hDLL3-MC38 cells. Blood, kidneys, liver, and tumor were collected 4 h and 24h post injection and radioactivity of each sample was measured using a β-counter instrument.

FIGS. 8A and 8B: In vivo biodistribution of Pb-212 labelled DARPin (MAM279) conjugate. Radio-labelled MAM279 conjugate was injected at 0.01 mg/kg (10 μCi) i.v. into R2G2 mice xenografted subcutaneously with NCI-H82 cells and into the tail vein of athymic nude mice xenografted subcutaneously with hDLL3-MC38 cells. Blood, bladder, small intestine, colon, spleen, kidneys, liver, lung, heart, tail and tumor were collected 4 h and 24h post injection and radioactivity of each sample was measured using a γ-counter instrument. FIG. 8A: hDLL3-MC38 tumor model; FIG. 8B: NCI-H82 tumor model.

FIGS. 9A to 9D: Binding of different concentrations of selected single domain (1 D) and two domain (2D) DARPins to HEK293T-hDLL3 cells expressing human DLL3, in absence and presence of 10 μM human serum albumin. FIG. 9A: 2D DARPins, without HSA; FIG. 9B: 2D DARPins, with HSA; FIG. 9C: 1D DARPins, without HSA; FIG. 9D: 1 D DARPins, with HSA.

FIGS. 10A and 10B: Binding of different concentrations of selected two domain (2D) DARPins to NCI-H82 cells expressing human DLL3, in absence and presence of 10 μM human serum albumin. A non-binding DARPin was used as a negative control. FIG. 10A: MAM279, MAM283, and control DARPin, with and without HSA; FIG. 10B: MAM282 and control DARPin, with and without HSA.

FIGS. 11A to 11D: Surface Plasmon Resonance (SPR) multi-trace analysis of 2D DARPin (MAM279), with or without conjugation to a chelator (DOTAM), binding to human DLL3 (extracellular domain (ECD) or only N-terminal domain (NTD)). Various concentrations of DARPins were applied to immobilized human DLL3 for on-rate and off-rate measurements. FIGS. 10A to 10D show representative SPR sensograms. FIG. 10A: binding of MAM279 to hDLL3-ECD; FIG. 10B: binding of DOTAM-conjugated MAM279 to hDLL3-ECD; FIG. 10C: binding of MAM279 to hDLL3-NTD; FIG. 10D: binding of DOTAM-conjugated MAM279 to hDLL3-NTD. RU, Resonance Units; s, time in seconds.

FIGS. 12A to 12C: Thermal stability assessment of 2D DARPins using Circular Dichroism (CD) spectroscopy. FIG. 11A: MAM279; FIG. 11B: MAM283; FIG. 11C: MAM282. Upper graphs: Overlay of spectra taken before and after the temperature scan. Lower graphs: Protein unfolding (forward) and refolding (reverse) monitored during the temperature gradient. MRE: mean residue ellipticity.

FIG. 13: Pharmacokinetic analysis of natural lead-labelled DARPin (MAM279, MAM283, MAM160, MAM282) conjugates in mice. DARPin conjugates were injected iv. at 1 mg/kg into WT BALBc mice. Serum was collected 2 min, 10 min, 30 min, 1 h, 4 h, 24 h, 48 h, 72 h post-injection. DARPin was detected and measured by ELISA. LLOQ: Lower Limit of Quantification.

FIG. 14: Pharmacokinetic analysis of natural lead-labelled DARPin (MAM279) conjugate at different doses in mice. The DARPin conjugate was injected iv. at 0.1 mg/kg or at 1 mg/kg into WT BALBc mice. Serum was collected 2 min, 10 min, 30 min, 1 h, 4 h, 24 h, 48 h, 72 h post-injection for MAM279 conjugate injected at 1 mg/kg, and 2 min, 30 min, 4 h, 24 h, 30 h, 48 h, 72 h, 96 h, 168 h post-injection for MAM279 conjugate injected at 0.1 mg/kg. DARPin was detected and measured by ELISA. LLOQ: Lower Limit of Quantification.

FIGS. 15A and 158: Dose response finding profiles of Pb-212 labelled DARPin (MAM279, MAM283) conjugates. Radio-labelled DARPin (MAM279 and MAM283) conjugates were injected iv. at 10 μCi, 20 μCi, 30 μCi and 60 μCi into WT CD1 mice. Animals were under observation daily and 3× per week animals were weighed. Animals were sacrificed 3 weeks after the first injection or if termination criteria were met. The data were expressed as % of body weight (BW) change (relative to the initial BW at Day −7 before the treatment). FIG. 15A: MAM279 conjugate; FIG. 15B: MAM283 conjugate. SEM: standard error of the mean.

FIG. 16: Efficacy of Pb-212 labelled DARPin (MAM279) and Rova conjugates. Radio-labelled molecules were injected i.v. at 0.01 mg/kg (10 μCi) into R2G2 mice xenografted subcutaneously with NCI-H82 cells. Animals were under observation daily and 3× per week tumors were measured by caliper. Animals were sacrificed when tumors reached 1500 to 2000 mm³ or if termination criteria were met. The data were expressed as average+/−SEM of tumor volume in mm³. SEM: standard error of the mean.

FIG. 17: Simultaneous binding of a 2D DARPin (MAM279) conjugate to DLL3 and serum albumin. Binding of a 2D DARPin (MAM279), conjugated to a chelator (DOTAM), to human DLL3 and human serum albumin (HSA) was determined by SPR multi-trace analysis. SPR sensorgrams show a first injection of MAM279-DOTAM (injection 1) to immobilized hDLL3-ECD, followed by ˜300 sec lag time and a second injection of HSA (injection 2) with a dissociation time of 900 sec. The respective controls, where only the DARPin conjugate (thick interrupted line), only HSA (thin continuous line) or only PBST (thin interrupted line) were injected, are overlayed to the trace with all reagents used (thick continuous line). RU, Resonance Units; s, time in seconds.

FIGS. 18A to 18F: Internalization of half-life extended DLL3-specific ankyrin repeat protein in cells expressing DLL3 on their surface. Internalization of 2D DARPin MAM279 in different cell lines was investigated and compared to the antibody Rova. Molecules were labelled with AF488 and the internalized signals were assessed using an anti-AF488-quencher that quenches the external fluorescence. The signals were normalized to non-binding controls. FIG. 18A: MAM279 on SHP-77 cells; FIG. 18B: MAM279 on NCI-H82 cells; FIG. 18C: MAM279 on MC38-hDLL3 cells; FIG. 18D: Rova on SHP-77 cells; FIG. 18E: Rova on NCI-H82 cells; FIG. 18F: Rova on MC38-hDLL3 cells;

FIGS. 19A and 19B: Pharmacokinetic analysis of natural lead-labelled DARPin (MAM279) conjugate injected at different doses in mice. DARPin (MAM279) conjugate was injected iv. at 0.001 mg/kg, 0.01 mg/kg, 0.1 mg/kg or 1 mg/kg into the tail vein of WT BALBc mice. Unconjugated Rova antibody (as a comparison) was injected iv. at 1 mg/kg into the tail vein of WT BALBc mice. Serum was collected 2 min, 10 min, 30 min, 1 h, 4 h, 24 h, 48 h, and 72 h post-injection for MAM279 conjugate at 1 mg/kg and 2 min, 30 min, 4 h, 24 h, 30 h, 48 h, 72 h, 96 h, and 168 h post-injection for MAM279 conjugate at 0.1 mg/kg, 0.01 mg/kg and 0.001 mg/kg and for Rova antibody. DARPin (FIG. 19A) and Rova (FIG. 19B) were detected and measured by ELISA. The data are from three separate studies. LLOQ: Lower Limit of Quantification.

FIGS. 20A to 20D: Efficacy of Pb-212 labelled DARPin (MAM279) conjugate at weekly repeat-dosing. Radio-labelled molecules (MAM279 conjugate or negative DARPin control conjugate) were injected i.v. four times weekly at 10 μCi into R2G2 mice xenografted subcutaneously with NCI-H82 cells. The vertical dotted lines indicate the four weekly injections, at days 14, 21, 28 and 35 post-tumor cell xenograft. Animals were under observation daily and 3× per week tumors were measured by caliper. Animals were sacrificed when tumors reached 1500 to 2000 mm³ or if termination criteria were met. In FIG. 20A, tumor growth curves for all conditions and animals are shown, with the data expressed as average+/−SEM of tumor volume in mm³. In FIGS. 20B to 20D, tumor growth curves for individual animals are shown, injected with buffer only (FIG. 20B), radio-labelled MAM279 conjugate (FIG. 20C), or radio-labelled control DARPin conjugate (FIG. 20D). The data were expressed as tumor volume in mm³. SEM: standard error of the mean.

### DETAILED DESCRIPTION OF THE INVENTION

Designed ankyrin repeat domains are structural units of designed ankyrin repeat proteins. Designed repeat protein libraries, including designed ankyrin repeat protein libraries (WO2002020565; Binz et al., Nat. Biotechnol. 22, 575-582, 2004; Stumpp et al., Drug Discov. Today 13, 695-701, 2008), can be used for the selection of target-specific designed repeat domains that bind to their target with high affinity. Such target-specific designed repeat domains in turn can be used as valuable components of recombinant binding proteins for the treatment and/or diagnosis of diseases.

Designed ankyrin repeat proteins are a class of binding molecules which have the potential to overcome limitations of monoclonal antibodies, hence allowing novel therapeutic and/or diagnostic approaches. Such ankyrin repeat proteins may comprise a single designed ankyrin repeat domain, or may comprise a combination of two, three, four, five or more designed ankyrin repeat domains with the same or different target specificities (Stumpp et al., Drug Discov. Today 13, 695-701, 2008; U.S. Pat. No. 9,458,211). Ankyrin repeat proteins comprising only a single designed ankyrin repeat domain are small proteins (14 kDa) which can be selected to bind a given target protein with high affinity and specificity. These characteristics, and the possibility of combining two, three, four, five or more designed ankyrin repeat domains in one protein, make designed ankyrin repeat proteins ideal agonistic, antagonistic and/or inhibitory drug candidates. Furthermore, such ankyrin repeat proteins can be engineered to carry various effector functions, e.g. cytotoxic agents or half-life extending agents, enabling completely new drug formats. Taken together, designed ankyrin repeat proteins are an example of the next generation of protein therapeutics with the potential to surpass existing antibody drugs.

The inventors of the present invention have found that designed ankyrin repeat domains with binding specificity for DLL3 can be covalently combined with other moieties to form DLL3-specific binding constructs that can be loaded with a radionuclide of the theranostic pair of radionuclides, lead-203 (²⁰³Pb or Pb-203, t_{1/2}=51.9 h) and lead-212 (²¹²Pb or Pb-212, t_{1/2}=10.6 h). These radio-labelled DLL3-specific DARPin conjugates have beneficial properties that make them useful for applications in imaging, diagnosing and/or treating medical conditions characterized by DLL3 expression on the surface of cells, such as certain cancers. The daughter nuclides (²¹²Bi and ²¹²Po) of ²¹²Pb undergo α-decay, and hence ²¹²Pb can be viewed as an in vivo generator of alpha-particles emitters. Higher linear-energy transfer of alpha-particles (compared to beta-particles) may result in an increased incidence of double-strand DNA breaks and improved localized cancer cell damage. The elementally-matched isotope ²⁰³Pb may be used as an imaging surrogate in place of the therapeutic radionuclide. Such use of ²⁰³Pb may allow for a pharmacologically-inactive determination of the pharmacokinetics and biodistribution of a targeted radiotherapy drug candidate in advance of treatment and the identification of patients who may benefit from treatment.

### Conjugates or Pharmaceutically Acceptable Salts Thereof

In one main aspect, the invention relates to a conjugate or pharmaceutically acceptable salt thereof, the conjugate comprising (i) an ankyrin repeat domain with binding specificity for DLL3, (ii) a chelator, and (iii) a radionuclide, wherein said chelator is covalently connected to said ankyrin repeat domain, wherein said radionuclide is bound to said chelator, and wherein said radionuclide is Pb-212 or Pb-203. In one embodiment, said radionuclide is Pb-212. In another embodiment, said radionuclide is Pb-203.

In another aspect, the invention relates to such a conjugate or pharmaceutically acceptable salt thereof, wherein said conjugate has the formula: D-Ch-R, wherein D is said ankyrin repeat domain with binding specificity for DLL3, Ch is said chelator, and R is said radionuclide. Different methods of covalently connecting a polypeptide to a chelator have been described (see, e.g., in Morais and Ma, Drug Discovery Today: Technologies|Antibody—drug Conjugates (ADC), Vol. 30, pp. 91-104, 2018; Tsuchikama and An, Protein Cell 2018, 9(1):33-46); Kang et al., Chem. Sci., 2021, 12, 13613).

In another aspect, the invention relates to such a conjugate or pharmaceutically acceptable salt thereof, wherein said conjugate further comprises a connector, wherein said connector is covalently connected to said ankyrin repeat domain with binding specificity for DLL3 and to said chelator. Thus, in one aspect, the invention relates to a conjugate or pharmaceutically acceptable salt thereof, the conjugate comprising (i) an ankyrin repeat domain with binding specificity for DLL3, (ii) a connector, (iii) a chelator, and (iv) a radionuclide, wherein said connector is covalently connected to said ankyrin repeat domain, wherein said chelator is covalently connected to said connector, wherein said radionuclide is bound to said chelator, and wherein said radionuclide is Pb-212 or Pb-203. In one embodiment, said radionuclide is Pb-212. In another embodiment, said radionuclide is Pb-203.

In another aspect, the invention relates to such a conjugate or pharmaceutically acceptable salt thereof, wherein said conjugate has the formula: D-Co-Ch-R, wherein D is said ankyrin repeat domain with binding specificity for DLL3, Co is said connector, Ch is said chelator, and R is said radionuclide.

In another aspect, the invention relates to such a conjugate or pharmaceutically acceptable salt thereof, wherein said ankyrin repeat domain with binding specificity for DLL3 binds human DLL3 (hDLL3) with a K_{D} value of or below 100 nM, of or below 30 nM, of or below 10 nM, of or below 3 nM, or of or below 1 nM, of or below 300 pM, of or below 100 pM, of or below 30 pM, or of or below 10 pM. Thus, in one embodiment, said ankyrin repeat domain binds hDLL3 with a K_{D} value of or below 100 nM. In one embodiment, said ankyrin repeat domain binds hDLL3 with a K_{D} value of or below 30 nM. In one embodiment, said ankyrin repeat domain binds hDLL3 with a K_{D} value of or below 10 nM. In one embodiment, said ankyrin repeat domain binds hDLL3 with a K_{D} value of or below 3 nM. In one embodiment, said ankyrin repeat domain binds hDLL3 with a K_{D} value or of or below 1 nM. In one embodiment, said ankyrin repeat domain binds hDLL3 with a K_{D} value of or below 300 pM. In one embodiment, said ankyrin repeat domain binds hDLL3 with a K_{D} value of or below 100 pM. In one embodiment, said ankyrin repeat domain binds hDLL3 with a K_{D} value of or below 30 pM. In one embodiment, said ankyrin repeat domain binds hDLL3 with a K_{D} value of or below 10 pM. Furthermore, in one embodiment, said ankyrin repeat domain binds to the extracellular domain of hDLL3. In one embodiment, said ankyrin repeat domain binds to the N-terminal domain of hDLL3.

In another aspect, the invention relates to such a conjugate or pharmaceutically acceptable salt thereof, wherein said ankyrin repeat domain with binding specificity for DLL3 has a melting temperature (Tm) of at least about 70° C., at least about 75° C., at least about 80° C., at least about 82° C., at least about 85° C., at least about 88° C., or at least about 90° C. Thus, in one embodiment, said ankyrin repeat domain with binding specificity for DLL3 has a melting temperature (Tm) of at least about 70° C. In one embodiment, said ankyrin repeat domain with binding specificity for DLL3 has a melting temperature (Tm) of at least about 75° C. In one embodiment, said ankyrin repeat domain with binding specificity for DLL3 has a melting temperature (Tm) of at least about 80° C. In one embodiment, said ankyrin repeat domain with binding specificity for DLL3 has a melting temperature (Tm) of at least about 82° C. In one embodiment, said ankyrin repeat domain with binding specificity for DLL3 has a melting temperature (Tm) of at least about 85° C.

In one embodiment, said ankyrin repeat domain with binding specificity for DLL3 has a melting temperature (Tm) of at least about 88° C. In one embodiment, said ankyrin repeat domain with binding specificity for DLL3 has a melting temperature (Tm) of at least about 90° C. In one embodiment, said melting temperature (Tm) of said ankyrin repeat domain with binding specificity for DLL3 is determined in PBS. In one embodiment, said melting temperature (Tm) of said ankyrin repeat domain with binding specificity for DLL3 is determined by Circular Dichroism (CD) spectroscopy.

In another aspect, the invention relates to such a conjugate or pharmaceutically acceptable salt thereof, wherein said ankyrin repeat domain with binding specificity for DLL3 comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 1 to 4. Thus, in one embodiment, said ankyrin repeat domain comprises an amino acid sequence that is at least 80% identical to any one of SEQ ID NOs: 1 to 4. In one embodiment, said ankyrin repeat domain comprises an amino acid sequence that is at least 85% identical to any one of SEQ ID NOs: 1 to 4. In one embodiment, said ankyrin repeat domain comprises an amino acid sequence that is at least 88% identical to any one of SEQ ID NOs: 1 to 4. In one embodiment, said ankyrin repeat domain comprises an amino acid sequence that is at least 90% identical to any one of SEQ ID NOs: 1 to 4. In one embodiment, said ankyrin repeat domain comprises an amino acid sequence that is at least 91% identical to any one of SEQ ID NOs: 1 to 4. In one embodiment, said ankyrin repeat domain comprises an amino acid sequence that is at least 92% identical to any one of SEQ ID NOs: 1 to 4. In one embodiment, said ankyrin repeat domain comprises an amino acid sequence that is at least 93% identical to any one of SEQ ID NOs: 1 to 4. In one embodiment, said ankyrin repeat domain comprises an amino acid sequence that is at least 94% identical to any one of SEQ ID NOs: 1 to 4. In one embodiment, said ankyrin repeat domain comprises an amino acid sequence that is at least 95% identical to any one of SEQ ID NOs: 1 to 4. In one embodiment, said ankyrin repeat domain comprises an amino acid sequence that is at least 96% identical to any one of SEQ ID NOs: 1 to 4. In one embodiment, said ankyrin repeat domain comprises an amino acid sequence that is at least 97% identical to any one of SEQ ID NOs: 1 to 4. In one embodiment, said ankyrin repeat domain comprises an amino acid sequence that is at least 98% identical to any one of SEQ ID NOs: 1 to 4. In one embodiment, said ankyrin repeat domain comprises an amino acid sequence that is at least 99% identical to any one of SEQ ID NOs: 1 to 4. In one embodiment, any amino acid sequence differences between said ankyrin repeat domain and said any one of SEQ ID NOs: 1 to 4 represent amino acid substitutions in framework positions. In one embodiment, said ankyrin repeat domain comprises an amino acid sequence that is 100% identical to any one of SEQ ID NOs: 1 to 4.

In another aspect, the invention relates to such a conjugate or pharmaceutically acceptable salt thereof, wherein said ankyrin repeat domain with binding specificity for DLL3 comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 1. Thus, in one embodiment, said ankyrin repeat domain comprises an amino acid sequence that is at least 80% identical to SEQ ID NO: 1. In one embodiment, said ankyrin repeat domain comprises an amino acid sequence that is at least 81% identical to SEQ ID NO: 1. In one embodiment, said ankyrin repeat domain comprises an amino acid sequence that is at least 82% identical to SEQ ID NO: 1. In one embodiment, said ankyrin repeat domain comprises an amino acid sequence that is at least 83% identical to SEQ ID NO: 1. In one embodiment, said ankyrin repeat domain comprises an amino acid sequence that is at least 84% identical to SEQ ID NO: 1. In one embodiment, said ankyrin repeat domain comprises an amino acid sequence that is at least 85% identical to SEQ ID NO: 1. In one embodiment, said ankyrin repeat domain comprises an amino acid sequence that is at least 86% identical to SEQ ID NO: 1. In one embodiment, said ankyrin repeat domain comprises an amino acid sequence that is at least 87% identical to SEQ ID NO: 1. In one embodiment, said ankyrin repeat domain comprises an amino acid sequence that is at least 88% identical to SEQ ID NO: 1. In one embodiment, said ankyrin repeat domain comprises an amino acid sequence that is at least 89% identical to SEQ ID NO: 1. In one embodiment, said ankyrin repeat domain comprises an amino acid sequence that is at least 90% identical to SEQ ID NO: 1. In one embodiment, said ankyrin repeat domain comprises an amino acid sequence that is at least 91% identical to SEQ ID NO: 1. In one embodiment, said ankyrin repeat domain comprises an amino acid sequence that is at least 92% identical to SEQ ID NO: 1. In one embodiment, said ankyrin repeat domain comprises an amino acid sequence that is at least 93% identical SEQ ID NO: 1. In one embodiment, said ankyrin repeat domain comprises an amino acid sequence that is at least 94% identical to SEQ ID NO: 1. In one embodiment, said ankyrin repeat domain comprises an amino acid sequence that is at least 95% identical to SEQ ID NO: 1. In one embodiment, said ankyrin repeat domain comprises an amino acid sequence that is at least 96% identical to SEQ ID NO: 1. In one embodiment, said ankyrin repeat domain comprises an amino acid sequence that is at least 97% identical to SEQ ID NO: 1. In one embodiment, said ankyrin repeat domain comprises an amino acid sequence that is at least 98% identical to SEQ ID NO: 1. In one embodiment, said ankyrin repeat domain comprises an amino acid sequence that is at least 99% identical to SEQ ID NO: 1. In one embodiment, any amino acid sequence differences between said ankyrin repeat domain and SEQ ID NO: 1 represent amino acid substitutions in framework positions. In one embodiment, said ankyrin repeat domain comprises an amino acid sequence that is 100% identical to SEQ ID NO: 1.

In another aspect, the invention relates to such a conjugate or pharmaceutically acceptable salt thereof, wherein said ankyrin repeat domain with binding specificity for DLL3 comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 2. Thus, in one embodiment, said ankyrin repeat domain comprises an amino acid sequence that is at least 80% identical to SEQ ID NO: 2. In one embodiment, said ankyrin repeat domain comprises an amino acid sequence that is at least 85% identical to SEQ ID NO: 2. In one embodiment, said ankyrin repeat domain comprises an amino acid sequence that is at least 88% identical to SEQ ID NO: 2. In one embodiment, said ankyrin repeat domain comprises an amino acid sequence that is at least 90% identical to SEQ ID NO: 2. In one embodiment, said ankyrin repeat domain comprises an amino acid sequence that is at least 91% identical to SEQ ID NO: 2. In one embodiment, said ankyrin repeat domain comprises an amino acid sequence that is at least 92% identical to SEQ ID NO: 2. In one embodiment, said ankyrin repeat domain comprises an amino acid sequence that is at least 93% identical SEQ ID NO: 2. In one embodiment, said ankyrin repeat domain comprises an amino acid sequence that is at least 94% identical to SEQ ID NO: 2. In one embodiment, said ankyrin repeat domain comprises an amino acid sequence that is at least 95% identical to SEQ ID NO: 2. In one embodiment, said ankyrin repeat domain comprises an amino acid sequence that is at least 96% identical to SEQ ID NO: 2. In one embodiment, said ankyrin repeat domain comprises an amino acid sequence that is at least 97% identical to SEQ ID NO: 2. In one embodiment, said ankyrin repeat domain comprises an amino acid sequence that is at least 98% identical to SEQ ID NO: 2. In one embodiment, said ankyrin repeat domain comprises an amino acid sequence that is at least 99% identical to SEQ ID NO: 2. In one embodiment, any amino acid sequence differences between said ankyrin repeat domain and SEQ ID NO: 2 represent amino acid substitutions in framework positions. In one embodiment, said ankyrin repeat domain comprises an amino acid sequence that is 100% identical to SEQ ID NO: 2.

In another aspect, the invention relates to such a conjugate or pharmaceutically acceptable salt thereof, wherein said ankyrin repeat domain with binding specificity for DLL3 comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 3. Thus, in one embodiment, said ankyrin repeat domain comprises an amino acid sequence that is at least 80% identical to SEQ ID NO: 3. In one embodiment, said ankyrin repeat domain comprises an amino acid sequence that is at least 85% identical to SEQ ID NO: 3. In one embodiment, said ankyrin repeat domain comprises an amino acid sequence that is at least 88% identical to SEQ ID NO: 3. In one embodiment, said ankyrin repeat domain comprises an amino acid sequence that is at least 90% identical to SEQ ID NO: 3. In one embodiment, said ankyrin repeat domain comprises an amino acid sequence that is at least 91% identical to SEQ ID NO: 3. In one embodiment, said ankyrin repeat domain comprises an amino acid sequence that is at least 92% identical to SEQ ID NO: 3. In one embodiment, said ankyrin repeat domain comprises an amino acid sequence that is at least 93% identical SEQ ID NO: 3. In one embodiment, said ankyrin repeat domain comprises an amino acid sequence that is at least 94% identical to SEQ ID NO: 3. In one embodiment, said ankyrin repeat domain comprises an amino acid sequence that is at least 95% identical to SEQ ID NO: 3. In one embodiment, said ankyrin repeat domain comprises an amino acid sequence that is at least 96% identical to SEQ ID NO: 3. In one embodiment, said ankyrin repeat domain comprises an amino acid sequence that is at least 97% identical to SEQ ID NO: 3. In one embodiment, said ankyrin repeat domain comprises an amino acid sequence that is at least 98% identical to SEQ ID NO: 3. In one embodiment, said ankyrin repeat domain comprises an amino acid sequence that is at least 99% identical to SEQ ID NO: 3. In one embodiment, any amino acid sequence differences between said ankyrin repeat domain and SEQ ID NO: 3 represent amino acid substitutions in framework positions. In one embodiment, said ankyrin repeat domain comprises an amino acid sequence that is 100% identical to SEQ ID NO: 3.

In another aspect, the invention relates to such a conjugate or pharmaceutically acceptable salt thereof, wherein said ankyrin repeat domain with binding specificity for DLL3 comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 4. Thus, in one embodiment, said ankyrin repeat domain comprises an amino acid sequence that is at least 80% identical to SEQ ID NO: 4. In one embodiment, said ankyrin repeat domain comprises an amino acid sequence that is at least 85% identical to SEQ ID NO: 4. In one embodiment, said ankyrin repeat domain comprises an amino acid sequence that is at least 88% identical to SEQ ID NO: 4. In one embodiment, said ankyrin repeat domain comprises an amino acid sequence that is at least 90% identical to SEQ ID NO: 4. In one embodiment, said ankyrin repeat domain comprises an amino acid sequence that is at least 91% identical to SEQ ID NO: 4. In one embodiment, said ankyrin repeat domain comprises an amino acid sequence that is at least 92% identical to SEQ ID NO: 4. In one embodiment, said ankyrin repeat domain comprises an amino acid sequence that is at least 93% identical SEQ ID NO: 4. In one embodiment, said ankyrin repeat domain comprises an amino acid sequence that is at least 94% identical to SEQ ID NO: 4. In one embodiment, said ankyrin repeat domain comprises an amino acid sequence that is at least 95% identical to SEQ ID NO: 4. In one embodiment, said ankyrin repeat domain comprises an amino acid sequence that is at least 96% identical to SEQ ID NO: 4. In one embodiment, said ankyrin repeat domain comprises an amino acid sequence that is at least 97% identical to SEQ ID NO: 4. In one embodiment, said ankyrin repeat domain comprises an amino acid sequence that is at least 98% identical to SEQ ID NO: 4. In one embodiment, said ankyrin repeat domain comprises an amino acid sequence that is at least 99% identical to SEQ ID NO: 4. In one embodiment, any amino acid sequence differences between said ankyrin repeat domain and SEQ ID NO: 4 represent amino acid substitutions in framework positions. In one embodiment, said ankyrin repeat domain comprises an amino acid sequence that is 100% identical to SEQ ID NO: 4.

In another aspect, the invention relates to such a conjugate or pharmaceutically acceptable salt thereof, wherein said ankyrin repeat domain with binding specificity for DLL3 comprises an amino acid sequence selected from the group consisting of (1) SEQ ID NOs: 1 to 4 and (2) sequences in which up to 25, or up to 24, or up to 23, or up to 22, or up to 21, or up to 20, or up to 19, or up to 18, or up to 17, or up to 16, or up to 15, or up to 14, or up to 13, or up to 12, or up to 11, or up to 10, or up to 9, or up to 8, or up to 7, or up to 6, or up to 5, or up to 4, or up to 3, or up to 2, or up to 1 amino acids in any of SEQ ID NOs: 1 to 4 are substituted by other amino acids. Thus, in one embodiment, said ankyrin repeat domain comprises an amino acid sequence selected from the group consisting of (1) SEQ ID NOs: 1 to 4 and (2) sequences in which up to 25 amino acids in any of SEQ ID NOs: 1 to 4 are substituted by other amino acids. In one embodiment, said ankyrin repeat domain comprises an amino acid sequence selected from the group consisting of (1) SEQ ID NOs: 1 to 4 and (2) sequences in which up to 20 amino acids in any of SEQ ID NOs: 1 to 4 are substituted by other amino acids. In one embodiment, said ankyrin repeat domain comprises an amino acid sequence selected from the group consisting of (1) SEQ ID NOs: 1 to 4 and (2) sequences in which up to 15 amino acids in any of SEQ ID NOs: 1 to 4 are substituted by other amino acids. In one embodiment, said ankyrin repeat domain comprises an amino acid sequence selected from the group consisting of (1) SEQ ID NOs: 1 to 4 and (2) sequences in which up to 10 amino acids in any of SEQ ID NOs: 1 to 4 are substituted by other amino acids. In one embodiment, said ankyrin repeat domain comprises an amino acid sequence selected from the group consisting of (1) SEQ ID NOs: 1 to 4 and (2) sequences in which up to 5 amino acids in any of SEQ ID NOs: 1 to 4 are substituted by other amino acids. In one embodiment, said ankyrin repeat domain comprises an amino acid sequence selected from the group consisting of (1) SEQ ID NOs: 1 to 4 and (2) sequences in which up to 4 amino acids in any of SEQ ID NOs: 1 to 4 are substituted by other amino acids. In one embodiment, said ankyrin repeat domain comprises an amino acid sequence selected from the group consisting of (1) SEQ ID NOs: 1 to 4 and (2) sequences in which up to 3 amino acids in any of SEQ ID NOs: 1 to 4 are substituted by other amino acids. In one embodiment, said ankyrin repeat domain comprises an amino acid sequence selected from the group consisting of (1) SEQ ID NOs: 1 to 4 and (2) sequences in which up to 2 amino acids in any of SEQ ID NOs: 1 to 4 are substituted by other amino acids. In one embodiment, said ankyrin repeat domain comprises an amino acid sequence selected from the group consisting of (1) SEQ ID NOs: 1 to 4 and (2) sequences in which up to 1 amino acid in any of SEQ ID NOs: 1 to 4 is substituted by another amino acid. In one embodiment, all of said 25, 24, 23, 22, 21, 20, 19, 18, 17, 16, 15, 14, 13, 12, 11, 10, 9, 8, 7, 6, 5, 4, 3, 2, or 1 amino acid substitutions occur in framework positions. In one embodiment, said ankyrin repeat domain comprises an amino acid sequence selected from the group consisting of SEQ ID NOs: 1 to 4.

In another aspect, the invention relates to such a conjugate or pharmaceutically acceptable salt thereof, wherein said ankyrin repeat domain with binding specificity for DLL3 comprises an amino acid sequence selected from the group consisting of (1) SEQ ID NO: 1 and (2) sequences in which up to 25, or up to 24, or up to 23, or up to 22, or up to 21, or up to 20, or up to 19, or up to 18, or up to 17, or up to 16, or up to 15, or up to 14, or up to 13, or up to 12, or up to 11, or up to 10, or up to 9, or up to 8, or up to 7, or up to 6, or up to 5, or up to 4, or up to 3, or up to 2, or up to 1 amino acids of SEQ ID NO: 1 are substituted by other amino acids. Thus, in one embodiment, said ankyrin repeat domain comprises an amino acid sequence selected from the group consisting of (1) SEQ ID NO: 1 and (2) sequences in which up to 25 amino acids of SEQ ID NO: 1 are substituted by other amino acids. In one embodiment, said ankyrin repeat domain comprises an amino acid sequence selected from the group consisting of (1) SEQ ID NO: 1 and (2) sequences in which up to 20 amino acids of SEQ ID NO: 1 are substituted by other amino acids. In one embodiment, said ankyrin repeat domain comprises an amino acid sequence selected from the group consisting of (1) SEQ ID NO: 1 and (2) sequences in which up to 19 amino acids of SEQ ID NO: 1 are substituted by other amino acids. In one embodiment, said ankyrin repeat domain comprises an amino acid sequence selected from the group consisting of (1) SEQ ID NO: 1 and (2) sequences in which up to 18 amino acids of SEQ ID NO: 1 are substituted by other amino acids. In one embodiment, said ankyrin repeat domain comprises an amino acid sequence selected from the group consisting of (1) SEQ ID NO: 1 and (2) sequences in which up to 17 amino acids of SEQ ID NO: 1 are substituted by other amino acids. In one embodiment, said ankyrin repeat domain comprises an amino acid sequence selected from the group consisting of (1) SEQ ID NO: 1 and (2) sequences in which up to 16 amino acids of SEQ ID NO: 1 are substituted by other amino acids. In one embodiment, said ankyrin repeat domain comprises an amino acid sequence selected from the group consisting of (1) SEQ ID NO: 1 and (2) sequences in which up to 15 amino acids of SEQ ID NO: 1 are substituted by other amino acids. In one embodiment, said ankyrin repeat domain comprises an amino acid sequence selected from the group consisting of (1) SEQ ID NO: 1 and (2) sequences in which up to 14 amino acids of SEQ ID NO: 1 are substituted by other amino acids. In one embodiment, said ankyrin repeat domain comprises an amino acid sequence selected from the group consisting of (1) SEQ ID NO: 1 and (2) sequences in which up to 13 amino acids of SEQ ID NO: 1 are substituted by other amino acids. In one embodiment, said ankyrin repeat domain comprises an amino acid sequence selected from the group consisting of (1) SEQ ID NO: 1 and (2) sequences in which up to 12 amino acids of SEQ ID NO: 1 are substituted by other amino acids. In one embodiment, said ankyrin repeat domain comprises an amino acid sequence selected from the group consisting of (1) SEQ ID NO: 1 and (2) sequences in which up to 11 amino acids of SEQ ID NO: 1 are substituted by other amino acids. In one embodiment, said ankyrin repeat domain comprises an amino acid sequence selected from the group consisting of (1) SEQ ID NO: 1 and (2) sequences in which up to 10 amino acids of SEQ ID NO: 1 are substituted by other amino acids. In one embodiment, said ankyrin repeat domain comprises an amino acid sequence selected from the group consisting of (1) SEQ ID NO: 1 and (2) sequences in which up to 9 amino acids of SEQ ID NO: 1 are substituted by other amino acids. In one embodiment, said ankyrin repeat domain comprises an amino acid sequence selected from the group consisting of (1) SEQ ID NO: 1 and (2) sequences in which up to 8 amino acids of SEQ ID NO: 1 are substituted by other amino acids. In one embodiment, said ankyrin repeat domain comprises an amino acid sequence selected from the group consisting of (1) SEQ ID NO: 1 and (2) sequences in which up to 7 amino acids of SEQ ID NO: 1 are substituted by other amino acids. In one embodiment, said ankyrin repeat domain comprises an amino acid sequence selected from the group consisting of (1) SEQ ID NO: 1 and (2) sequences in which up to 6 amino acids of SEQ ID NO: 1 are substituted by other amino acids. In one embodiment, said ankyrin repeat domain comprises an amino acid sequence selected from the group consisting of (1) SEQ ID NO: 1 and (2) sequences in which up to 5 amino acids of SEQ ID NO: 1 are substituted by other amino acids. In one embodiment, said ankyrin repeat domain comprises an amino acid sequence selected from the group consisting of (1) SEQ ID NO: 1 and (2) sequences in which up to 4 amino acids of SEQ ID NO: 1 are substituted by other amino acids. In one embodiment, said ankyrin repeat domain comprises an amino acid sequence selected from the group consisting of (1) SEQ ID NO: 1 and (2) sequences in which up to 3 amino acids of SEQ ID NO: 1 are substituted by other amino acids. In one embodiment, said ankyrin repeat domain comprises an amino acid sequence selected from the group consisting of (1) SEQ ID NO: 1 and (2) sequences in which up to 2 amino acids of SEQ ID NO: 1 are substituted by other amino acids. In one embodiment, said ankyrin repeat domain comprises an amino acid sequence selected from the group consisting of (1) SEQ ID NO: 1 and (2) sequences in which up to 1 amino acid of SEQ ID NO: 1 is substituted by another amino acid. In one embodiment, all of said 25, 24, 23, 22, 21, 20, 19, 18, 17, 16, 15, 14, 13, 12, 11, 10, 9, 8, 7, 6, 5, 4, 3, 2, or 1 amino acid substitutions occur in framework positions. In one embodiment, said ankyrin repeat domain comprises the amino acid sequence of SEQ ID NO: 1.

In another aspect, the invention relates to such a conjugate or pharmaceutically acceptable salt thereof, wherein said ankyrin repeat domain with binding specificity for DLL3 comprises an amino acid sequence selected from the group consisting of (1) SEQ ID NO: 2 and (2) sequences in which up to 25, or up to 24, or up to 23, or up to 22, or up to 21, or up to 20, or up to 19, or up to 18, or up to 17, or up to 16, or up to 15, or up to 14, or up to 13, or up to 12, or up to 11, or up to 10, or up to 9, or up to 8, or up to 7, or up to 6, or up to 5, or up to 4, or up to 3, or up to 2, or up to 1 amino acids of SEQ ID NO: 2 are substituted by other amino acids. Thus, in one embodiment, said ankyrin repeat domain comprises an amino acid sequence selected from the group consisting of (1) SEQ ID NO: 2 and (2) sequences in which up to 25 amino acids of SEQ ID NO: 2 are substituted by other amino acids. In one embodiment, said ankyrin repeat domain comprises an amino acid sequence selected from the group consisting of (1) SEQ ID NO: 2 and (2) sequences in which up to 20 amino acids of SEQ ID NO: 2 are substituted by other amino acids. In one embodiment, said ankyrin repeat domain comprises an amino acid sequence selected from the group consisting of (1) SEQ ID NO: 2 and (2) sequences in which up to 15 amino acids of SEQ ID NO: 2 are substituted by other amino acids. In one embodiment, said ankyrin repeat domain comprises an amino acid sequence selected from the group consisting of (1) SEQ ID NO: 2 and (2) sequences in which up to 10 amino acids of SEQ ID NO: 2 are substituted by other amino acids. In one embodiment, said ankyrin repeat domain comprises an amino acid sequence selected from the group consisting of (1) SEQ ID NO: 2 and (2) sequences in which up to 5 amino acids of SEQ ID NO: 2 are substituted by other amino acids. In one embodiment, said ankyrin repeat domain comprises an amino acid sequence selected from the group consisting of (1) SEQ ID NO: 2 and (2) sequences in which up to 4 amino acids of SEQ ID NO: 2 are substituted by other amino acids. In one embodiment, said ankyrin repeat domain comprises an amino acid sequence selected from the group consisting of (1) SEQ ID NO: 2 and (2) sequences in which up to 3 amino acids of SEQ ID NO: 2 are substituted by other amino acids. In one embodiment, said ankyrin repeat domain comprises an amino acid sequence selected from the group consisting of (1) SEQ ID NO: 2 and (2) sequences in which up to 2 amino acids of SEQ ID NO: 2 are substituted by other amino acids. In one embodiment, said ankyrin repeat domain comprises an amino acid sequence selected from the group consisting of (1) SEQ ID NO: 2 and (2) sequences in which up to 1 amino acid of SEQ ID NO: 2 is substituted by another amino acid. In one embodiment, all of said 25, 24, 23, 22, 21, 20, 19, 18, 17, 16, 15, 14, 13, 12, 11, 10, 9, 8, 7, 6, 5, 4, 3, 2, or 1 amino acid substitutions occur in framework positions. In one embodiment, said ankyrin repeat domain comprises the amino acid sequence of SEQ ID NO: 2.

In another aspect, the invention relates to such a conjugate or pharmaceutically acceptable salt thereof, wherein said ankyrin repeat domain with binding specificity for DLL3 comprises an amino acid sequence selected from the group consisting of (1) SEQ ID NO: 3 and (2) sequences in which up to 25, or up to 24, or up to 23, or up to 22, or up to 21, or up to 20, or up to 19, or up to 18, or up to 17, or up to 16, or up to 15, or up to 14, or up to 13, or up to 12, or up to 11, or up to 10, or up to 9, or up to 8, or up to 7, or up to 6, or up to 5, or up to 4, or up to 3, or up to 2, or up to 1 amino acids of SEQ ID NO: 3 are substituted by other amino acids. Thus, in one embodiment, said ankyrin repeat domain comprises an amino acid sequence selected from the group consisting of (1) SEQ ID NO: 3 and (2) sequences in which up to 25 amino acids of SEQ ID NO: 3 are substituted by other amino acids. In one embodiment, said ankyrin repeat domain comprises an amino acid sequence selected from the group consisting of (1) SEQ ID NO: 3 and (2) sequences in which up to 20 amino acids of SEQ ID NO: 3 are substituted by other amino acids. In one embodiment, said ankyrin repeat domain comprises an amino acid sequence selected from the group consisting of (1) SEQ ID NO: 3 and (2) sequences in which up to 15 amino acids of SEQ ID NO: 3 are substituted by other amino acids. In one embodiment, said ankyrin repeat domain comprises an amino acid sequence selected from the group consisting of (1) SEQ ID NO: 3 and (2) sequences in which up to 10 amino acids of SEQ ID NO: 3 are substituted by other amino acids. In one embodiment, said ankyrin repeat domain comprises an amino acid sequence selected from the group consisting of (1) SEQ ID NO: 3 and (2) sequences in which up to 5 amino acids of SEQ ID NO: 3 are substituted by other amino acids. In one embodiment, said ankyrin repeat domain comprises an amino acid sequence selected from the group consisting of (1) SEQ ID NO: 3 and (2) sequences in which up to 4 amino acids of SEQ ID NO: 3 are substituted by other amino acids. In one embodiment, said ankyrin repeat domain comprises an amino acid sequence selected from the group consisting of (1) SEQ ID NO: 3 and (2) sequences in which up to 3 amino acids of SEQ ID NO: 3 are substituted by other amino acids. In one embodiment, said ankyrin repeat domain comprises an amino acid sequence selected from the group consisting of (1) SEQ ID NO: 3 and (2) sequences in which up to 2 amino acids of SEQ ID NO: 3 are substituted by other amino acids. In one embodiment, said ankyrin repeat domain comprises an amino acid sequence selected from the group consisting of (1) SEQ ID NO: 3 and (2) sequences in which up to 1 amino acid of SEQ ID NO: 3 is substituted by another amino acid. In one embodiment, all of said 25, 24, 23, 22, 21, 20, 19, 18, 17, 16, 15, 14, 13, 12, 11, 10, 9, 8, 7, 6, 5, 4, 3, 2, or 1 amino acid substitutions occur in framework positions. In one embodiment, said ankyrin repeat domain comprises the amino acid sequence of SEQ ID NO: 3.

In another aspect, the invention relates to such a conjugate or pharmaceutically acceptable salt thereof, wherein said ankyrin repeat domain with binding specificity for DLL3 comprises an amino acid sequence selected from the group consisting of (1) SEQ ID NO: 4 and (2) sequences in which up to 25, or up to 24, or up to 23, or up to 22, or up to 21, or up to 20, or up to 19, or up to 18, or up to 17, or up to 16, or up to 15, or up to 14, or up to 13, or up to 12, or up to 11, or up to 10, or up to 9, or up to 8, or up to 7, or up to 6, or up to 5, or up to 4, or up to 3, or up to 2, or up to 1 amino acids of SEQ ID NO: 4 are substituted by other amino acids. Thus, in one embodiment, said ankyrin repeat domain comprises an amino acid sequence selected from the group consisting of (1) SEQ ID NO: 4 and (2) sequences in which up to 25 amino acids of SEQ ID NO: 4 are substituted by other amino acids. In one embodiment, said ankyrin repeat domain comprises an amino acid sequence selected from the group consisting of (1) SEQ ID NO: 4 and (2) sequences in which up to 20 amino acids of SEQ ID NO: 4 are substituted by other amino acids. In one embodiment, said ankyrin repeat domain comprises an amino acid sequence selected from the group consisting of (1) SEQ ID NO: 4 and (2) sequences in which up to 15 amino acids of SEQ ID NO: 4 are substituted by other amino acids. In one embodiment, said ankyrin repeat domain comprises an amino acid sequence selected from the group consisting of (1) SEQ ID NO: 4 and (2) sequences in which up to 10 amino acids of SEQ ID NO: 4 are substituted by other amino acids. In one embodiment, said ankyrin repeat domain comprises an amino acid sequence selected from the group consisting of (1) SEQ ID NO: 4 and (2) sequences in which up to 5 amino acids of SEQ ID NO: 4 are substituted by other amino acids. In one embodiment, said ankyrin repeat domain comprises an amino acid sequence selected from the group consisting of (1) SEQ ID NO: 4 and (2) sequences in which up to 4 amino acids of SEQ ID NO: 4 are substituted by other amino acids. In one embodiment, said ankyrin repeat domain comprises an amino acid sequence selected from the group consisting of (1) SEQ ID NO: 4 and (2) sequences in which up to 3 amino acids of SEQ ID NO: 4 are substituted by other amino acids. In one embodiment, said ankyrin repeat domain comprises an amino acid sequence selected from the group consisting of (1) SEQ ID NO: 4 and (2) sequences in which up to 2 amino acids of SEQ ID NO: 4 are substituted by other amino acids. In one embodiment, said ankyrin repeat domain comprises an amino acid sequence selected from the group consisting of (1) SEQ ID NO: 4 and (2) sequences in which up to 1 amino acid of SEQ ID NO: 4 is substituted by another amino acid. In one embodiment, all of said 25, 24, 23, 22, 21, 20, 19, 18, 17, 16, 15, 14, 13, 12, 11, 10, 9, 8, 7, 6, 5, 4, 3, 2, or 1 amino acid substitutions occur in framework positions. In one embodiment, said ankyrin repeat domain comprises the amino acid sequence of SEQ ID NO: 4.

In any of the conjugates, or pharmaceutically acceptable salts thereof, of the invention described herein, said ankyrin repeat domain with binding specificity for DLL3 may optionally further comprise a “G,” an “S,” or a “GS” sequence at its N-terminus. Accordingly, in some embodiments, said ankyrin repeat domain with binding specificity for DLL3 (i) comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 1 to 4 and (ii) further comprises at its N-terminus, a G, an S, or a GS. In some embodiments, said ankyrin repeat domain with binding specificity for DLL3 (i) comprises an amino acid sequence selected from the group consisting of (1) SEQ ID NOs: 1 to 4 and (2) sequences in which up to 25, or up to 24, or up to 23, or up to 22, or up to 21, or up to 20, or up to 19, or up to 18, or up to 17, or up to 16, or up to 15, or up to 14, or up to 13, or up to 12, or up to 11, or up to 10, or up to 9, or up to 8, or up to 7, or up to 6, or up to 5, or up to 4, or up to 3, or up to 2, or up to 1 amino acids in any of SEQ ID NOs: 1 to 4 are substituted by other amino acids, and (ii) further comprises at its N-terminus, a G, an S, or a GS. Thus, in an exemplary embodiment, said ankyrin repeat domain with binding specificity for DLL3 (i) comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 1 and (ii) further comprises a GS at its N-terminus. In another exemplary embodiment, said ankyrin repeat domain with binding specificity for DLL3 (i) comprises an amino acid sequence selected from the group consisting of (1) SEQ ID NO: 1 and (2) sequences in which up to 25, or up to 24, or up to 23, or up to 22, or up to 21, or up to 20, or up to 19, or up to 18, or up to 17, or up to 16, or up to 15, or up to 14, or up to 13, or up to 12, or up to 11, or up to 10, or up to 9, or up to 8, or up to 7, or up to 6, or up to 5, or up to 4, or up to 3, or up to 2, or up to 1 amino acids in SEQ ID NO: 1 are substituted by other amino acids, and (ii) further comprises a GS at its N-terminus.

In another aspect, the invention relates to such a conjugate or pharmaceutically acceptable salt thereof, wherein said chelator comprises a 1,4,7,10-tetraazacyclododecane ring (PubChem CID 64963), or a derivative thereof. In one embodiment, said 1,4,7,10-tetraazacyclododecane ring comprises one or more side chains. In one embodiment, said one or more side chains are connected to one or more of the nitrogen atoms of said 1,4,7,10-tetraazacyclododecane ring. In one embodiment, said 1,4,7,10-tetraazacyclododecane ring comprises one, two, three or four side chains, wherein each of said side chains is connected to a nitrogen atom of said 1,4,7,10-tetraazacyclododecane ring. In one embodiment, at least one of said one or more side chains comprises a carboxyl group (—COOH) or an amide group (—CONH₂). In one embodiment, at least one of said one or more side chains comprises a —CH₂—COOH group or a —CH₂—CONH₂ group. In one embodiment, said 1,4,7,10-tetraazacyclododecane ring comprises four side chains, wherein each of said side chains is connected to a nitrogen atom of said 14,7,10-tetraazacyclododecane ring, and wherein each of said side chains comprises a carboxyl group (—COOH) or an amide group (—CONH₂). In one embodiment, said 1,4,7,10-tetraazacyclododecane ring comprises four side chains, wherein each of said side chains is connected to a nitrogen atom of said 1,4,7,10-tetraazacyclododecane ring, and wherein each of said side chains comprises a —CH₂—COOH group or a —CH₂—CONH₂ group. In one embodiment, said chelator is DOTA (1,4,7,10-tetraazacyclododecane-1,4,7,10-tetraacetic acid) or TCMC (1,4,7,10-Tetraazacyclododecane-1,4,7,10-tetraacetamide), or a derivative thereof. In one embodiment, said chelator is DOTA (1,4,7,10-tetraazacyclododecane-1,4,7,10-tetraacetic acid), or a derivative thereof. In one embodiment, said chelator is TCMC (1,4,7,10-Tetraazacyclododecane-1,4,7,10-tetraacetamide), or a derivative thereof. TCMC is also called DOTAM or DOTA-amide. Derivatives of TCMC include, for example, monoacid forms of TCMC. Thus, in one exemplary embodiment, said chelator comprises a 1,4,7,10-tetraazacyclododecane ring, wherein said 1,4,7,10-tetraazacyclododecane ring comprises four side chains, wherein each of said side chains is connected to a nitrogen atom of said 1,4,7,10-tetraazacyclododecane ring, and wherein one of said side chains comprises a —CH₂—COOH group and at least one of said side chains comprises a —CH₂—CONH₂ group.

In another aspect, the invention relates to such a conjugate or pharmaceutically acceptable salt thereof, wherein said chelator comprises a 1,4,7,10-tetraazacyclododecane ring, wherein said 1,4,7,10-tetraazacyclododecane ring comprises one or more side chains, wherein said one or more side chains are connected to one or more of the nitrogen atoms of said 1,4,7,10-tetraazacyclododecane ring, and wherein said chelator is covalently connected to said connector via one of said side chains. In one embodiment, said chelator has a structure of Formula (I):

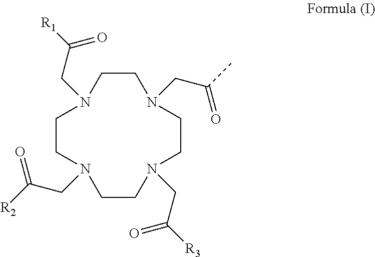

wherein R1, R2 and R3 are independently NH₂ or OH, and wherein the dotted line represents the covalent connection to said ankyrin repeat domain with binding specificity for DLL3 or said connector. In one embodiment, said chelator has a structure of Formula (II):

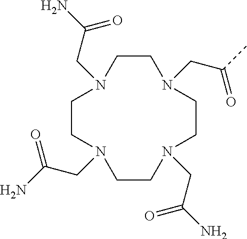

wherein the dotted line represents the covalent connection to said ankyrin repeat domain with binding specificity for DLL3 or said connector.

In another aspect, the invention relates to such a conjugate or pharmaceutically acceptable salt thereof, wherein said chelator comprises a 1,4,7,10-tetraazacyclododecane ring, and wherein said chelator is covalently connected to said connector via one of the carbon atoms of said 1,4,7,10-tetraazacyclododecane ring. In one embodiment, said chelator has a structure of Formula (III):

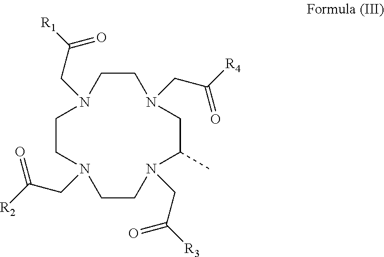

wherein R1, R2, R3 and R4 are independently NH₂ or OH, and wherein the dotted line represents the covalent connection to said ankyrin repeat domain with binding specificity for DLL3 or said connector. In one embodiment, said chelator has a structure of Formula (IV):

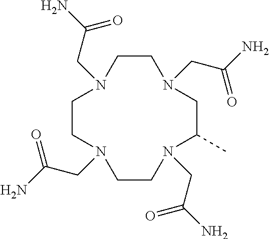 [SMILES: C[C@H]1CN(CC(N)=O)CCN(CC(N)=O)CCN(CC(N)=O)CCN1CC(N)=O]

wherein the dotted line represents the covalent connection to said ankyrin repeat domain with binding specificity for DLL3 or said connector.

In another aspect, the invention relates to such a conjugate or pharmaceutically acceptable salt thereof, wherein said conjugate further comprises a tag, wherein said tag comprises a Cysteine. In one embodiment, said tag is a peptide tag. In one embodiment, said tag is on one side covalently connected to said ankyrin repeat domain with binding specificity for DLL3 and is on another side covalently connected to said connector. In one embodiment, said tag is located at the C-terminal side of said ankyrin repeat domain with binding specificity for DLL3. In one embodiment, said tag is covalently connected by a peptide bond to the C-terminal end of said ankyrin repeat domain with binding specificity for DLL3. In one embodiment, said conjugate has the formula: D-T-Co-Ch-R, wherein D is said ankyrin repeat domain with binding specificity for DLL3, T is said tag, Co is said connector, Ch is said chelator, and R is said radionuclide. In one embodiment, said tag comprises the amino acid sequence of SEQ ID NO: 10 or a variant thereof.

In another aspect, the invention relates to such a conjugate or pharmaceutically acceptable salt thereof, wherein said connector is covalently connected to said tag via a thioether bond. In one embodiment, said connector comprises a maleimide or a derivative thereof. In one embodiment, said thioether bond covalently connecting said tag and said connector is formed between said Cysteine comprised in said tag and said maleimide comprised in said connector. In one embodiment, said connector has a structure of Formula (V):

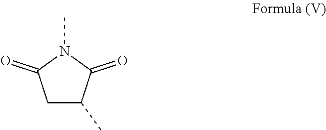

wherein the dotted line originating from N represents the covalent connection to said chelator, and wherein the dotted line originating from a carbon atom represents the covalent connection to said tag.

In another aspect, the invention relates to such a conjugate or pharmaceutically acceptable salt thereof, wherein said conjugate further comprises a half-life extending moiety. In one embodiment, said conjugate has the formula: H-D-Ch-R, wherein H is said half-life extending moiety, D is said ankyrin repeat domain with binding specificity for DLL3, Ch is said chelator, and R is said radionuclide. In one embodiment, said conjugate has the formula: D-H-Ch-R, wherein H is said half-life extending moiety, D is said ankyrin repeat domain with binding specificity for DLL3, Ch is said chelator, and R is said radionuclide. In one embodiment, said conjugate has the formula: H-D-Co-Ch-R, wherein H is said half-life extending moiety, D is said ankyrin repeat domain with binding specificity for DLL3, Co is said connector, Ch is said chelator, and R is said radionuclide. In one embodiment, said conjugate has the formula: H-D-T-Co-Ch-R, wherein H is said half-life extending moiety, D is said ankyrin repeat domain with binding specificity for DLL3, T is said tag, Co is said connector, Ch is said chelator, and R is said radionuclide. In one embodiment, said conjugate has the formula: D-H-Co-Ch-R, wherein H is said half-life extending moiety, D is said ankyrin repeat domain with binding specificity for DLL3, Co is said connector, Ch is said chelator, and R is said radionuclide. In one embodiment, said conjugate has the formula: D-H-T-Co-Ch-R, wherein H is said half-life extending moiety, D is said ankyrin repeat domain with binding specificity for DLL3, T is said tag, Co is said connector, Ch is said chelator, and R is said radionuclide. In one embodiment, said half-life extending moiety comprises an immunoglobulin domain. In one embodiment, the immunoglobulin domain comprises an Fc domain, or a variant thereof. In one embodiment, the Fc domain is derived from any one of the known heavy chain isotypes: IgG (γ), IgM (μ), IgD (δ), IgE (ε), or IgA (α). In another embodiment, the Fc domain is derived from any one of the known heavy chain isotypes or subtypes: IgG₁ (γ1), IgG₂ (γ2), IgG₃ (γ3), IgG₄ (γ4), IgA₁ (α1), or IgA₂ (α2). In one embodiment, the Fc domain is the Fc domain of human IgG₁, or a variant thereof.

In another aspect, the invention relates to such a conjugate or pharmaceutically acceptable salt thereof, wherein said conjugate further comprises a half-life extending moiety, and wherein said half-life extending moiety has binding specificity for human serum albumin. In one embodiment, said half-life extending moiety is an ankyrin repeat domain with binding specificity for human serum albumin. In one embodiment, said ankyrin repeat domain with binding specificity for human serum albumin binds human serum albumin with a K_{D} value of or below 500 nM, of or below 250 nM, or of or below 100 nM. In one embodiment, said ankyrin repeat domain with binding specificity for human serum albumin binds human serum albumin with a K_{D} value of or below 500 nM. In one embodiment, said ankyrin repeat domain with binding specificity for human serum albumin binds human serum albumin with a K_{D} value of or below 250 nM. In one embodiment, said ankyrin repeat domain with binding specificity for human serum albumin binds human serum albumin with a K_{D} value of or below 100 nM.

In another aspect, the invention relates to such a conjugate or pharmaceutically acceptable salt thereof, wherein said ankyrin repeat domain with binding specificity for human serum albumin comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 5 to 7. Thus, in one embodiment, said ankyrin repeat domain with binding specificity for human serum albumin comprises an amino acid sequence that is at least 80% identical to any one of SEQ ID NOs: 5 to 7. In one embodiment, said ankyrin repeat domain with binding specificity for human serum albumin comprises an amino acid sequence that is at least 85% identical to any one of SEQ ID NOs: 5 to 7. In one embodiment, said ankyrin repeat domain with binding specificity for human serum albumin comprises an amino acid sequence that is at least 88% identical to any one of SEQ ID NOs: 5 to 7. In one embodiment, said ankyrin repeat domain with binding specificity for human serum albumin comprises an amino acid sequence that is at least 90% identical to any one of SEQ ID NOs: 5 to 7. In one embodiment, said ankyrin repeat domain with binding specificity for human serum albumin comprises an amino acid sequence that is at least 91% identical to any one of SEQ ID NOs: 5 to 7. In one embodiment, said ankyrin repeat domain with binding specificity for human serum albumin comprises an amino acid sequence that is at least 92% identical to any one of SEQ ID NOs: 5 to 7. In one embodiment, said ankyrin repeat domain with binding specificity for human serum albumin comprises an amino acid sequence that is at least 93% identical to any one of SEQ ID NOs: 5 to 7. In one embodiment, said ankyrin repeat domain with binding specificity for human serum albumin comprises an amino acid sequence that is at least 94% identical to any one of SEQ ID NOs: 5 to 7. In one embodiment, said ankyrin repeat domain with binding specificity for human serum albumin comprises an amino acid sequence that is at least 95% identical to any one of SEQ ID NOs: 5 to 7. In one embodiment, said ankyrin repeat domain with binding specificity for human serum albumin comprises an amino acid sequence that is at least 96% identical to any one of SEQ ID NOs: 5 to 7. In one embodiment, said ankyrin repeat domain with binding specificity for human serum albumin comprises an amino acid sequence that is at least 97% identical to any one of SEQ ID NOs: 5 to 7. In one embodiment, said ankyrin repeat domain with binding specificity for human serum albumin comprises an amino acid sequence that is at least 98% identical to any one of SEQ ID NOs: 5 to 7. In one embodiment, said ankyrin repeat domain with binding specificity for human serum albumin comprises an amino acid sequence that is at least 99% identical to any one of SEQ ID NOs: 5 to 7. In one embodiment, any amino acid sequence differences between said ankyrin repeat domain with binding specificity for human serum albumin and any one of SEQ ID NOs: 5 to 7 represent amino acid substitutions in framework positions. In one embodiment, said ankyrin repeat domain with binding specificity for human serum albumin comprises an amino acid sequence that is 100% identical to any one of SEQ ID NOs: 5 to 7.

In another aspect, the invention relates to such a conjugate or pharmaceutically acceptable salt thereof, wherein said ankyrin repeat domain with binding specificity for human serum albumin comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 5. In one embodiment, any amino acid sequence differences between said ankyrin repeat domain with binding specificity for human serum albumin and SEQ ID NO: 5 represent amino acid substitutions in framework positions. In one embodiment, said ankyrin repeat domain with binding specificity for human serum albumin comprises an amino acid sequence that is 100% identical to SEQ ID NO: 5.

In another aspect, the invention relates to such a conjugate or pharmaceutically acceptable salt thereof, wherein said ankyrin repeat domain with binding specificity for human serum albumin comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 6. In one embodiment, any amino acid sequence differences between said ankyrin repeat domain with binding specificity for human serum albumin and SEQ ID NO: 6 represent amino acid substitutions in framework positions. In one embodiment, said ankyrin repeat domain with binding specificity for human serum albumin comprises an amino acid sequence that is 100% identical to SEQ ID NO: 6.

In another aspect, the invention relates to such a conjugate or pharmaceutically acceptable salt thereof, wherein said ankyrin repeat domain with binding specificity for human serum albumin comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 7. Thus, in one embodiment, said ankyrin repeat domain with binding specificity for human serum albumin comprises an amino acid sequence that is at least 80% identical to SEQ ID NO: 7. In one embodiment, said ankyrin repeat domain with binding specificity for human serum albumin comprises an amino acid sequence that is at least 85% identical to SEQ ID NO: 7. In one embodiment, said ankyrin repeat domain with binding specificity for human serum albumin comprises an amino acid sequence that is at least 88% identical to SEQ ID NO: 7. In one embodiment, said ankyrin repeat domain with binding specificity for human serum albumin comprises an amino acid sequence that is at least 90% identical to SEQ ID NO: 7. In one embodiment, said ankyrin repeat domain with binding specificity for human serum albumin comprises an amino acid sequence that is at least 91% identical to SEQ ID NO: 7. In one embodiment, said ankyrin repeat domain with binding specificity for human serum albumin comprises an amino acid sequence that is at least 92% identical to SEQ ID NO: 7. In one embodiment, said ankyrin repeat domain with binding specificity for human serum albumin comprises an amino acid sequence that is at least 93% identical to SEQ ID NO: 7. In one embodiment, said ankyrin repeat domain with binding specificity for human serum albumin comprises an amino acid sequence that is at least 94% identical to SEQ ID NO: 7. In one embodiment, said ankyrin repeat domain with binding specificity for human serum albumin comprises an amino acid sequence that is at least 95% identical to SEQ ID NO: 7. In one embodiment, said ankyrin repeat domain with binding specificity for human serum albumin comprises an amino acid sequence that is at least 96% identical to SEQ ID NO: 7. In one embodiment, said ankyrin repeat domain with binding specificity for human serum albumin comprises an amino acid sequence that is at least 97% identical to SEQ ID NO: 7. In one embodiment, said ankyrin repeat domain with binding specificity for human serum albumin comprises an amino acid sequence that is at least 98% identical to SEQ ID NO: 7. In one embodiment, said ankyrin repeat domain with binding specificity for human serum albumin comprises an amino acid sequence that is at least 99% identical to SEQ ID NO: 7. In one embodiment, said ankyrin repeat domain with binding specificity for human serum albumin comprises an amino acid sequence that is 100% identical to SEQ ID NO: 7. In one embodiment, any amino acid sequence differences between said ankyrin repeat domain with binding specificity for human serum albumin and SEQ ID NO: 7 represent amino acid substitutions in framework positions.

In another aspect, the invention relates to such a conjugate or pharmaceutically acceptable salt thereof, wherein said conjugate further comprises a peptide linker, and wherein said ankyrin repeat domain with binding specificity for human serum albumin is covalently connected to said ankyrin repeat domain with binding specificity for DLL3 by said peptide linker. Different peptide linkers are known in the art. Examples of peptide linkers include PT-rich linkers and GS-rich linkers. Peptide linkers can have different lengths. Thus, in one embodiment, said peptide linker comprises the amino acid sequence of any one of SEQ ID NOs: 8 or 9, or a variant thereof. In one embodiment, said peptide linker comprises the amino acid sequence of SEQ ID NO: 8 or a variant thereof. In one embodiment, said conjugate has the formula: H-L-D-Ch-R, wherein H is said half-life extending moiety, L is said peptide linker, D is said ankyrin repeat domain with binding specificity for DLL3, Ch is said chelator, and R is said radionuclide. In another embodiment, said conjugate has the formula: D-L-H-Ch-R, wherein H is said half-life extending moiety, L is said peptide linker, D is said ankyrin repeat domain with binding specificity for DLL3, Ch is said chelator, and R is said radionuclide. In one embodiment, said conjugate has the formula: H-L-D-T-Ch-R, wherein H is said half-life extending moiety, L is said peptide linker, D is said ankyrin repeat domain with binding specificity for DLL3, T is said tag, Ch is said chelator, and R is said radionuclide. In another embodiment, said conjugate has the formula: D-L-H-T-Ch-R, wherein H is said half-life extending moiety, L is said peptide linker, D is said ankyrin repeat domain with binding specificity for DLL3, T is said tag, Ch is said chelator, and R is said radionuclide. In one embodiment, said conjugate has the formula: H-L-D-Co-Ch-R, wherein H is said half-life extending moiety, L is said peptide linker, D is said ankyrin repeat domain with binding specificity for DLL3, Co is said connector, Ch is said chelator, and R is said radionuclide. In another embodiment, said conjugate has the formula: H-L-D-T-Co-Ch-R, wherein H is said half-life extending moiety, L is said peptide linker, D is said ankyrin repeat domain with binding specificity for DLL3, T is said tag, Co is said connector, Ch is said chelator, and R is said radionuclide. In another embodiment, said conjugate has the formula: D-L-H-Co-Ch-R, wherein H is said half-life extending moiety, L is said peptide linker, D is said ankyrin repeat domain with binding specificity for DLL3, Co is said connector, Ch is said chelator, and R is said radionuclide. In another embodiment, said conjugate has the formula: D-L-H-T-Co-Ch-R, wherein H is said half-life extending moiety, L is said peptide linker, D is said ankyrin repeat domain with binding specificity for DLL3, T is said tag, Co is said connector, Ch is said chelator, and R is said radionuclide.

In another aspect, the invention relates to such a conjugate or pharmaceutically acceptable salt thereof, wherein said conjugate comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 11 to 14. Said amino acid sequence comprised in said conjugate comprises an ankyrin repeat domain with binding specificity for serum albumin at the N-terminal side and an ankyrin repeat domain with binding specificity for human DLL3 at the C-terminal side, and a peptide linker that connects said two ankyrin repeat domains. Thus, in one embodiment, said conjugate comprises an amino acid sequence that is at least 80% identical to any one of SEQ ID NOs: 11 to 14. In one embodiment, said conjugate comprises an amino acid sequence that is at least 85% identical to any one of SEQ ID NOs: 11 to 14. In one embodiment, said conjugate comprises an amino acid sequence that is at least 88% identical to any one of SEQ ID NOs: 11 to 14. In one embodiment, said conjugate comprises an amino acid sequence that is at least 90% identical to any one of SEQ ID NOs: 11 to 14. In one embodiment, said conjugate comprises an amino acid sequence that is at least 91% identical to any one of SEQ ID NOs: 11 to 14. In one embodiment, said conjugate comprises an amino acid sequence that is at least 92% identical to any one of SEQ ID NOs: 11 to 14. In one embodiment, said conjugate comprises an amino acid sequence that is at least 93% identical to any one of SEQ ID NOs: 11 to 14. In one embodiment, said conjugate comprises an amino acid sequence that is at least 94% identical to any one of SEQ ID NOs: 11 to 14. In one embodiment, said conjugate comprises an amino acid sequence that is at least 95% identical to any one of SEQ ID NOs: 11 to 14. In one embodiment, said conjugate comprises an amino acid sequence that is at least 96% identical to any one of SEQ ID NOs: 11 to 14. In one embodiment, said conjugate comprises an amino acid sequence that is at least 97% identical to any one of SEQ ID NOs: 11 to 14. In one embodiment, said conjugate comprises an amino acid sequence that is at least 98% identical to any one of SEQ ID NOs: 11 to 14. In one embodiment, said conjugate comprises an amino acid sequence that is at least 99% identical to any one of SEQ ID NOs: 11 to 14. In one embodiment, any amino acid sequence differences between said amino acid sequence comprised in said conjugate and said any one of SEQ ID NOs: 11 to 14 represent amino acid substitutions in framework positions of said two ankyrin repeat domains and/or in said peptide linker. In one embodiment, said conjugate comprises an amino acid sequence that is 100% identical to any one of SEQ ID NOs: 11 to 14. In one embodiment, said conjugate comprises said amino acid sequence described in any of the above embodiments, wherein said amino acid sequence is covalently bound at its N-terminal end to Glycine-Serine (GS). In one embodiment, said conjugate comprises said amino acid sequence described in any of the above embodiments, wherein said amino acid sequence is covalently bound at its C-terminal end to the amino acid sequence of SEQ ID NO: 10.

In another aspect, the invention relates to such a conjugate or pharmaceutically acceptable salt thereof, wherein said conjugate comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 11. Thus, in one embodiment, said conjugate comprises an amino acid sequence that is at least 80% identical to SEQ ID NO: 11. In one embodiment, said conjugate comprises an amino acid sequence that is at least 81% identical to SEQ ID NO: 11. In one embodiment, said conjugate comprises an amino acid sequence that is at least 82% identical to SEQ ID NO: 11. In one embodiment, said conjugate comprises an amino acid sequence that is at least 83% identical to SEQ ID NO: 11. In one embodiment, said conjugate comprises an amino acid sequence that is at least 84% identical to SEQ ID NO: 11. In one embodiment, said conjugate comprises an amino acid sequence that is at least 85% identical to SEQ ID NO: 11. In one embodiment, said conjugate comprises an amino acid sequence that is at least 86% identical to SEQ ID NO: 11. In one embodiment, said conjugate comprises an amino acid sequence that is at least 87% identical to SEQ ID NO: 11. In one embodiment, said conjugate comprises an amino acid sequence that is at least 88% identical to SEQ ID NO: 11. In one embodiment, said conjugate comprises an amino acid sequence that is at least 89% identical to SEQ ID NO: 11. In one embodiment, said conjugate comprises an amino acid sequence that is at least 90% identical to SEQ ID NO: 11. In one embodiment, said conjugate comprises an amino acid sequence that is at least 91% identical to SEQ ID NO: 11. In one embodiment, said conjugate comprises an amino acid sequence that is at least 92% identical to SEQ ID NO: 11. In one embodiment, said conjugate comprises an amino acid sequence that is at least 93% identical SEQ ID NO: 11. In one embodiment, said conjugate comprises an amino acid sequence that is at least 94% identical to SEQ ID NO: 11. In one embodiment, said conjugate comprises an amino acid sequence that is at least 95% identical to SEQ ID NO: 11. In one embodiment, said conjugate comprises an amino acid sequence that is at least 96% identical to SEQ ID NO: 11. In one embodiment, said conjugate comprises an amino acid sequence that is at least 97% identical to SEQ ID NO: 11. In one embodiment, said conjugate comprises an amino acid sequence that is at least 98% identical to SEQ ID NO: 11. In one embodiment, said conjugate comprises an amino acid sequence that is at least 99% identical to SEQ ID NO: 11. In one embodiment, any amino acid sequence differences between said amino acid sequence comprised in said conjugate and SEQ ID NO: 11 represent amino acid substitutions in framework positions of said two ankyrin repeat domains and/or in said peptide linker. In one embodiment, said conjugate comprises an amino acid sequence that is 100% identical to SEQ ID NO: 11. In one embodiment, said conjugate comprises said amino acid sequence described in any of the above embodiments, wherein said amino acid sequence is covalently bound at its N-terminal end to Glycine-Serine (GS). In one embodiment, said conjugate comprises said amino acid sequence described in any of the above embodiments, wherein said amino acid sequence is covalently bound at its C-terminal end to the amino acid sequence of SEQ ID NO: 10.

In another aspect, the invention relates to such a conjugate or pharmaceutically acceptable salt thereof, wherein said conjugate comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 12. Thus, in one embodiment, said conjugate comprises an amino acid sequence that is at least 80% identical to SEQ ID NO: 12. In one embodiment, said conjugate comprises an amino acid sequence that is at least 85% identical to SEQ ID NO: 12. In one embodiment, said conjugate comprises an amino acid sequence that is at least 88% identical to SEQ ID NO: 12. In one embodiment, said conjugate comprises an amino acid sequence that is at least 90% identical to SEQ ID NO: 12. In one embodiment, said conjugate comprises an amino acid sequence that is at least 91% identical to SEQ ID NO: 12. In one embodiment, said conjugate comprises an amino acid sequence that is at least 92% identical to SEQ ID NO: 12. In one embodiment, said conjugate comprises an amino acid sequence that is at least 93% identical SEQ ID NO: 12. In one embodiment, said conjugate comprises an amino acid sequence that is at least 94% identical to SEQ ID NO: 12. In one embodiment, said conjugate comprises an amino acid sequence that is at least 95% identical to SEQ ID NO: 12. In one embodiment, said conjugate comprises an amino acid sequence that is at least 96% identical to SEQ ID NO: 12. In one embodiment, said conjugate comprises an amino acid sequence that is at least 97% identical to SEQ ID NO: 12. In one embodiment, said conjugate comprises an amino acid sequence that is at least 98% identical to SEQ ID NO: 12. In one embodiment, said conjugate comprises an amino acid sequence that is at least 99% identical to SEQ ID NO: 12. In one embodiment, any amino acid sequence differences between said amino acid sequence comprised in said conjugate and SEQ ID NO: 12 represent amino acid substitutions in framework positions of said two ankyrin repeat domains and/or in said peptide linker. In one embodiment, said conjugate comprises an amino acid sequence that is 100% identical to SEQ ID NO: 12. In one embodiment, said conjugate comprises said amino acid sequence described in any of the above embodiments, wherein said amino acid sequence is covalently bound at its N-terminal end to Glycine-Serine (GS). In one embodiment, said conjugate comprises said amino acid sequence described in any of the above embodiments, wherein said amino acid sequence is covalently bound at its C-terminal end to the amino acid sequence of SEQ ID NO: 10.

In another aspect, the invention relates to such a conjugate or pharmaceutically acceptable salt thereof, wherein said conjugate comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 13. Thus, in one embodiment, said conjugate comprises an amino acid sequence that is at least 80% identical to SEQ ID NO: 13. In one embodiment, said conjugate comprises an amino acid sequence that is at least 85% identical to SEQ ID NO: 13. In one embodiment, said conjugate comprises an amino acid sequence that is at least 88% identical to SEQ ID NO: 13. In one embodiment, said conjugate comprises an amino acid sequence that is at least 90% identical to SEQ ID NO: 13. In one embodiment, said conjugate comprises an amino acid sequence that is at least 91% identical to SEQ ID NO: 13. In one embodiment, said conjugate comprises an amino acid sequence that is at least 92% identical to SEQ ID NO: 13. In one embodiment, said conjugate comprises an amino acid sequence that is at least 93% identical SEQ ID NO: 13. In one embodiment, said conjugate comprises an amino acid sequence that is at least 94% identical to SEQ ID NO: 13. In one embodiment, said conjugate comprises an amino acid sequence that is at least 95% identical to SEQ ID NO: 13. In one embodiment, said conjugate comprises an amino acid sequence that is at least 96% identical to SEQ ID NO: 13. In one embodiment, said conjugate comprises an amino acid sequence that is at least 97% identical to SEQ ID NO: 13. In one embodiment, said conjugate comprises an amino acid sequence that is at least 98% identical to SEQ ID NO: 13. In one embodiment, said conjugate comprises an amino acid sequence that is at least 99% identical to SEQ ID NO: 13. In one embodiment, any amino acid sequence differences between said amino acid sequence comprised in said conjugate and SEQ ID NO: 13 represent amino acid substitutions in framework positions of said two ankyrin repeat domains and/or in said peptide linker. In one embodiment, said conjugate comprises an amino acid sequence that is 100% identical to SEQ ID NO: 13. In one embodiment, said conjugate comprises said amino acid sequence described in any of the above embodiments, wherein said amino acid sequence is covalently bound at its N-terminal end to Glycine-Serine (GS). In one embodiment, said conjugate comprises said amino acid sequence described in any of the above embodiments, wherein said amino acid sequence is covalently bound at its C-terminal end to the amino acid sequence of SEQ ID NO: 10.

In another aspect, the invention relates to such a conjugate or pharmaceutically acceptable salt thereof, wherein said conjugate comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 14. Thus, in one embodiment, said conjugate comprises an amino acid sequence that is at least 80% identical to SEQ ID NO: 14. In one embodiment, said conjugate comprises an amino acid sequence that is at least 85% identical to SEQ ID NO: 14. In one embodiment, said conjugate comprises an amino acid sequence that is at least 88% identical to SEQ ID NO: 14. In one embodiment, said conjugate comprises an amino acid sequence that is at least 90% identical to SEQ ID NO: 14. In one embodiment, said conjugate comprises an amino acid sequence that is at least 91% identical to SEQ ID NO: 14. In one embodiment, said conjugate comprises an amino acid sequence that is at least 92% identical to SEQ ID NO: 14. In one embodiment, said conjugate comprises an amino acid sequence that is at least 93% identical SEQ ID NO: 14. In one embodiment, said conjugate comprises an amino acid sequence that is at least 94% identical to SEQ ID NO: 14. In one embodiment, said conjugate comprises an amino acid sequence that is at least 95% identical to SEQ ID NO: 14. In one embodiment, said conjugate comprises an amino acid sequence that is at least 96% identical to SEQ ID NO: 14. In one embodiment, said conjugate comprises an amino acid sequence that is at least 97% identical to SEQ ID NO: 14. In one embodiment, said conjugate comprises an amino acid sequence that is at least 98% identical to SEQ ID NO: 14. In one embodiment, said conjugate comprises an amino acid sequence that is at least 99% identical to SEQ ID NO: 14. In one embodiment, any amino acid sequence differences between said amino acid sequence comprised in said conjugate and SEQ ID NO: 14 represent amino acid substitutions in framework positions of said two ankyrin repeat domains and/or in said peptide linker. In one embodiment, said conjugate comprises an amino acid sequence that is 100% identical to SEQ ID NO: 14. In one embodiment, said conjugate comprises said amino acid sequence described in any of the above embodiments, wherein said amino acid sequence is covalently bound at its N-terminal end to Glycine-Serine (GS). In one embodiment, said conjugate comprises said amino acid sequence described in any of the above embodiments, wherein said amino acid sequence is covalently bound at its C-terminal end to the amino acid sequence of SEQ ID NO: 10.

In another main aspect, the invention relates to a conjugate or pharmaceutically acceptable salt thereof, the conjugate comprising (i) an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 15 to 18, (ii) a chelator, and (iii) a radionuclide, wherein said ankyrin repeat protein has binding specificity for DLL3 and for human serum albumin, wherein said chelator is covalently connected to said ankyrin repeat protein, wherein said radionuclide is bound to said chelator, and wherein said radionuclide is Pb-212 or Pb-203. An amino acid sequence of any one of SEQ ID NOs: 15 to 18 comprises an ankyrin repeat domain with binding specificity for human serum albumin and an ankyrin repeat domain with binding specificity for human DLL3, wherein said ankyrin repeat domains are connected by a peptide linker, wherein said amino acid sequence comprises a Glycine-Serine (GS) at its N-terminus, and wherein said amino acid sequence comprises a Cysteine-containing tag at its C-terminus. In one embodiment, said radionuclide is Pb-212. In one embodiment, said radionuclide is Pb-203.

In another aspect, the invention relates to a conjugate or pharmaceutically acceptable salt thereof, the conjugate comprising (i) an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 15 to 18, (ii) a connector, (iii) a chelator, and (iv) a radionuclide, wherein said ankyrin repeat protein has binding specificity for DLL3 and for human serum albumin, wherein said connector is covalently connected to said ankyrin repeat protein, wherein said chelator is covalently connected to said connector, wherein said radionuclide is bound to said chelator, and wherein said radionuclide is Pb-212 or Pb-203. In one embodiment, said radionuclide is Pb-212. In one embodiment, said radionuclide is Pb-203.

In another aspect, the invention relates to such a conjugate or pharmaceutically acceptable salt thereof, wherein said ankyrin repeat protein with binding specificity for DLL3 and for human serum albumin comprises an amino acid sequence that is at least 80% identical to any one of SEQ ID NOs: 15 to 18. In one embodiment, said ankyrin repeat protein comprises an amino acid sequence that is at least 85% identical to any one of SEQ ID NOs: 15 to 18. In one embodiment, said ankyrin repeat protein comprises an amino acid sequence that is at least 88% identical to any one of SEQ ID NOs: 15 to 18. In one embodiment, said ankyrin repeat protein comprises an amino acid sequence that is at least 90% identical to any one of SEQ ID NOs: 15 to 18. In one embodiment, said ankyrin repeat protein comprises an amino acid sequence that is at least 91% identical to any one of SEQ ID NOs: 15 to 18. In one embodiment, said ankyrin repeat protein comprises an amino acid sequence that is at least 92% identical to any one of SEQ ID NOs: 15 to 18. In one embodiment, said ankyrin repeat protein comprises an amino acid sequence that is at least 93% identical to any one of SEQ ID NOs: 15 to 18. In one embodiment, said ankyrin repeat protein comprises an amino acid sequence that is at least 94% identical to any one of SEQ ID NOs: 15 to 18. In one embodiment, said ankyrin repeat protein comprises an amino acid sequence that is at least 95% identical to any one of SEQ ID NOs: 15 to 18. In one embodiment, said ankyrin repeat protein comprises an amino acid sequence that is at least 96% identical to any one of SEQ ID NOs: 15 to 18. In one embodiment, said ankyrin repeat protein comprises an amino acid sequence that is at least 97% identical to any one of SEQ ID NOs: 15 to 18. In one embodiment, said ankyrin repeat protein comprises an amino acid sequence that is at least 98% identical to any one of SEQ ID NOs: 15 to 18. In one embodiment, said ankyrin repeat protein comprises an amino acid sequence that is at least 99% identical to any one of SEQ ID NOs: 15 to 18. In one embodiment, any amino acid sequence differences between said amino acid sequence comprised in said ankyrin repeat protein and said any one of SEQ ID NOs: 15 to 18 represent amino acid substitutions in positions other than positions of potential target interaction residues of ankyrin repeat domains. In one embodiment, any amino acid sequence differences between said amino acid sequence comprised in said ankyrin repeat protein and said any one of SEQ ID NOs: 15 to 18 represent amino acid substitutions in (i) framework positions of ankyrin repeat domain(s), (ii) a peptide linker, (iii) a Glycine-Serine (GS), and/or (iv) a Cysteine-containing tag. In one embodiment, the C-terminal Cysteine of said any one of SEQ ID NOs: 15 to 18 is not substituted with another amino acid. In one embodiment, said ankyrin repeat protein comprises an amino acid sequence that is 100% identical to any one of SEQ ID NOs: 15 to 18.

In another aspect, the invention relates to such a conjugate or pharmaceutically acceptable salt thereof, wherein said ankyrin repeat protein with binding specificity for DLL3 and for human serum albumin comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 15. Thus, in one embodiment, said ankyrin repeat protein comprises an amino acid sequence that is at least 80% identical to SEQ ID NO: 15. In one embodiment, said ankyrin repeat protein comprises an amino acid sequence that is at least 81% identical to SEQ ID NO: 15. In one embodiment, said ankyrin repeat protein comprises an amino acid sequence that is at least 82% identical to SEQ ID NO: 15. In one embodiment, said ankyrin repeat protein comprises an amino acid sequence that is at least 83% identical to SEQ ID NO: 15. In one embodiment, said ankyrin repeat protein comprises an amino acid sequence that is at least 84% identical to SEQ ID NO: 15. In one embodiment, said ankyrin repeat protein comprises an amino acid sequence that is at least 85% identical to SEQ ID NO: 15. In one embodiment, said ankyrin repeat protein comprises an amino acid sequence that is at least 86% identical to SEQ ID NO: 15. In one embodiment, said ankyrin repeat protein comprises an amino acid sequence that is at least 87% identical to SEQ ID NO: 15. In one embodiment, said ankyrin repeat protein comprises an amino acid sequence that is at least 88% identical to SEQ ID NO: 15. In one embodiment, said ankyrin repeat protein comprises an amino acid sequence that is at least 89% identical to SEQ ID NO: 15. In one embodiment, said ankyrin repeat protein comprises an amino acid sequence that is at least 90% identical to SEQ ID NO: 15. In one embodiment, said ankyrin repeat protein comprises an amino acid sequence that is at least 91% identical to SEQ ID NO: 15. In one embodiment, said ankyrin repeat protein comprises an amino acid sequence that is at least 92% identical to SEQ ID NO: 15. In one embodiment, said ankyrin repeat protein comprises an amino acid sequence that is at least 93% identical SEQ ID NO: 15. In one embodiment, said ankyrin repeat protein comprises an amino acid sequence that is at least 94% identical to SEQ ID NO: 15. In one embodiment, said ankyrin repeat protein comprises an amino acid sequence that is at least 95% identical to SEQ ID NO: 15. In one embodiment, said ankyrin repeat protein comprises an amino acid sequence that is at least 96% identical to SEQ ID NO: 15. In one embodiment, said ankyrin repeat protein comprises an amino acid sequence that is at least 97% identical to SEQ ID NO: 15. In one embodiment, said ankyrin repeat protein comprises an amino acid sequence that is at least 98% identical to SEQ ID NO: 15. In one embodiment, said ankyrin repeat protein comprises an amino acid sequence that is at least 99% identical to SEQ ID NO: 15. In one embodiment, any amino acid sequence differences between said amino acid sequence comprised in said ankyrin repeat protein and SEQ ID NO: 15 represent amino acid substitutions in positions other than positions of potential target interaction residues of ankyrin repeat domains. In one embodiment, any amino acid sequence differences between said amino acid sequence comprised in said ankyrin repeat protein and SEQ ID NO: 15 represent amino acid substitutions in (i) framework positions of ankyrin repeat domain(s), (ii) a peptide linker, (iii) a Glycine-Serine (GS), and/or (iv) a Cysteine-containing tag. In one embodiment, the C-terminal Cysteine of SEQ ID NO: 15 is not substituted with another amino acid. In one embodiment, said ankyrin repeat protein comprises an amino acid sequence that is 100% identical to SEQ ID NO: 15.

In another aspect, the invention relates to such a conjugate or pharmaceutically acceptable salt thereof, wherein said ankyrin repeat protein with binding specificity for DLL3 and for human serum albumin comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 16. Thus, in one embodiment, said ankyrin repeat protein comprises an amino acid sequence that is at least 80% identical to SEQ ID NO: 16. In one embodiment, said ankyrin repeat protein comprises an amino acid sequence that is at least 85% identical to SEQ ID NO: 16. In one embodiment, said ankyrin repeat protein comprises an amino acid sequence that is at least 88% identical to SEQ ID NO: 16. In one embodiment, said ankyrin repeat protein comprises an amino acid sequence that is at least 90% identical to SEQ ID NO: 16. In one embodiment, said ankyrin repeat protein comprises an amino acid sequence that is at least 91% identical to SEQ ID NO: 16. In one embodiment, said ankyrin repeat protein comprises an amino acid sequence that is at least 92% identical to SEQ ID NO: 16. In one embodiment, said ankyrin repeat protein comprises an amino acid sequence that is at least 93% identical SEQ ID NO: 16.

In one embodiment, said ankyrin repeat protein comprises an amino acid sequence that is at least 94% identical to SEQ ID NO: 16. In one embodiment, said ankyrin repeat protein comprises an amino acid sequence that is at least 95% identical to SEQ ID NO: 16. In one embodiment, said ankyrin repeat protein comprises an amino acid sequence that is at least 96% identical to SEQ ID NO: 16. In one embodiment, said ankyrin repeat protein comprises an amino acid sequence that is at least 97% identical to SEQ ID NO: 16. In one embodiment, said ankyrin repeat protein comprises an amino acid sequence that is at least 98% identical to SEQ ID NO: 16. In one embodiment, said ankyrin repeat protein comprises an amino acid sequence that is at least 99% identical to SEQ ID NO: 16. In one embodiment, any amino acid sequence differences between said amino acid sequence comprised in said ankyrin repeat protein and SEQ ID NO: 16 represent amino acid substitutions in positions other than positions of potential target interaction residues of ankyrin repeat domains. In one embodiment, any amino acid sequence differences between said amino acid sequence comprised in said ankyrin repeat protein and SEQ ID NO: 16 represent amino acid substitutions in (i) framework positions of ankyrin repeat domain(s), (ii) a peptide linker, (iii) a Glycine-Serine (GS), and/or (iv) a Cysteine-containing tag. In one embodiment, the C-terminal Cysteine of SEQ ID NO: 16 is not substituted with another amino acid. In one embodiment, said ankyrin repeat protein comprises an amino acid sequence that is 100% identical to SEQ ID NO: 16.

In another aspect, the invention relates to such a conjugate or pharmaceutically acceptable salt thereof, wherein said ankyrin repeat protein with binding specificity for DLL3 and for human serum albumin comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 17. Thus, in one embodiment, said ankyrin repeat protein comprises an amino acid sequence that is at least 80% identical to SEQ ID NO: 17. In one embodiment, said ankyrin repeat protein comprises an amino acid sequence that is at least 85% identical to SEQ ID NO: 17. In one embodiment, said ankyrin repeat protein comprises an amino acid sequence that is at least 88% identical to SEQ ID NO: 17. In one embodiment, said ankyrin repeat protein comprises an amino acid sequence that is at least 90% identical to SEQ ID NO: 17. In one embodiment, said ankyrin repeat protein comprises an amino acid sequence that is at least 91% identical to SEQ ID NO: 17. In one embodiment, said ankyrin repeat protein comprises an amino acid sequence that is at least 92% identical to SEQ ID NO: 17. In one embodiment, said ankyrin repeat protein comprises an amino acid sequence that is at least 93% identical SEQ ID NO: 17. In one embodiment, said ankyrin repeat protein comprises an amino acid sequence that is at least 94% identical to SEQ ID NO: 17. In one embodiment, said ankyrin repeat protein comprises an amino acid sequence that is at least 95% identical to SEQ ID NO: 17. In one embodiment, said ankyrin repeat protein comprises an amino acid sequence that is at least 96% identical to SEQ ID NO: 17. In one embodiment, said ankyrin repeat protein comprises an amino acid sequence that is at least 97% identical to SEQ ID NO: 17. In one embodiment, said ankyrin repeat protein comprises an amino acid sequence that is at least 98% identical to SEQ ID NO: 17. In one embodiment, said ankyrin repeat protein comprises an amino acid sequence that is at least 99% identical to SEQ ID NO: 17. In one embodiment, any amino acid sequence differences between said amino acid sequence comprised in said ankyrin repeat protein and SEQ ID NO: 17 represent amino acid substitutions in positions other than positions of potential target interaction residues of ankyrin repeat domains. In one embodiment, any amino acid sequence differences between said amino acid sequence comprised in said ankyrin repeat protein and SEQ ID NO: 17 represent amino acid substitutions in (i) framework positions of ankyrin repeat domain(s), (ii) a peptide linker, (iii) a Glycine-Serine (GS), and/or (iv) a Cysteine-containing tag. In one embodiment, the C-terminal Cysteine of SEQ ID NO: 17 is not substituted with another amino acid. In one embodiment, said ankyrin repeat protein comprises an amino acid sequence that is 100% identical to SEQ ID NO: 17.

In another aspect, the invention relates to such a conjugate or pharmaceutically acceptable salt thereof, wherein said ankyrin repeat protein with binding specificity for DLL3 and for human serum albumin comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 18. Thus, in one embodiment, said ankyrin repeat protein comprises an amino acid sequence that is at least 80% identical to SEQ ID NO: 18. In one embodiment, said ankyrin repeat protein comprises an amino acid sequence that is at least 85% identical to SEQ ID NO: 18. In one embodiment, said ankyrin repeat protein comprises an amino acid sequence that is at least 88% identical to SEQ ID NO: 18. In one embodiment, said ankyrin repeat protein comprises an amino acid sequence that is at least 90% identical to SEQ ID NO: 18. In one embodiment, said ankyrin repeat protein comprises an amino acid sequence that is at least 91% identical to SEQ ID NO: 18. In one embodiment, said ankyrin repeat protein comprises an amino acid sequence that is at least 92% identical to SEQ ID NO: 18. In one embodiment, said ankyrin repeat protein comprises an amino acid sequence that is at least 93% identical SEQ ID NO: 18. In one embodiment, said ankyrin repeat protein comprises an amino acid sequence that is at least 94% identical to SEQ ID NO: 18. In one embodiment, said ankyrin repeat protein comprises an amino acid sequence that is at least 95% identical to SEQ ID NO: 18. In one embodiment, said ankyrin repeat protein comprises an amino acid sequence that is at least 96% identical to SEQ ID NO: 18. In one embodiment, said ankyrin repeat protein comprises an amino acid sequence that is at least 97% identical to SEQ ID NO: 18. In one embodiment, said ankyrin repeat protein comprises an amino acid sequence that is at least 98% identical to SEQ ID NO: 18. In one embodiment, said ankyrin repeat protein comprises an amino acid sequence that is at least 99% identical to SEQ ID NO: 18. In one embodiment, any amino acid sequence differences between said amino acid sequence comprised in said ankyrin repeat protein and SEQ ID NO: 18 represent amino acid substitutions in positions other than positions of potential target interaction residues of ankyrin repeat domains. In one embodiment, any amino acid sequence differences between said amino acid sequence comprised in said ankyrin repeat protein and SEQ ID NO: 18 represent amino acid substitutions in (i) framework positions of ankyrin repeat domain(s), (ii) a peptide linker, (iii) a Glycine-Serine (GS), and/or (iv) a Cysteine-containing tag. In one embodiment, the C-terminal Cysteine of SEQ ID NO: 18 is not substituted with another amino acid. In one embodiment, said ankyrin repeat protein comprises an amino acid sequence that is 100% identical to SEQ ID NO: 18.

In another aspect, the invention relates to such a conjugate or pharmaceutically acceptable salt thereof, wherein said ankyrin repeat protein comprises a Cysteine, wherein said connector comprises maleimide or a derivative thereof, and wherein said Cysteine is covalently bound to said connector via a thioether bond. In one embodiment, said Cysteine is located at the C-terminal end of said amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 15 to 18.

In another aspect, the invention relates to such a conjugate or pharmaceutically acceptable salt thereof, wherein said ankyrin repeat protein has a melting temperature (Tm) of at least about 70° C., at least about 75° C., at least about 80° C., at least about 82° C., at least about 85° C., at least about 88° C., or at least about 90° C. Thus, in one embodiment, said ankyrin repeat protein has a melting temperature (Tm) of at least about 70° C. In one embodiment, said ankyrin repeat protein has a melting temperature (Tm) of at least about 75° C. In one embodiment, said ankyrin repeat protein has a melting temperature (Tm) of at least about 80° C. In one embodiment, said ankyrin repeat protein has a melting temperature (Tm) of at least about 82° C. In one embodiment, said ankyrin repeat protein has a melting temperature (Tm) of at least about 85° C. In one embodiment, said ankyrin repeat protein has a melting temperature (Tm) of at least about 88° C. In one embodiment, said ankyrin repeat protein has a melting temperature (Tm) of at least about 90° C. In one embodiment, said melting temperature (Tm) of said ankyrin repeat protein is determined in PBS. In one embodiment, said melting temperature (Tm) of said ankyrin repeat protein is determined by Circular Dichroism (CD) spectroscopy.

In another aspect, the invention relates to such a conjugate or pharmaceutically acceptable salt thereof, wherein said chelator comprises a 1,4,7,10-tetraazacyclododecane ring (PubChem CID 64963), or a derivative thereof. In one embodiment, said 1,4,7,10-tetraazacyclododecane ring comprises one or more side chains. In one embodiment, said one or more side chains are connected to one or more of the nitrogen atoms of said 1,4,7,10-tetraazacyclododecane ring. In one embodiment, said 1,4,7,10-tetraazacyclododecane ring comprises one, two, three or four side chains, wherein each of said side chains is connected to a nitrogen atom of said 1,4,7,10-tetraazacyclododecane ring. In one embodiment, at least one of said one or more side chains comprises a carboxyl group (—COOH) or an amide group (—CONH₂). In one embodiment, at least one of said one or more side chains comprises a —CH₂—COOH group or a —CH₂—CONH₂ group. In one embodiment, said 1,4,7,10-tetraazacyclododecane ring comprises four side chains, wherein each of said side chains is connected to a nitrogen atom of said 1,4,7,10-tetraazacyclododecane ring, and wherein each of said side chains comprises a carboxyl group (—COOH) or an amide group (—CONH₂). In one embodiment, said 1,4,7,10-tetraazacyclododecane ring comprises four side chains, wherein each of said side chains is connected to a nitrogen atom of said 1,4,7,10-tetraazacyclododecane ring, and wherein each of said side chains comprises a —CH₂—COOH group or a —CH₂—CONH₂ group. In one embodiment, said chelator is DOTA (1,4,7,10-tetraazacyclododecane-1,4,7,10-tetraacetic acid) or TCMC (1,4,7,10-Tetraazacyclododecane-1,4,7,10-tetraacetamide), or a derivative thereof. In one embodiment, said chelator is DOTA (1,4,7,10-tetraazacyclododecane-1,4,7,10-tetraacetic acid), or a derivative thereof. In one embodiment, said chelator is TCMC (1,4,7,10-Tetraazacyclododecane-1,4,7,10-tetraacetamide), or a derivative thereof. TCMC is also called DOTAM or DOTA-amide. Derivatives of TCMC include, for example, monoacid forms of TCMC. Thus, in one embodiment, said chelator comprises a 1,4,7,10-tetraazacyclododecane ring, wherein said 1,4,7,10-tetraazacyclododecane ring comprises four side chains, wherein each of said side chains is connected to a nitrogen atom of said 1,4,7,10-tetraazacyclododecane ring, and wherein one of said side chains comprises a —CH₂—COOH group and at least one of said side chains comprises a —CH₂—CONH₂ group.

In another aspect, the invention relates to such a conjugate or pharmaceutically acceptable salt thereof, wherein said chelator has a structure of Formula (I):

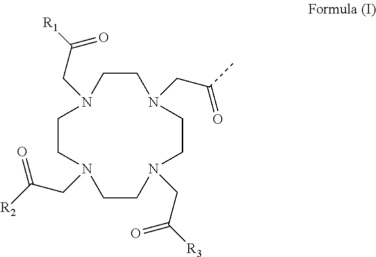

wherein R1, R2 and R3 are independently NH₂ or OH, and wherein the dotted line represents the covalent connection to said ankyrin repeat protein or said connector; or has a structure of Formula (III):

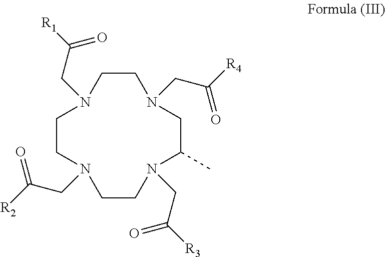

wherein R1, R2, R3 and R4 are independently NH₂ or OH, and wherein the dotted line represents the covalent connection to said ankyrin repeat protein or said connector.

In another aspect, the invention relates to such a conjugate or pharmaceutically acceptable salt thereof, wherein said chelator has a structure of Formula (I):

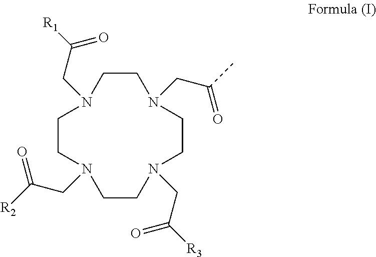

wherein R1, R2 and R3 are independently NH₂ or OH, and wherein the dotted line represents the covalent connection to said ankyrin repeat protein or said connector. In one embodiment, all three of R1, R2 and R3 in Formula (I) are OH. In one embodiment, one of R1, R2 and R3 in Formula (I) is NH₂ and two of R1, R2 and R3 in Formula (I) are OH. In one embodiment, two of R1, R2 and R3 in Formula (I) are NH₂ and one of R1, R2 and R3 in Formula (I) is OH. In one embodiment, all three of R1, R2 and R3 in Formula (I) are NH₂. In one embodiment, said chelator has a structure of Formula (II):

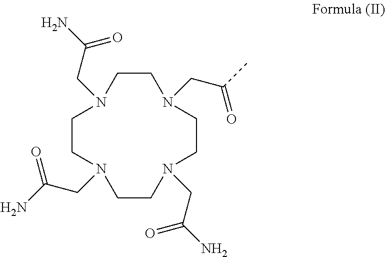

wherein the dotted line represents the covalent connection to said ankyrin repeat protein or said connector.

In another aspect, the invention relates to such a conjugate or pharmaceutically acceptable salt thereof, wherein said chelator has a structure of Formula (III):

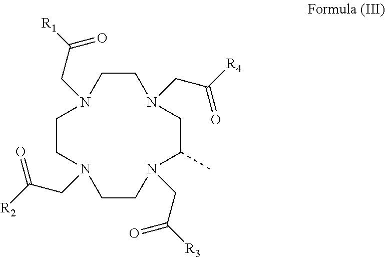

wherein R1, R2, R3 and R4 are independently NH₂ or OH, and wherein the dotted line represents the covalent connection to said ankyrin repeat protein or said connector. In one embodiment, all four of R1, R2, R3 and R4 in Formula (III) are OH. In one embodiment, one of R1, R2, R3 and R4 in Formula (III) is NH₂ and three of R1, R2, R3 and R4 in Formula (III) are OH. In one embodiment, two of R1, R2, R3 and R4 in Formula (III) are NH₂ and two of R1, R2, R3 and R4 in Formula (III) are OH. In one embodiment, three of R1, R2, R3 and R4 in Formula (III) are NH₂ and one of R1, R2, R3 and R4 in Formula (III) is OH. In one embodiment, all four of R1, R2, R3 and R4 in Formula (III) are NH₂. In one embodiment, said chelator has a structure of Formula (IV):

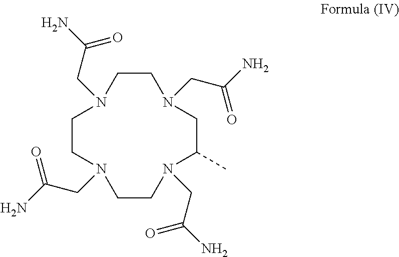 [SMILES: C[C@H]1CN(CC(N)=O)CCN(CC(N)=O)CCN(CC(N)=O)CCN1CC(N)=O]

wherein the dotted line represents the covalent connection to said ankyrin repeat protein or said connector.

In another main aspect, the invention relates to a conjugate or pharmaceutically acceptable salt thereof, the conjugate having a structure of Formula (VI):

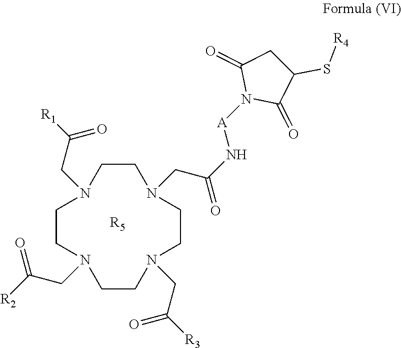

wherein R1, R2, and R3 are independently NH₂ or OH;
wherein A is CₐH_{b}N_{c}O_{d}, wherein a, b, c, and d are integers;
wherein R4 is an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 15 to 18, wherein said ankyrin repeat protein has binding specificity for DLL3 and for human serum albumin, and wherein said ankyrin repeat protein comprises a Cysteine;
and wherein R5 is a chelated radionuclide, wherein said radionuclide is Pb-212 or Pb-203;
or having a structure of Formula (VII):

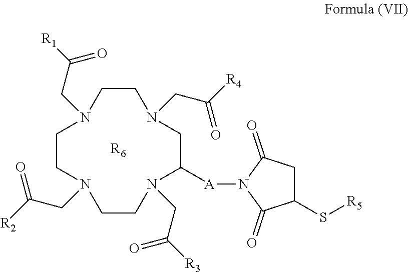

wherein R1, R2, R3 and R4 are independently NH₂ or OH;
wherein A is CₐH_{b}N_{c}Oₐ, wherein a, b, c, and d are integers;
wherein R5 is an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 15 to 18, wherein said ankyrin repeat protein has binding specificity for DLL3 and for human serum albumin, and wherein said ankyrin repeat protein comprises a Cysteine;
and wherein R6 is a chelated radionuclide, wherein said radionuclide is Pb-212 or Pb-203. In one embodiment, said Cysteine in R4 in Formula (VI) forms a thioether connecting said ankyrin repeat protein R4 with a maleimide ring and said Cysteine in R5 in Formula (VII) forms a thioether bond connecting said ankyrin repeat protein R5 with a maleimide ring.

In one embodiment, said R5 in Formula (VI) or said R6 in Formula (VII) is a chelated radionuclide, wherein said radionuclide is Pb-212. In one embodiment, said R5 in Formula (VI) or said R6 in Formula (VII) is a chelated radionuclide, wherein said radionuclide is Pb-203.

Thus, in one aspect, the invention relates to a conjugate or pharmaceutically acceptable salt thereof, the conjugate having a structure of Formula (VI):

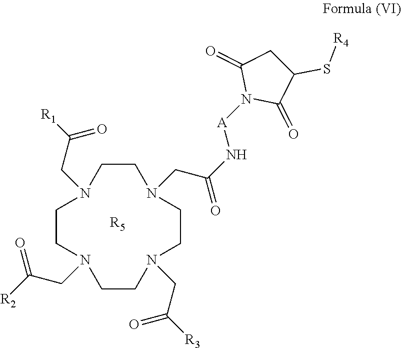

wherein R1, R2, and R3 are independently NH₂ or OH;
wherein A is CₐH_{b}N_{c}O_{d}, wherein a, b, c, and d are integers;
wherein R4 is an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 15 to 18, wherein said ankyrin repeat protein has binding specificity for DLL3 and for human serum albumin, and wherein said ankyrin repeat protein comprises a Cysteine;
and wherein R5 is a chelated radionuclide, wherein said radionuclide is Pb-212 or Pb-203. In one embodiment, said Cysteine in R4 in Formula (VI) forms a thioether bond connecting said ankyrin repeat protein R4 with a maleimide ring, wherein said maleimide ring connects A and R4. In one embodiment, said R5 in Formula (VI) is a chelated radionuclide, wherein said radionuclide is Pb-212. In one embodiment, said R5 in Formula (VI) is a chelated radionuclide, wherein said radionuclide is Pb-203. In one embodiment, all three of R1, R2 and R3 in Formula (VI) are OH. In one embodiment, one of R1, R2 and R3 in Formula (VI) is NH₂ and two of R1, R2 and R3 in Formula (VI) are OH. In one embodiment, two of R1, R2 and R3 in Formula (VI) are NH₂ and one of R1, R2 and R3 in Formula (VI) is OH. In one embodiment, all three of R1, R2 and R3 in Formula (VI) are NH₂. In one embodiment, said A in Formula (VI) is a hydrocarbon bridge. In one embodiment, said A in Formula (VI) is —CH₂—CH₂—. In one embodiment, all three of R1, R2 and R3 in Formula (VI) are NH₂ and said A in Formula (VI) is —CH₂—CH₂—.

Thus, in one aspect, the invention relates to a conjugate or pharmaceutically acceptable salt thereof, the conjugate having a structure of Formula (VII):

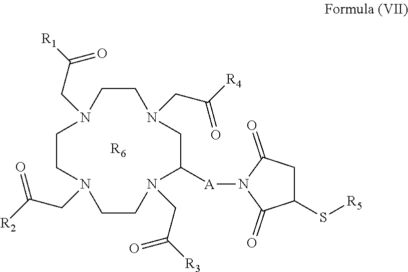

wherein R1, R2, R3 and R4 are independently NH₂ or OH;
wherein A is CₐH_{b}N_{c}O_{d}, wherein a, b, c, and d are integers;
wherein R5 is an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 15 to 18, wherein said ankyrin repeat protein has binding specificity for DLL3 and for human serum albumin, and wherein said ankyrin repeat protein comprises a Cysteine;
and wherein R6 is a chelated radionuclide, wherein said radionuclide is Pb-212 or Pb-203. In one embodiment, said Cysteine in R5 in Formula (VII) forms a thioether bond connecting said ankyrin repeat protein R5 with a maleimide ring, wherein said maleimide ring connects A and R5. In one embodiment, said R6 in Formula (VII) is a chelated radionuclide, wherein said radionuclide is Pb-212. In one embodiment, said R6 in Formula (VII) is a chelated radionuclide, wherein said radionuclide is Pb-203. In one embodiment, all four of R1, R2, R3 and R4 in Formula (VII) are OH. In one embodiment, one of R1, R2, R3 and R4 in Formula (VII) is NH₂ and three of R1, R2, R3 and R4 in Formula (VII) are OH. In one embodiment, two of R1, R2, R3 and R4 in Formula (VII) are NH₂ and two of R1, R2, R3 and R4 in Formula (VII) are OH. In one embodiment, three of R1, R2, R3 and R4 in Formula (VII) are NH₂ and one of R1, R2, R3 and R4 in Formula (VII) is OH. In one embodiment, all four of R1, R2, R3 and R4 in Formula (VII) are NH₂. In one embodiment, said A in Formula (VII) is a hydrocarbon bridge. In one embodiment, said A in Formula (VII) is —CH₂—CH₂—.

In another aspect, the invention relates to such a conjugate or pharmaceutically acceptable salt thereof, wherein said R4 in Formula (VI) or said R5 in Formula (VII) is an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 15 to 18. Thus, in one embodiment, said R4 in Formula (VI) or said R5 in Formula (VII) is an ankyrin repeat protein comprising an amino acid sequence that is at least 80% identical to any one of SEQ ID NOS: 15 to 18. In one embodiment, said R4 in Formula (VI) or said R5 in Formula (VII) is an ankyrin repeat protein comprising an amino acid sequence that is at least 85% identical to any one of SEQ ID NOS: 15 to 18. In one embodiment, said R4 in Formula (VI) or said R5 in Formula (VII) is an ankyrin repeat protein comprising an amino acid sequence that is at least 88% identical to any one of SEQ ID NOs: 15 to 18. In one embodiment, said R4 in Formula (VI) or said R5 in Formula (VII) is an ankyrin repeat protein comprising an amino acid sequence that is at least 90% identical to any one of SEQ ID NOs: 15 to 18. In one embodiment, said R4 in Formula (VI) or said R5 in Formula (VII) is an ankyrin repeat protein comprising an amino acid sequence that is at least 91% identical to any one of SEQ ID NOs: 15 to 18. In one embodiment, said R4 in Formula (VI) or said R5 in Formula (VII) is an ankyrin repeat protein comprising an amino acid sequence that is at least 92% identical to any one of SEQ ID NOs: 15 to 18. In one embodiment, said R4 in Formula (VI) or said R5 in Formula (VII) is an ankyrin repeat protein comprising an amino acid sequence that is at least 93% identical SEQ ID NO: 16. In one embodiment, said R4 in Formula (VI) or said R5 in Formula (VII) is an ankyrin repeat protein comprising an amino acid sequence that is at least 94% identical to any one of SEQ ID NOs: 15 to 18. In one embodiment, said R4 in Formula (VI) or said R5 in Formula (VII) is an ankyrin repeat protein comprising an amino acid sequence that is at least 95% identical to any one of SEQ ID NOs: 15 to 18. In one embodiment, said R4 in Formula (VI) or said R5 in Formula (VII) is an ankyrin repeat protein comprising an amino acid sequence that is at least 96% identical to any one of SEQ ID NOs: 15 to 18. In one embodiment, said R4 in Formula (VI) or said R5 in Formula (VII) is an ankyrin repeat protein comprising an amino acid sequence that is at least 97% identical to any one of SEQ ID NOs: 15 to 18. In one embodiment, said R4 in Formula (VI) or said R5 in Formula (VII) is an ankyrin repeat protein comprising an amino acid sequence that is at least 98% identical to any one of SEQ ID NOs: 15 to 18. In one embodiment, said R4 in Formula (VI) or said R5 in Formula (VII) is an ankyrin repeat protein comprising an amino acid sequence that is at least 99% identical to any one of SEQ ID NOs: 15 to 18. In one embodiment, any amino acid sequence differences between said amino acid sequence comprised in said ankyrin repeat protein (R4) in Formula (VI) or in said ankyrin repeat protein (R5) in Formula (VII) and said any one of SEQ ID NOs: 15 to 18 represent amino acid substitutions in positions other than positions of potential target interaction residues of ankyrin repeat domains. In one embodiment, any amino acid sequence differences between said amino acid sequence comprised in said ankyrin repeat protein (R4) in Formula (VI) or in said ankyrin repeat protein (R5) in Formula (VII) and said any one of SEQ ID NOs: 15 to 18 represent amino acid substitutions in (i) framework positions of ankyrin repeat domain(s), (ii) a peptide linker, (iii) a Glycine-Serine (GS), and/or (iv) a Cysteine-containing tag. In one embodiment, the C-terminal Cysteine of said any one of SEQ ID NOs: 15 to 18 is not substituted with another amino acid. In one embodiment, said amino acid sequence comprised in said ankyrin repeat protein (R4) in Formula (VI) or in said ankyrin repeat protein (R5) in Formula (VII) has a Cysteine at the C-terminal end. In one embodiment, said R4 in Formula (VI) or said R5 in Formula (VII) is an ankyrin repeat protein comprising an amino acid sequence that is 100% identical to any one of SEQ ID NOs: 15 to 18.

In another aspect, the invention relates to such a conjugate or pharmaceutically acceptable salt thereof, wherein said R4 in Formula (VI) or said R5 in Formula (VII) is an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 15. Thus, in one embodiment, said R4 in Formula (VI) or said R5 in Formula (VII) is an ankyrin repeat protein comprising an amino acid sequence that is at least 80% identical to SEQ ID NO: 15. In one embodiment, said R4 in Formula (VI) or said R5 in Formula (VII) is an ankyrin repeat protein comprising an amino acid sequence that is at least 81% identical to SEQ ID NO: 15. In one embodiment, said R4 in Formula (VI) or said R5 in Formula (VII) is an ankyrin repeat protein comprising an amino acid sequence that is at least 82% identical to SEQ ID NO: 15. In one embodiment, said R4 in Formula (VI) or said R5 in Formula (VII) is an ankyrin repeat protein comprising an amino acid sequence that is at least 83% identical to SEQ ID NO: 15. In one embodiment, said R4 in Formula (VI) or said R5 in Formula (VII) is an ankyrin repeat protein comprising an amino acid sequence that is at least 84% identical to SEQ ID NO: 15. In one embodiment, said R4 in Formula (VI) or said R5 in Formula (VII) is an ankyrin repeat protein comprising an amino acid sequence that is at least 85% identical to SEQ ID NO: 15. In one embodiment, said R4 in Formula (VI) or said R5 in Formula (VII) is an ankyrin repeat protein comprising an amino acid sequence that is at least 86% identical to SEQ ID NO: 15. In one embodiment, said R4 in Formula (VI) or said R5 in Formula (VII) is an ankyrin repeat protein comprising an amino acid sequence that is at least 87% identical to SEQ ID NO: 15. In one embodiment, said R4 in Formula (VI) or said R5 in Formula (VII) is an ankyrin repeat protein comprising an amino acid sequence that is at least 88% identical to SEQ ID NO: 15. In one embodiment, said R4 in Formula (VI) or said R5 in Formula (VII) is an ankyrin repeat protein comprising an amino acid sequence that is at least 89% identical to SEQ ID NO: 15. In one embodiment, said R4 in Formula (VI) or said R5 in Formula (VII) is an ankyrin repeat protein comprising an amino acid sequence that is at least 90% identical to SEQ ID NO: 15. In one embodiment, said R4 in Formula (VI) or said R5 in Formula (VII) is an ankyrin repeat protein comprising an amino acid sequence that is at least 91% identical to SEQ ID NO: 15. In one embodiment, said R4 in Formula (VI) or said R5 in Formula (VII) is an ankyrin repeat protein comprising an amino acid sequence that is at least 92% identical to SEQ ID NO: 15. In one embodiment, said R4 in Formula (VI) or said R5 in Formula (VII) is an ankyrin repeat protein comprising an amino acid sequence that is at least 93% identical SEQ ID NO: 15. In one embodiment, said R4 in Formula (VI) or said R5 in Formula (VII) is an ankyrin repeat protein comprising an amino acid sequence that is at least 94% identical to SEQ ID NO: 15. In one embodiment, said R4 in Formula (VI) or said R5 in Formula (VII) is an ankyrin repeat protein comprising an amino acid sequence that is at least 95% identical to SEQ ID NO: 15. In one embodiment, said R4 in Formula (VI) or said R5 in Formula (VII) is an ankyrin repeat protein comprising an amino acid sequence that is at least 96% identical to SEQ ID NO: 15. In one embodiment, said R4 in Formula (VI) or said R5 in Formula (VII) is an ankyrin repeat protein comprising an amino acid sequence that is at least 97% identical to SEQ ID NO: 15. In one embodiment, said R4 in Formula (VI) or said R5 in Formula (VII) is an ankyrin repeat protein comprising an amino acid sequence that is at least 98% identical to SEQ ID NO: 15. In one embodiment, said R4 in Formula (VI) or said R5 in Formula (VII) is an ankyrin repeat protein comprising an amino acid sequence that is at least 99% identical to SEQ ID NO: 15. In one embodiment, any amino acid sequence differences between said amino acid sequence comprised in said ankyrin repeat protein (R4) in Formula (VI) or in said ankyrin repeat protein (R5) in Formula (VII) and SEQ ID NO: 15 represent amino acid substitutions in positions other than positions of potential target interaction residues of ankyrin repeat domains. In one embodiment, any amino acid sequence differences between said amino acid sequence comprised in said ankyrin repeat protein (R4) in Formula (VI) or in said ankyrin repeat protein (R5) in Formula (VII) and SEQ ID NO: 15 represent amino acid substitutions in (i) framework positions of ankyrin repeat domain(s), (ii) a peptide linker, (iii) a Glycine-Serine (GS), and/or (iv) a Cysteine-containing tag. In one embodiment, the C-terminal Cysteine of SEQ ID NO: 15 is not substituted with another amino acid. In one embodiment, said amino acid sequence comprised in said ankyrin repeat protein (R4) in Formula (VI) or in said ankyrin repeat protein (R5) in Formula (VII) has a Cysteine at the C-terminal end. In one embodiment, said R4 in Formula (VI) or said R5 in Formula (VII) is an ankyrin repeat protein comprising an amino acid sequence that is 100% identical to SEQ ID NO: 15.

In another aspect, the invention relates to such a conjugate or pharmaceutically acceptable salt thereof, wherein said R4 in Formula (VI) or said R5 in Formula (VII) is an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 16. Thus, in one embodiment, said R4 in Formula (VI) or said R5 in Formula (VII) is an ankyrin repeat protein comprising an amino acid sequence that is at least 80% identical to SEQ ID NO: 16. In one embodiment, said R4 in Formula (VI) or said R5 in Formula (VII) is an ankyrin repeat protein comprising an amino acid sequence that is at least 85% identical to SEQ ID NO: 16. In one embodiment, said R4 in Formula (VI) or said R5 in Formula (VII) is an ankyrin repeat protein comprising an amino acid sequence that is at least 88% identical to SEQ ID NO: 16. In one embodiment, said R4 in Formula (VI) or said R5 in Formula (VII) is an ankyrin repeat protein comprising an amino acid sequence that is at least 90% identical to SEQ ID NO: 16. In one embodiment, said R4 in Formula (VI) or said R5 in Formula (VII) is an ankyrin repeat protein comprising an amino acid sequence that is at least 91% identical to SEQ ID NO: 16. In one embodiment, said R4 in Formula (VI) or said R5 in Formula (VII) is an ankyrin repeat protein comprising an amino acid sequence that is at least 92% identical to SEQ ID NO: 16. In one embodiment, said R4 in Formula (VI) or said R5 in Formula (VII) is an ankyrin repeat protein comprising an amino acid sequence that is at least 93% identical SEQ ID NO: 16. In one embodiment, said R4 in Formula (VI) or said R5 in Formula (VII) is an ankyrin repeat protein comprising an amino acid sequence that is at least 94% identical to SEQ ID NO: 16. In one embodiment, said R4 in Formula (VI) or said R5 in Formula (VII) is an ankyrin repeat protein comprising an amino acid sequence that is at least 95% identical to SEQ ID NO: 16. In one embodiment, said R4 in Formula (VI) or said R5 in Formula (VII) is an ankyrin repeat protein comprising an amino acid sequence that is at least 96% identical to SEQ ID NO: 16. In one embodiment, said R4 in Formula (VI) or said R5 in Formula (VII) is an ankyrin repeat protein comprising an amino acid sequence that is at least 97% identical to SEQ ID NO: 16. In one embodiment, said R4 in Formula (VI) or said R5 in Formula (VII) is an ankyrin repeat protein comprising an amino acid sequence that is at least 98% identical to SEQ ID NO: 16. In one embodiment, said R4 in Formula (VI) or said R5 in Formula (VII) is an ankyrin repeat protein comprising an amino acid sequence that is at least 99% identical to SEQ ID NO: 16. In one embodiment, any amino acid sequence differences between said amino acid sequence comprised in said ankyrin repeat protein (R4) in Formula (VI) or in said ankyrin repeat protein (R5) in Formula (VII) and SEQ ID NO: 16 represent amino acid substitutions in positions other than positions of potential target interaction residues of ankyrin repeat domains. In one embodiment, any amino acid sequence differences between said amino acid sequence comprised in said ankyrin repeat protein (R4) in Formula (VI) or in said ankyrin repeat protein (R5) in Formula (VII) and SEQ ID NO: 16 represent amino acid substitutions in (i) framework positions of ankyrin repeat domain(s), (ii) a peptide linker, (iii) a Glycine-Serine (GS), and/or (iv) a Cysteine-containing tag. In one embodiment, the C-terminal Cysteine of SEQ ID NO: 16 is not substituted with another amino acid. In one embodiment, said R4 in Formula (VI) or said R5 in Formula (VII) is an ankyrin repeat protein comprising an amino acid sequence that is 100% identical to SEQ ID NO: 16.

In another aspect, the invention relates to such a conjugate or pharmaceutically acceptable salt thereof, wherein said R4 in Formula (VI) or said R5 in Formula (VII) is an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 17. Thus, in one embodiment, said R4 in Formula (VI) or said R5 in Formula (VII) is an ankyrin repeat protein comprising an amino acid sequence that is at least 80% identical to SEQ ID NO: 17. In one embodiment, said R4 in Formula (VI) or said R5 in Formula (VII) is an ankyrin repeat protein comprising an amino acid sequence that is at least 85% identical to SEQ ID NO: 17. In one embodiment, said R4 in Formula (VI) or said R5 in Formula (VII) is an ankyrin repeat protein comprising an amino acid sequence that is at least 88% identical to SEQ ID NO: 17. In one embodiment, said R4 in Formula (VI) or said R5 in Formula (VII) is an ankyrin repeat protein comprising an amino acid sequence that is at least 90% identical to SEQ ID NO: 17. In one embodiment, said R4 in Formula (VI) or said R5 in Formula (VII) is an ankyrin repeat protein comprising an amino acid sequence that is at least 91% identical to SEQ ID NO: 17. In one embodiment, said R4 in Formula (VI) or said R5 in Formula (VII) is an ankyrin repeat protein comprising an amino acid sequence that is at least 92% identical to SEQ ID NO: 17. In one embodiment, said R4 in Formula (VI) or said R5 in Formula (VII) is an ankyrin repeat protein comprising an amino acid sequence that is at least 93% identical SEQ ID NO: 17. In one embodiment, said R4 in Formula (VI) or said R5 in Formula (VII) is an ankyrin repeat protein comprising an amino acid sequence that is at least 94% identical to SEQ ID NO: 17. In one embodiment, said R4 in Formula (VI) or said R5 in Formula (VII) is an ankyrin repeat protein comprising an amino acid sequence that is at least 95% identical to SEQ ID NO: 17. In one embodiment, said R4 in Formula (VI) or said R5 in Formula (VII) is an ankyrin repeat protein comprising an amino acid sequence that is at least 96% identical to SEQ ID NO: 17. In one embodiment, said R4 in Formula (VI) or said R5 in Formula (VII) is an ankyrin repeat protein comprising an amino acid sequence that is at least 97% identical to SEQ ID NO: 17. In one embodiment, said R4 in Formula (VI) or said R5 in Formula (VII) is an ankyrin repeat protein comprising an amino acid sequence that is at least 98% identical to SEQ ID NO: 17. In one embodiment, said R4 in Formula (VI) or said R5 in Formula (VII) is an ankyrin repeat protein comprising an amino acid sequence that is at least 99% identical to SEQ ID NO: 17. In one embodiment, any amino acid sequence differences between said amino acid sequence comprised in said ankyrin repeat protein (R4) in Formula (VI) or in said ankyrin repeat protein (R5) in Formula (VII) and SEQ ID NO: 17 represent amino acid substitutions in positions other than positions of potential target interaction residues of ankyrin repeat domains. In one embodiment, any amino acid sequence differences between said amino acid sequence comprised in said ankyrin repeat protein (R4) in Formula (VI) or in said ankyrin repeat protein (R5) in Formula (VII) and SEQ ID NO: 17 represent amino acid substitutions in (i) framework positions of ankyrin repeat domain(s), (ii) a peptide linker, (iii) a Glycine-Serine (GS), and/or (iv) a Cysteine-containing tag. In one embodiment, the C-terminal Cysteine of SEQ ID NO: 17 is not substituted with another amino acid. In one embodiment, said R4 in Formula (VI) or said R5 in Formula (VII) is an ankyrin repeat protein comprising an amino acid sequence that is 100% identical to SEQ ID NO: 17.

In another aspect, the invention relates to such a conjugate or pharmaceutically acceptable salt thereof, wherein said R4 in Formula (VI) or said R5 in Formula (VII) is an ankyrin repeat protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 18. Thus, in one embodiment, said R4 in Formula (VI) or said R5 in Formula (VII) is an ankyrin repeat protein comprising an amino acid sequence that is at least 80% identical to SEQ ID NO: 18. In one embodiment, said R4 in Formula (VI) or said R5 in Formula (VII) is an ankyrin repeat protein comprising an amino acid sequence that is at least 85% identical to SEQ ID NO: 18. In one embodiment, said R4 in Formula (VI) or said R5 in Formula (VII) is an ankyrin repeat protein comprising an amino acid sequence that is at least 88% identical to SEQ ID NO: 18. In one embodiment, said R4 in Formula (VI) or said R5 in Formula (VII) is an ankyrin repeat protein comprising an amino acid sequence that is at least 90% identical to SEQ ID NO: 18. In one embodiment, said R4 in Formula (VI) or said R5 in Formula (VII) is an ankyrin repeat protein comprising an amino acid sequence that is at least 91% identical to SEQ ID NO: 18. In one embodiment, said R4 in Formula (VI) or said R5 in Formula (VII) is an ankyrin repeat protein comprising an amino acid sequence that is at least 92% identical to SEQ ID NO: 18. In one embodiment, said R4 in Formula (VI) or said R5 in Formula (VII) is an ankyrin repeat protein comprising an amino acid sequence that is at least 93% identical SEQ ID NO: 18. In one embodiment, said R4 in Formula (VI) or said R5 in Formula (VII) is an ankyrin repeat protein comprising an amino acid sequence that is at least 94% identical to SEQ ID NO: 18. In one embodiment, said R4 in Formula (VI) or said R5 in Formula (VII) is an ankyrin repeat protein comprising an amino acid sequence that is at least 95% identical to SEQ ID NO: 18. In one embodiment, said R4 in Formula (VI) or said R5 in Formula (VII) is an ankyrin repeat protein comprising an amino acid sequence that is at least 96% identical to SEQ ID NO: 18. In one embodiment, said R4 in Formula (VI) or said R5 in Formula (VII) is an ankyrin repeat protein comprising an amino acid sequence that is at least 97% identical to SEQ ID NO: 18. In one embodiment, said R4 in Formula (VI) or said R5 in Formula (VII) is an ankyrin repeat protein comprising an amino acid sequence that is at least 98% identical to SEQ ID NO: 18. In one embodiment, said R4 in Formula (VI) or said R5 in Formula (VII) is an ankyrin repeat protein comprising an amino acid sequence that is at least 99% identical to SEQ ID NO: 18. In one embodiment, any amino acid sequence differences between said amino acid sequence comprised in said ankyrin repeat protein (R4) in Formula (VI) or in said ankyrin repeat protein (R5) in Formula (VII) and SEQ ID NO: 18 represent amino acid substitutions in positions other than positions of potential target interaction residues of ankyrin repeat domains. In one embodiment, any amino acid sequence differences between said amino acid sequence comprised in said ankyrin repeat protein (R4) in Formula (VI) or in said ankyrin repeat protein (R5) in Formula (VII) and SEQ ID NO: 18 represent amino acid substitutions in (i) framework positions of ankyrin repeat domain(s), (ii) a peptide linker, (iii) a Glycine-Serine (GS), and/or (iv) a Cysteine-containing tag. In one embodiment, the C-terminal Cysteine of SEQ ID NO: 18 is not substituted with another amino acid. In one embodiment, said R4 in Formula (VI) or said R5 in Formula (VII) is an ankyrin repeat protein comprising an amino acid sequence that is 100% identical to SEQ ID NO: 18.

In another aspect, the invention relates to a conjugate or pharmaceutically acceptable salt thereof, the conjugate comprising 1,4,7,10-Tetraazacyclododecane-1,4,7-tris(carbamoylmethyl)-10-maleimidoethylacetamide, an ankyrin repeat protein, and a radionuclide, wherein said ankyrin repeat protein comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 15 to 18, wherein said ankyrin repeat protein has binding specificity for DLL3 and for human serum albumin, wherein said ankyrin repeat protein comprises a Cysteine, wherein said ankyrin repeat protein is connected to said 1,4,7,10-Tetraazacyclododecane-1,4,7-tris(carbamoylmethyl)-10-maleimidoethylacetamide via a thioether bond formed between said Cysteine and the maleimide ring of said 1,4,7,10-Tetraazacyclododecane-1,4,7-tris(carbamoylmethyl)-10-maleimidoethylacetamide, wherein said radionuclide is Pb-212 or Pb-203, and wherein said radionuclide is coordinated by said 1,4,7,10-Tetraazacyclododecane-1,4,7-tris(carbamoylmethyl)-10-maleimidoethylacetamide. In one embodiment, said radionuclide is Pb-212. In one embodiment, said radionuclide is Pb-203. In one embodiment, said ankyrin repeat protein comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 15. In one embodiment, said ankyrin repeat protein comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 16. In one embodiment, said ankyrin repeat protein comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 17. In one embodiment, said ankyrin repeat protein comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 18.

In another aspect, the invention relates to such a conjugate or pharmaceutically acceptable salt thereof, wherein said ankyrin repeat protein binds human DLL3 (hDLL3) with a K_{D} value of or below 100 nM, of or below 30 nM, of or below 10 nM, of or below 3 nM, or of or below 1 nM, of or below 300 pM, of or below 100 pM, of or below 30 pM, or of or below 10 pM. Thus, in one embodiment, said ankyrin repeat protein binds hDLL3 with a K_{D} value of or below 100 nM. In one embodiment, said ankyrin repeat protein binds hDLL3 with a K_{D} value of or below 30 nM. In one embodiment, said ankyrin repeat protein binds hDLL3 with a K_{D} value of or below 10 nM. In one embodiment, said ankyrin repeat protein binds hDLL3 with a K_{D} value of or below 3 nM. In one embodiment, said ankyrin repeat protein binds hDLL3 with a K_{D} value or of or below 1 nM. In one embodiment, said ankyrin repeat protein binds hDLL3 with a K_{D} value of or below 400 pM. In one embodiment, said ankyrin repeat protein binds hDLL3 with a K_{D} value of or below 300 pM. In one embodiment, said ankyrin repeat protein binds hDLL3 with a K_{D} value of or below 200 pM. In one embodiment, said ankyrin repeat protein binds hDLL3 with a K_{D} value of or below 100 pM. In one embodiment, said ankyrin repeat protein binds hDLL3 with a K_{D} value of or below 30 pM. In one embodiment, said ankyrin repeat protein binds hDLL3 with a K_{D} value of or below 10 pM. Furthermore, in one embodiment, said ankyrin repeat protein binds to the extracellular domain of hDLL3. In one embodiment, said ankyrin repeat protein binds to the N-terminal domain of hDLL3.

In another aspect, the invention relates to such a conjugate or pharmaceutically acceptable salt thereof, wherein said ankyrin repeat protein binds human serum albumin with a K_{D} value of or below 500 nM, of or below 250 nM, or of or below 100 nM. In one embodiment, said ankyrin repeat protein binds human serum albumin with a K_{D} value of or below 500 nM. In one embodiment, said ankyrin repeat protein binds human serum albumin with a K_{D} value of or below 250 nM. In one embodiment, said ankyrin repeat protein binds human serum albumin with a K_{D} value of or below 100 nM.

In another aspect, the invention relates to any one of said conjugates or pharmaceutically acceptable salts thereof described herein in any of the disclosed aspects and embodiments, wherein said conjugate binds to cells expressing human DLL3 on their surface. In one embodiment, said cells are HEK293T-hDLL3 cells, wherein HEK293T-hDLL3 cells are HEK293T cells engineered to express human DLL3 on their surface. In one embodiment, said conjugate binds human DLL3-expressing HEK293T-hDLL3 cells with an EC50 below 10⁻⁷M, or about or below 5×10⁻⁸M, or about or below 2×10⁻⁸M, or about or below 10⁻⁸M, or about or below 5×10⁻⁹M, or about or below 10⁻⁹M. Thus, in one embodiment, said conjugate binds human DLL3-expressing HEK293T-hDLL3 cells with an EC50 below 10⁻⁷M, and in another embodiment, said conjugate binds human DLL3-expressing HEK293T-hDLL3 cells with an EC50 about or below 5×10⁻⁸M. In one embodiment, said conjugate binds human DLL3-expressing HEK293T-hDLL3 cells with an EC50 about or below 2×10⁻⁸M. In one embodiment, said conjugate binds human DLL3-expressing HEK293T-hDLL3 cells with an EC50 about or below 10⁻⁸M. In one embodiment, said conjugate binds human DLL3-expressing HEK293T-hDLL3 cells with an EC50 about or below 5×10⁻⁹M, and in another embodiment, said conjugate binds human DLL3-expressing HEK293T-hDLL3 cells with an EC50 or about or below 10⁻⁹M.

In another aspect, the invention relates to any one of said conjugates or pharmaceutically acceptable salts thereof described herein in any of the disclosed aspects and embodiments, wherein said conjugate binds to cells expressing human DLL3 on their surface, and wherein said cells are NCI-H82 cells. NCI-H82 lung carcinoma cells naturally (endogenously) express human DLL3 on their cell surface. In one embodiment, said conjugate binds NCI-H82 cells with an EC50 below 10⁻⁷M, or about or below 3×10⁻⁸M, or about or below 10⁻⁸M, or about or below 6×10⁻⁹M, or about or below 3×10⁻⁹M, or about or below 10⁻⁹M. Thus, in one embodiment, said conjugate binds NCI-H82 cells with an EC50 below 10⁻⁷M, and in another embodiment, said conjugate binds NCI-H82 cells with an EC50 about or below 3×10⁻⁸M. In one embodiment, said conjugate binds NCI-H82 cells with an EC50 about or below 10⁻⁸M. In one embodiment, said conjugate binds NCI-H82 cells with an EC50 about or below 6×10⁻⁹M. In one embodiment, said conjugate binds NCI-H82 cells with an EC50 about or below 3×10⁻⁹M, and in another embodiment, said conjugate binds NCI-H82 cells with an EC50 or about or below 10⁻⁹M.

In another aspect, the invention relates to any one of said conjugates or pharmaceutically acceptable salts thereof described herein in any of the disclosed aspects and embodiments, wherein said conjugate has a terminal half-life in a mouse model of at least about 5 hours, at least about 8 hours, at least about 10 hours, at least about 12 hours, at least about 15 hours, at least about 18 hours, at least about 20 hours, at least about 23 hours, at least about 25 hours, at least about 28 hours, at least about 30 hours, at least about 33 hours, at least about 35 hours, or at least about 40 hours. In one embodiment, said conjugate has a terminal half-life in a mouse model of about 5 to 45 hours, about 5 to 40 hours, about 10 to 40 hours, about 20 to 40 hours, about 25 to 40 hours, about 30 to 40 hours, about 5 to 35 hours, about 10 to 35 hours, about 20 to 35 hours, about 25 to 35 hours, or about 30 to 35 hours. In one embodiment, said conjugate has a terminal half-life in a mouse model of at least about 25 hours. In another embodiment, said conjugate has a terminal half-life in a mouse model of at least about 30 hours. In one embodiment, said conjugate has a terminal half-life in a mouse model of about 25 to 40 hours. In another embodiment, said conjugate has a terminal half-life in a mouse model of about 30 to 40 hours. In another embodiment, said conjugate has a terminal half-life in a mouse model of about 25 to 35 hours. In another embodiment, said conjugate has a terminal half-life in a mouse model of about 30 to 35 hours. In one embodiment, said terminal half-life of said conjugate is measured in a BALB/c mouse model. In one embodiment, said terminal half-life of said conjugate is measured after intravenous injection of 1 mg/kg of conjugate into said mouse model.

In another aspect, the invention relates to any one of said conjugates or pharmaceutically acceptable salts thereof described herein in any of the disclosed aspects and embodiments, wherein said radionuclide is Pb-212, and wherein said conjugate is capable of inhibiting tumor growth in a human DLL3-expressing mouse tumor model. In one embodiment, said human DLL3-expressing mouse tumor model is a hDLL3-expressing MC38 (hDLL3-MC38) colon carcinoma mouse model or a NCI-H82 lung carcinoma mouse model. In one embodiment, said human DLL3-expressing mouse tumor model is a hDLL3-expressing MC38 (hDLL3-MC38) colon carcinoma mouse model. To generate the hDLL3-MC38 mouse tumor model, MC38 cells were engineered to express human DLL3 on their surface. In one embodiment, said conjugate is capable of inhibiting tumor growth in a hDLL3-expressing MC38 (hDLL3-MC38) colon carcinoma mouse model, wherein conditions for tumor growth and treatment are as described in Example 5. In another embodiment, said human DLL3-expressing mouse tumor model is a NCI-H82 lung carcinoma mouse model. NCI-H82 lung carcinoma cells naturally (endogenously) express human DLL3 on their cell surface. In one embodiment, said conjugate is capable of inhibiting tumor growth in a NCI-H82 lung carcinoma mouse model, wherein conditions for tumor growth and treatment are as described in Example 16.

In another aspect, the invention relates to any one of said conjugates or pharmaceutically acceptable salts thereof described herein in any of the disclosed aspects and embodiments, wherein said conjugate reaches a tumor-to-kidney ratio (T:K) in a hDLL3-expressing mouse tumor model at 24 hours after administration of said conjugate of at least about 1.0, at least about 1.2, at least about 1.5, at least about 1.7, at least about 2.0, at least about 2.2, or at least about 2.5. Thus, in one embodiment, said conjugate reaches a tumor-to-kidney ratio (T:K) of at least about 1.0. In one embodiment, said conjugate reaches a tumor-to-kidney ratio (T:K) of at least about 1.2. In one embodiment, said conjugate reaches a tumor-to-kidney ratio (T:K) of at least about 1.5. In one embodiment, said conjugate reaches a tumor-to-kidney ratio (T:K) of at least about 1.7. In one embodiment, said conjugate reaches a tumor-to-kidney ratio (T:K) of at least about 2.0. In one embodiment, said conjugate reaches a tumor-to-kidney ratio (T:K) of at least about 2.2. In one embodiment, said conjugate reaches a tumor-to-kidney ratio (T:K) of at least about 2.5. In one embodiment, said hDLL3-expressing mouse tumor model is a hDLL3-expressing MC38 colon carcinoma mouse model or a NCI-H82 lung carcinoma mouse model. In one embodiment, said hDLL3-expressing mouse tumor model is a hDLL3-expressing MC38 colon carcinoma mouse model. In one embodiment, said hDLL3-expressing mouse tumor model is a NCI-H82 lung carcinoma mouse model.

### Pharmaceutical Compositions and Kits

In another aspect, the invention relates to a pharmaceutical composition comprising any one of said conjugates or pharmaceutically acceptable salts thereof described herein in any of the aforementioned aspects and embodiments, and optionally a pharmaceutically acceptable carrier, excipient, stabilizer and/or diluent. In one exemplary embodiment, the invention relates to a pharmaceutical composition comprising a conjugate or pharmaceutically acceptable salt thereof, and optionally a pharmaceutically acceptable carrier, excipient, stabilizer and/or diluent, wherein said conjugate comprises (i) an ankyrin repeat domain with binding specificity for DLL3, (ii) a chelator, and (iii) a radionuclide, wherein said chelator is covalently connected to said ankyrin repeat domain with binding specificity for DLL3, wherein said radionuclide is bound to said chelator, wherein said radionuclide is Pb-212, and wherein said ankyrin repeat domain with binding specificity for DLL3 comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 1 to 4. In another exemplary embodiment, the invention relates to a pharmaceutical composition comprising a conjugate or pharmaceutically acceptable salt thereof, and optionally a pharmaceutically acceptable carrier, excipient, stabilizer and/or diluent, wherein said conjugate comprises (i) an ankyrin repeat domain with binding specificity for DLL3, (ii) a connector, (iii) a chelator, and (iv) a radionuclide, wherein said connector is covalently connected to said ankyrin repeat domain with binding specificity for DLL3, wherein said chelator is covalently connected to said connector, wherein said radionuclide is bound to said chelator, wherein said radionuclide is Pb-212, and wherein said ankyrin repeat domain with binding specificity for DLL3 comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 1 to 4.

Pharmaceutically acceptable carriers, excipients, stabilizers and/or diluents are known to the person skilled in the art and are explained in more detail below.

In one embodiment, a pharmaceutical composition comprises a conjugate or pharmaceutically acceptable salt thereof according to the present invention, and a pharmaceutically acceptable carrier, excipient, stabilizer and/or diluent, for example as described in Remington, The Science and Practice of Pharmacy; 23rd edition; Adeboye A. Ed., 2020.

Pharmaceutically acceptable carriers, excipients, stabilizers and/or diluents known to one of skill in the art include, for example, saline, Ringer's solution, dextrose solution, Hank's solution, fixed oils, ethyl oleate, 5% dextrose in saline, substances that enhance isotonicity and chemical stability, buffers and preservatives. Other potentially suitable carriers include any carrier that does not itself induce the production of antibodies harmful to the individual receiving the composition such as proteins, polysaccharides, polylactic acids, polyglycolic acids, polymeric amino acids and amino acid copolymers. A pharmaceutical composition may also comprise an antioxidant and/or a scavenger. A pharmaceutical composition may also be a combination formulation, comprising an additional active agent, such as an anti-cancer agent or an anti-angiogenic agent, or an additional bioactive compound.

In one embodiment, a pharmaceutical composition comprises a conjugate or pharmaceutically acceptable salt thereof according to the present invention, and a detergent such as nonionic detergent, a buffer such as phosphate buffer, and/or a sugar such as sucrose. In one embodiment, a pharmaceutical composition comprises a conjugate or pharmaceutically acceptable salt thereof according to the present invention, and PBS.

The formulations to be used for in vivo administration must be aseptic or sterile. This can be readily accomplished, e.g., by filtration through sterile filtration membranes.

In one embodiment, the present invention relates to the use of a conjugate or pharmaceutically acceptable salt thereof, as described herein, for manufacturing a pharmaceutical composition. In one exemplary embodiment, the present invention relates to the use of a conjugate or pharmaceutically acceptable salt thereof, as described herein, for manufacturing a pharmaceutical composition, wherein said conjugate comprises (i) an ankyrin repeat domain with binding specificity for DLL3, (ii) a chelator, and (iii) a radionuclide, wherein said chelator is covalently connected to said ankyrin repeat domain with binding specificity for DLL3, wherein said radionuclide is bound to said chelator, wherein said radionuclide is Pb-212, and wherein said ankyrin repeat domain with binding specificity for DLL3 comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 1 to 4. In another exemplary embodiment, the present invention relates to the use of a conjugate or pharmaceutically acceptable salt thereof, as described herein, for manufacturing a pharmaceutical composition, wherein said conjugate comprises (i) an ankyrin repeat domain with binding specificity for DLL3, (ii) a connector, (iii) a chelator, and (iv) a radionuclide, wherein said connector is covalently connected to said ankyrin repeat domain with binding specificity for DLL3, wherein said chelator is covalently connected to said connector, wherein said radionuclide is bound to said chelator, wherein said radionuclide is Pb-212, and wherein said ankyrin repeat domain with binding specificity for DLL3 comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 1 to 4.

In another aspect, the invention relates to a kit comprising (i) a first container containing any one of said conjugates or pharmaceutically acceptable salts thereof or said pharmaceutical compositions described herein in any of the aforementioned aspects and embodiments; and (ii) a second container containing a buffered solution.

Methods of Imaging, Diagnosing and/or Treating Medical Conditions

In another aspect, the invention relates to any one of said conjugates or pharmaceutically acceptable salts thereof or said pharmaceutical composition, described herein in any of the aforementioned aspects and embodiments, for use in imaging, diagnosing and/or treating a medical condition. In one aspect, the invention relates to any one of said conjugates or pharmaceutically acceptable salts thereof or said pharmaceutical composition, described herein in any of the aforementioned aspects and embodiments, for use in a method of imaging, diagnosing and/or treating a medical condition, the method comprising the step of administering to a subject in need thereof an amount of said conjugate or salt or of said pharmaceutical composition effective for imaging, diagnosing and/or treating said medical condition.

In a more particular aspect, the invention relates to any one of said conjugates or pharmaceutically acceptable salts thereof or said pharmaceutical composition, described herein in any of the aforementioned aspects and embodiments, for use in a method of treating a medical condition, the method comprising the step of administering to a subject in need thereof an amount of said conjugate or salt or of said pharmaceutical composition effective for treating said medical condition. In one embodiment, said radionuclide comprised in said conjugates or pharmaceutically acceptable salts thereof or in said pharmaceutical compositions for use in a method of treating a medical condition is Pb-212.

In another more particular aspect, the invention relates to any one of said conjugates or pharmaceutically acceptable salts thereof or said pharmaceutical compositions, described herein in any of the aforementioned aspects and embodiments, for use in a method of imaging and/or diagnosing a medical condition, the method comprising the step of administering to a subject an amount of said conjugate or salt or of said pharmaceutical composition effective for imaging and/or diagnosing said medical condition. In one embodiment, said radionuclide comprised in said conjugates or pharmaceutically acceptable salts thereof or in said pharmaceutical compositions for use in a method of imaging and/or diagnosing a medical condition is Pb-203.

In another aspect, the invention relates to a method of treating a medical condition, the method comprising the step of administering to a subject in need thereof a therapeutically effective amount of any one of said conjugates or pharmaceutically acceptable salts thereof or said pharmaceutical compositions described herein in any of the aforementioned aspects and embodiments. In one embodiment, said radionuclide comprised in said conjugate or pharmaceutically acceptable salt thereof or in said pharmaceutical composition administered to said subject is Pb-212.

In another aspect, the invention relates to a method of imaging and/or diagnosing a medical condition, the method comprising the step of administering to a subject an amount of any one of said conjugates or pharmaceutically acceptable salts thereof or said pharmaceutical compositions described herein in any of the aforementioned aspects and embodiments, effective for imaging and/or diagnosing said medical condition. In one embodiment, said method of imaging and/or diagnosing a medical condition comprises the steps of (i) administering to a subject an amount of any one of said conjugates or pharmaceutically acceptable salts thereof or said pharmaceutical compositions, effective for binding of conjugate or salt thereof to cells expressing DLL3 on their surface, and (ii) detecting cells bound by conjugate or salt thereof and/or tissues comprising cells bound by conjugate or salt thereof. In one embodiment, said detecting in step (ii) is performed by in vivo imaging. In one embodiment, said detecting in step (ii) is performed by in vivo imaging, wherein said in vivo imaging uses single photon emission computed tomography (SPECT). In one embodiment, said radionuclide comprised in said conjugate or pharmaceutically acceptable salt thereof or in said pharmaceutical composition administered to said subject is Pb-203.

In one embodiment, in any of said conjugates or pharmaceutically acceptable salts thereof or said pharmaceutical compositions for use in a method of imaging, diagnosing and/or treating a medical condition, or in any of said methods of treating a medical condition or of imaging and/or diagnosing a medical condition, said subject is a mammal, preferably a human. In one embodiment, said medical condition is cancer. In one embodiment, said cancer comprises cells that express DLL3 on their surface. In one embodiment, said cancer is a neuroendocrine cancer. In one embodiment, said cancer is glioma, neuroendocrine lung cancer, neuroendocrine prostate cancer, gastrointestinal neuroendocrine cancer or small cell bladder cancer. In one embodiment, said cancer is small cell lung cancer. In one embodiment, said cancer is small cell lung carcinoma.

For use of a conjugate or pharmaceutically acceptable salt thereof or of a pharmaceutical composition in a method of imaging, diagnosing and/or treating a medical condition, said conjugates or pharmaceutically acceptable salts thereof or said pharmaceutical compositions according to the present invention are typically administered to a subject by parenteral administration. For parental administration, said conjugates or pharmaceutically acceptable salts thereof or said pharmaceutical compositions according to the present invention will typically be formulated in a unit dosage injectable form such as a solution, suspension or emulsion, in association with pharmaceutically acceptable carriers, excipients, stabilizers and/or diluents as defined above. The dosage and mode of administration will depend on the individual to be imaged, diagnosed and/or treated, the particular medical condition, and the purpose of the administration. Parenteral administration may occur, for example, by injection. Parenteral injections may be done via different routes, such as, e.g., intradermal (IM), subcutaneous (SQ), intramuscular (IM), and intravenous (IV) injections. Furthermore, parenteral injections may be done as bolus injection or by slow infusion.

Any of the above-mentioned conjugates or pharmaceutically acceptable salts thereof or pharmaceutical compositions according to the present invention are considered for use in the imaging, diagnosing and/or treatment of a disorder, disease or medical condition. The terms disorder, disease and medical condition are used interchangeably herein.

In one embodiment, any of said conjugates or pharmaceutically acceptable salts thereof or pharmaceutical compositions according to the present invention is administered intravenously or subcutaneously. In one embodiment, any of said conjugates or pharmaceutically acceptable salts thereof or pharmaceutical compositions according to the present invention is administered intravenously. In one embodiment, any of said conjugates or pharmaceutically acceptable salts thereof or pharmaceutical compositions according to the present invention is administered subcutaneously.

The use of a conjugate or pharmaceutically acceptable salt thereof or of a pharmaceutical composition according to the present invention for the treatment of a medical condition, such as cancer, can also be in combination with one or more other therapies known in the art. The term “use in combination with”, as used herein, shall refer to a co-administration, which is carried out under a given regimen. This includes synchronous administration of different compounds as well as time-shifted administration of different compounds (e.g. compound A is given once and compound B is given several times thereafter, or vice versa, or both compounds are given synchronously and one of the two is also given at later stages).

In another aspect, the invention relates to any one of said conjugates or pharmaceutically acceptable salts thereof or said pharmaceutical compositions according to the present invention, for use in a process of manufacturing a medicament. In one embodiment, said medicament is for the treatment of a medical condition, e.g. cancer. In one embodiment, said medicament is for the treatment of a neuroendocrine cancer, e.g. small cell lung cancer.

In another aspect, the invention relates to a process of manufacturing a medicament for the treatment of a medical condition, wherein a conjugate or pharmaceutically acceptable salt thereof or a pharmaceutical composition according to the present invention is an active ingredient of said medicament. In one embodiment, said medical condition is cancer, e.g. a neuroendocrine cancer. In one embodiment, said medical condition is a neuroendocrine lung cancer, such as small cell lung cancer.

The above Detailed Description of the Invention provides numerous aspects and embodiments, describing different features of the disclosed subject matter. It should be understood that certain features described in these aspects and embodiments may be combined, as indicated for example in the embodiments E1 to E95 provided in the Summary of the Invention section.

### Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, chemistry, immunology, microbiology, genetics and protein and nucleic acid chemistry described herein are those well-known and commonly used in the art.

The terms “comprising”, “having”, “including” and “containing” are to be construed as open-ended terms unless otherwise noted. If aspects of the invention are described as “comprising” a feature, embodiments also are contemplated “consisting of” or “consisting essentially of” the feature. The use of any and all examples, or exemplary language (e.g., “such as”) provided herein, is intended merely to better illustrate the disclosure and does not pose a limitation on the scope of the disclosure unless otherwise stated. No language in the specification should be construed as indicating any non-claimed element as essential to the practice of the disclosure. The term “about” as used herein is equivalent to ±10% of a given numerical value, unless otherwise stated.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

The term “nucleic acid” refers to a polynucleotide molecule, which may be a ribonucleic acid (RNA) or deoxyribonucleic acid (DNA) molecule, either single stranded or double stranded, and includes modified and artificial forms of DNA or RNA. A nucleic acid may either be present in isolated form or be comprised in recombinant nucleic acid molecules or vectors.

In the context of the present invention the term “protein” refers to a molecule comprising a polypeptide, wherein at least part of the polypeptide has, or is able to acquire, a defined three-dimensional arrangement by forming secondary, tertiary, and/or quaternary structures within a single polypeptide chain and/or between multiple polypeptide chains. If a protein comprises two or more polypeptide chains, the individual polypeptide chains may be linked non-covalently or covalently, e.g. by a disulfide bond between two polypeptides. A part of a protein, which individually has, or is able to acquire, a defined three-dimensional arrangement by forming secondary and/or tertiary structure, is termed “protein domain”. Such protein domains are well known to the practitioner skilled in the art.

The term “recombinant” as used in recombinant protein, recombinant polypeptide and the like, means that said protein or polypeptide is produced by the use of recombinant DNA technologies well known to the practitioner skilled in the art. For example, a recombinant DNA molecule (e.g. produced by gene synthesis) encoding a polypeptide can be cloned into a bacterial expression plasmid (e.g. pQE30, QIAgen), yeast expression plasmid, mammalian expression plasmid, or plant expression plasmid, or a DNA enabling in vitro expression. If, for example, such a recombinant bacterial expression plasmid is inserted into appropriate bacteria (e.g. Escherichia coli), these bacteria can produce the polypeptide(s) encoded by this recombinant DNA. The correspondingly produced polypeptide or protein is called a recombinant polypeptide or recombinant protein.

In the context of the present invention, the term “polypeptide” relates to a molecule consisting of a chain of multiple, i.e. two or more, amino acids linked via peptide bonds. Preferably, a polypeptide consists of more than eight amino acids linked via peptide bonds. The term “polypeptide” also includes multiple chains of amino acids, linked together by S—S bridges of cysteines. Polypeptides are well-known to the person skilled in the art.

The term “target” refers to an individual molecule such as a nucleic acid, a polypeptide or protein, a carbohydrate, or any other naturally or non-naturally occurring molecule or moiety, including any part of such individual molecule, or complexes of two or more of such molecules. The target may be a whole cell or a tissue sample. Preferably, the target is a naturally occurring or non-natural polypeptide or a polypeptide containing chemical modifications, for example modified by natural or non-natural phosphorylation, acetylation, or methylation.

Patent application WO2002020565 and Forrer et al., 2003 (Forrer, P., Stumpp, M. T., Binz, H. K., Plückthun, A., 2003. FEBS Letters 539, 2-6), contain a general description of repeat protein features and repeat domain features, techniques and applications. The term “repeat protein” refers to a protein comprising one or more repeat domains. Preferably, a repeat protein comprises one, two, three, four, five or six repeat domains. Furthermore, said repeat protein may comprise additional non-repeat protein domains, polypeptide tags and/or peptide linkers.

The term “repeat domain” refers to a protein domain comprising two or more consecutive repeat modules as structural units, wherein said repeat modules have structural and sequence homology. Preferably, a repeat domain also comprises an N-terminal and/or a C-terminal capping module. For clarity, a capping module can be a repeat module. Such repeat domains, repeat modules, and capping modules, sequence motifs, as well as structural homology and sequence homology are well known to the practitioner in the art from examples of ankyrin repeat domains (Binz et al., J. Mol. Biol. 332, 489-503, 2003; Binz et al., 2004, loc. cit.; WO2002020565; WO2012069655), leucine-rich repeat domains (WO2002020565), tetratricopeptide repeat domains (Main, E. R., Xiong, Y., Cocco, M. J., D'Andrea, L., Regan, L., Structure 11(5), 497-508, 2003), and armadillo repeat domains (WO2009040338). It is further well known to the practitioner in the art that such repeat domains are different from proteins comprising repeated amino acid sequences, where every repeated amino acid sequence is able to form an individual domain (for example FN3 domains of Fibronectin). The repeat domains can be binding domains.

The term “ankyrin repeat domain” refers to a repeat domain comprising two or more consecutive ankyrin repeat modules as structural units, wherein said ankyrin repeat modules have structural and sequence homology.

The term “designed” as used in designed repeat protein, designed repeat domain and the like refers to the property that such repeat proteins and repeat domains, respectively, are man-made and do not occur in nature. The binding domains of the instant invention are designed repeat domains. Preferably, a designed repeat domain of the invention is a designed ankyrin repeat domain.

A residue or amino acid residue refers to an amino acid comprised in a peptide chain. The term “target interaction residues” refers to amino acid residues of a repeat module, which contribute to the direct interaction with a target. Such contribution of a residue can be tested, e.g., in a binding assay, for example in a mutagenesis study performed to identify residues required, sufficient, and/or necessary for a repeat domain to bind a target with its original binding affinity or quantity (i.e. its binding affinity or quantity in the absence of any mutations). Target interaction residues can also be determined by structural analyses of a repeat domain bound to a target.

The term “framework residues” refers to amino acid residues of a repeat module, which contribute to the folding topology, i.e. which contribute to the fold of said repeat module or which contribute to the interaction with a neighboring module. Such contribution may be the interaction with other residues in the repeat module, or the influence on the polypeptide backbone conformation as found in α-helices or β-sheets, or the participation in amino acid stretches forming linear polypeptides or loops.

Such framework and target interaction residues may be identified by analysis of the structural data obtained by physicochemical methods, such as X-ray crystallography, NMR and/or CD spectroscopy, or by comparison with known and related structural information well known to practitioners in structural biology and/or bioinformatics.

Preferably, framework residues shall correspond to residues occupying specific positions within repeat modules as described in Table A:

TABLE A

| Repeat | | Position of framework |
|---|---|---|
| modules | Reference sequence | residues |
| N-terminal | SEQ ID NO: 23 | 1 to 3, 5 to 7, |
| capping module | | 9, 10, 13 to 30 |
| Internal repeat | SEQ ID NO: 24 | 1, 2, 5, 7 to 10, |
| module | | 12, 13, 16 to 33 |
| C-terminal | SEQ ID NO: 25 | 1, 2, 5, 7 to 13, 16 to 28 |
| capping module | | |

Table B shows preferred positions of potential target interaction residues in designed ankyrin repeat domains with binding specificity for a target.

TABLE B

| Repeat | Reference | Position of potential |
|---|---|---|
| modules | sequence | target interaction residues |
| N-terminal | SEQ ID NO: 23 | 4, 8, 11 and 12 |
| capping module | | |
| Internal | SEQ ID NO: 24 | 3, 4, 6, 11, 14 and 15 |
| repeat module | | |
| C-terminal | SEQ ID NO: 25 | 3, 4, 6, 14 and 15 |
| capping module | | |

Specifically for the purpose of defining positions in amino acid sequences of ankyrin repeat domains and proteins provided herein (including SEQ ID NOs: 1 to 7 and 11 to 18), at which amino acid substitutions are permitted or are not permitted in some embodiments of the invention described herein, the term “framework residues” includes the amino acid residues located at the positions within a designed ankyrin repeat domain that correspond to the positions listed in Table A for the representative N-terminal capping module (i.e. positions 1 to 3, 5 to 7, 9, 10, and 13 to 30 of SEQ ID NO: 23), the representative internal repeat module (i.e. positions 1, 2, 5, 7 to 10, 12, 13, and 16 to 33 of SEQ ID NO: 24) and the representative C-terminal capping module (i.e. positions 1, 2, 5, 7 to 13, and 16 to 28 of SEQ ID NO: 25). The term “framework residues” does not include the amino acid residues located at the positions within a designed ankyrin repeat domain that correspond to the positions listed in Table B for the representative N-terminal capping module (i.e. positions 4, 8, 11 and 12 of SEQ ID NO: 23), the representative internal repeat module (i.e. positions 3, 4, 6, 11, 14 and 15 of SEQ ID NO: 24) and the representative C-terminal capping module (i.e. positions 3, 4, 6, 14 and 15 of SEQ ID NO: 25).

Specifically for the purpose of defining positions in amino acid sequences of ankyrin repeat domains and proteins provided herein (including SEQ ID NOs: 1 to 7 and 11 to 18), at which amino acid substitutions are permitted or are not permitted in some embodiments of the invention described herein, the term “potential target interaction residues” includes the amino acid residues located at the positions within a designed ankyrin repeat domain that correspond to the positions listed in Table B for the representative N-terminal capping module (i.e. positions 4, 8, 11 and 12 of SEQ ID NO: 23), the representative internal repeat module (i.e. positions 3, 4, 6, 11, 14 and 15 of SEQ ID NO: 24) and the representative C-terminal capping module (i.e. positions 3, 4, 6, 14 and 15 of SEQ ID NO: 25). The term “potential target interaction residues” does not include the amino acid residues located at the positions within a designed ankyrin repeat domain that correspond to the positions listed in Table A for the representative N-terminal capping module (i.e. positions 1 to 3, 5 to 7, 9, 10, and 13 to 30 of SEQ ID NO: 23), the representative internal repeat module (i.e. positions 1, 2, 5, 7 to 10, 12, 13, and 16 to 33 of SEQ ID NO: 24) and the representative C-terminal capping module (i.e. positions 1, 2, 5, 7 to 13, and 16 to 28 of SEQ ID NO: 25).

In some embodiments, an amino acid substitution in a sequence provided herein (such as, e.g., SEQ ID NOs: 1 to 7 and 11 to 18) is an exemplary substitution according to Table C. In some embodiments, an amino acid substitution in a sequence provided herein (such as, e.g., SEQ ID NOs: 1 to 7 and 11 to 18) is a conservative substitution according to Table C. In some embodiments, the substitution is made outside the structural core residues of an ankyrin repeat domain, e.g., in the beta loops that connect the alpha-helices.

TABLE C
Amino acid substitutions

| Original | Conservative | |
|---|---|---|
| Residue | Substitutions | Exemplary Substitutions |
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; Norleucine |
| Leu (L) | Ile | Norleucine; Ile; Val; Met; Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr(Y) | Phe | Trp; Phe; Thr; Ser |
| Val (V) | Leu | Ile; Leu; Met; Phe; Ala; Norleucine |

The term “binding specificity”, “has binding specificity for a target”, “specifically binding to a target”, “binding to a target with high specificity”, “specific for a target” or “target specificity” and the like means that a binding protein or binding domain binds to a target with a lower dissociation constant (i.e. it binds with higher affinity) than it binds to an unrelated protein such as the E. coli maltose binding protein (MBP). Preferably, the dissociation constant (“Ko”) for the target is at least 10²; more preferably, at least 10³; more preferably, at least 10⁴; or more preferably, at least 10⁵ times lower than the corresponding dissociation constant for MBP. Methods to determine dissociation constants of protein-protein interactions, such as surface plasmon resonance (SPR) based technologies (e.g. SPR equilibrium analysis) or isothermal titration calorimetry (ITC) are well known to the person skilled in the art. The measured K_{D} values of a particular protein-protein interaction can vary if measured under different conditions (e.g., salt concentration, pH). Thus, measurements of K_{D} values are preferably made with standardized solutions of protein and a standardized buffer, such as PBS.

Binding of any molecule to another is governed by two forces, namely the association rate (kₒₙ) and the dissociation rate (k_{off}). The affinity of any binder [B] to a target [T] can then be expressed by the equilibrium dissociation constant K_{D}, which is the quotient of k_{off}/kₒₙ.

$$[ B ] + [ T ] {⇌}_{{k}_{off}}^{{k}_{on}} [ BT ]$$

kₒₙ is a second-order rate constant of the binding reaction, with the unit M⁻¹s⁻¹, whereas the dissociation reaction k_{off} is a first-order rate constant with the unit s⁻¹. From this it becomes clear that the association reaction depends on the concentration of the reactants, whereas the dissociation is independent of the concentration, following a simple exponential decay function.

A variety of methods of measuring binding affinity are known in the art, any of which can be used for purposes of the present invention. For example, as exemplified herein, the binding affinity of a particular binding moiety to a drug molecule target can be expressed as K_{D} value, which refers to the dissociation constant of the binding moiety and the drug molecule target. K_{D} is the ratio of the rate of dissociation, also called the “off-rate (k_{off})”, to the association rate, or “on-rate (kₒₙ)”. Thus, K_{D} equals k_{off}/kₒₙ and is expressed as a molar concentration (M), and the smaller the K_{D}, the stronger the affinity of binding.

K_{D} values can be determined using any suitable method. One exemplary method for measuring K_{D} is surface plasmon resonance (SPR) (see, e.g., Nguyen et al. Sensors (Basel). 2015 May 5; 15(5):10481-510). K_{D} value may be measured by SPR using a biosensor system such as a BIACORE® system. BIAcore kinetic analysis comprises, e.g., analysing the binding and dissociation of an antigen from chips with immobilized molecules (e.g., molecules comprising epitope binding domains), on their surface. Another method for determining the K_{D} of a protein is by using Bio-Layer Interferometry (see, e.g., Shah et al. J Vis Exp. 2014; (84): 51383). A K_{D} value may be measured using OCTET® technology (Octet Qke system, ForteBio). Alternatively, or in addition, a KinExA® (Kinetic Exclusion Assay) assay, available from Sapidyne Instruments (Boise, Id.) can also be used. Any method suitable for assessing the binding affinity between two binding partners is encompassed herein. Surface plasmon resonance (SPR) is particularly preferred. Most preferably, the K_{D} values are determined in PBS and by SPR.

The term “PBS” means a phosphate buffered water solution containing 137 mM NaCl, 10 mM phosphate and 2.7 mM KCl and having a pH of 7.4.

The term “mouse serum albumin” refers to UniProt accession number P07724, the term “cynomolgus monkey serum albumin” (i.e. Macaca fascicularis) refers to UniProt accession number A2V9Z4, and the term “human serum albumin” refers to UniProt accession number P02768 and SEQ ID NO: 28. The amino acid sequence of the isoform of human serum albumin that has been chosen as the canonical sequence is provided in SEQ ID NO: 28. The sequence is 609 amino acids long. Residues 25 to 609 of SEQ ID NO: 28 form the human serum albumin chain.

The human DLL3 protein is described and its amino acid sequence is provided in UniProt accession number Q9NYJ7. The amino acid sequence of the isoform of human DLL3 that has been chosen as the canonical sequence is provided in SEQ ID NO: 27. The sequence is 618 amino acids long. Residues 27 to 492 of SEQ ID NO: 27 form the extracellular domain of human DLL3, residues 493 to 513 of SEQ ID NO: 27 form the transmembrane domain of human DLL3, and residues 514 to 618 of SEQ ID NO: 27 form the cytoplasmic domain of human DLL3.

The DLL3 protein of cynomolgus monkey (i.e. cyno DLL3) is described and its amino acid sequence provided in UniProt accession number A0A2K5WSR1. The mouse DLL3 protein is described and its amino acid sequence provided in UniProt accession number 088516.

The term pharmacokinetic properties refers to various pharmacokinetic parameters, including area under the curve, clearance, and terminal half-life (or serum half-life). These parameters of pharmacokinetic properties and ways to determine them are well known in the art (see, e.g., Mahmood, I., Methods to determine pharmacokinetic profiles of therapeutic proteins, Drug Discov Today: Technol (2009), doi:10.1016/j.ddtec.2008.12.001).

The term “linked” or “linkage” refers to any covalent or non-covalent linkage between two chemical and/or biochemical moieties, e.g. between a chemical moiety and a protein such as a designed repeat domain or a designed repeat protein. The term “connected”, “covalently connected” or “covalent connection” refers to any covalent linkage between two chemical and/or biochemical moieties. Such moieties include, for example, a connector, a chelator, and a polypeptide such as a designed repeat domain or a designed repeat protein.

The term “radionuclide” or “radioisotope” refers to isotopes of natural or artificial origin with an unstable neutron to proton ratio that disintegrates with the emission of corpuscular (i.e. protons (alpha-radiation) or electrons (beta-radiation)) or electromagnetic radiation (gamma-radiation). In other words, radionuclides undergo radioactive decay. Such radionuclides include, without limitation, ⁹⁴Tc, ^{99m}Tc, ⁹⁰In, ¹¹¹In, ⁶⁷Ga, ⁶⁸Ga, ⁸⁶Y, ⁹⁰Y, ¹⁷⁷Lu, ¹⁵¹Tb, ²²³Ra, ¹⁸⁶Re, ¹⁸⁸Re, ⁶⁴Cu, ⁶⁷Cu, ⁵⁵Co, ⁵⁷Co, ⁴³Sc, ⁴⁴Sc, ⁴⁷Sc, ²³⁵Ac, ²¹³Bi, ²¹²Bi, ²⁰³Pb, ²¹²Pb, ²²⁷Th, ¹⁵³Sm, ¹⁶⁶Ho, ¹⁵²Gd, ¹⁵³Gd, ¹⁵⁷Gd, ²²⁵Ac or ¹⁶⁶Dy. The choice of suitable radionuclides may depend on the chemical structure and chelating capability of the chelating agent (or chelator), and the intended application of the resulting compound (e.g. diagnostic, therapeutic or imaging). The term “radionuclide” or “radioisotope” as used herein includes ions thereof. Thus, for example, the terms lead, Pb, ²¹²Pb or ²⁰³Pb are intended to encompass the ionic form of the radioisotope element.

The terms “chelator” or “chelating agent” refer to polydentate (multiple bonded) ligands capable of forming two or more separate coordinate bonds with (“coordinating”) a central (metal) ion. Specifically, such molecules or molecules sharing one electron pair may also be referred to as “Lewis bases”. The central (metal) ion is usually coordinated by two or more electron pairs to the chelating agent. Usually, the electron pairs of a chelating agent forms coordinate bonds with a single central (metal) ion; however, in certain examples, a chelating agent may form coordinate bonds with more than one metal ion, with a variety of binding modes being possible. The terms “coordinating” and “coordination” refer to an interaction in which one multi-electron pair donor coordinatively bonds (“is coordinated”) to, i.e. shares two or more unshared pairs of electrons with, one central (metal) ion. The chelating agent is preferably chosen based on its ability to coordinate (or bind) the desired central (metal) ion, usually a radionuclide as specified herein. Examples of chelators include DOTA (1,4,7,10-tetraazacyclododecane-1,4,7,10-tetraacetic acid) and DOTAM (1,4,7,10-tetraazacyclododecane-1,4,7,10-tetraacetamide) (also called TCMC), and analogues or derivatives thereof. Such analogues or derivatives include, for example, monoacid forms of TCMC. The term “connector” refers to any chemical moiety that covalently connects a peptide or polypeptide, such as, e.g., an ankyrin repeat protein, such as, e.g., a protein comprising an ankyrin repeat domain with binding specificity for DLL3, with a chelator. Examples of connectors include chemical moieties comprising maleimide or a derivative thereof and chemical moieties comprising phenyl isothiocyanate or a derivative thereof.

Examples of a chelator connected to a connector include p-SCN-Bn-TCMC (2-[4,7,10-tris(2-amino-2-oxoethyl)-6-[(4-isothiocyanatophenyl)methyl]-1,4,7,10-tetrazacyclododec-1-yl]acetamide) (PubChem CID 10076170) and monoacid forms of p-SCN-Bn-TCMC, wherein the chelator TCMC or a monoacid form thereof is connected to a chemical moiety that allows covalent connection to a peptide or polypeptide.

The term “physiological conditions” refers to conditions normally present in a mammalian body. Thus, for example for humans, physiological conditions mean a pH between 7.35 and 7.45, with the average at 7.40, and a temperature between 36.1° C. and 37.2° C., with the average at 37° C.

Embodiments of the present disclosure are further defined in the following Examples. It should be understood that these Examples are given by way of illustration only and that the invention is not restricted to the particular embodiments described in the Examples. From the above discussion and these Examples, one skilled in the art can ascertain the essential characteristics of the present disclosure, and without departing from the spirit and scope thereof, can make various changes and modifications of the embodiments of the disclosure to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims. The disclosure of each reference set forth herein is incorporated herein by reference in its entirety, and for the disclosure referenced herein. This specification refers to a number of amino acid sequences and SEQ ID NOs that are disclosed in the Sequence Table and the associated Sequence Listing, which is herewith incorporated by reference in its entirety.

### EXAMPLES

### Materials

Chemicals were purchased from Sigma-Aldrich (USA). Oligonucleotides were from Microsynth (Switzerland). Unless stated otherwise, DNA polymerases, restriction enzymes and buffers were from New England Biolabs (USA) or Fermentas/Thermo Fisher Scientific (USA). Inducible E. coli expression strains were used for cloning and protein production, e.g. E. coli XL1-blue (Stratagene, USA) or BL21 (Novagen, USA). TEV protease was from Sigma-Aldrich (USA). Double-stranded gene fragments (eBlocks) were obtained from IDT (US). Maleimide DTPA was purchased from Chematech, metal-free PBS was purchased from VWR and Chelex 100 chelating resins were purchased from BioRad.

### Molecular Biology

Unless stated otherwise, methods are performed according to known protocols (see, e.g., Sambrook J., Fritsch E. F. and Maniatis T., Molecular Cloning: A Laboratory Manual, Cold Spring Harbor Laboratory 1989, New York or subsequent editions).

### Designed Ankyrin Repeat Protein Libraries

Methods to generate designed ankyrin repeat protein libraries have been described, e.g. in U.S. Pat. No. 7,417,130; Binz et al. 2003, loc. cit.; Binz et al. 2004, loc. cit. By such methods designed ankyrin repeat protein libraries having randomized ankyrin repeat modules and/or randomized capping modules can be constructed. For example, such libraries could accordingly be assembled based on a fixed N-terminal capping module or a randomized N-terminal capping module, one or more randomized repeat modules, and a fixed C-terminal capping module or a randomized C-terminal capping module (see, e.g., the N-terminal capping modules and C-terminal capping modules provided in WO2021116462 and WO2021116469). Preferably, such libraries are assembled to not have any of the amino acids C, G, M, N (in front of a G residue) and P at randomized positions of repeat or capping modules.

Furthermore, such randomized modules in such libraries may comprise additional polypeptide loop insertions with randomized amino acid positions. Examples of such polypeptide loop insertions are complement determining region (CDR) loop libraries of antibodies or de novo generated peptide libraries. For example, such a loop insertion could be designed using the structure of the N-terminal ankyrin repeat domain of human ribonuclease L (Tanaka, N., Nakanishi, M, Kusakabe, Y, Goto, Y., Kitade, Y, Nakamura, K. T., EMBO J. 23(30), 3929-3938, 2004) as guidance. In analogy to this ankyrin repeat domain where ten amino acids are inserted in the beta-turn present close to the border of two ankyrin repeats, ankyrin repeat protein libraries may contain randomized loops (with fixed and randomized positions) of variable length (e.g. 1 to 20 amino acids) inserted in one or more beta-turns of an ankyrin repeat domain.

The design of such an ankyrin repeat protein library may be guided by known structures of an ankyrin repeat domain interacting with a target. Examples of such structures, identified by their Protein Data Bank (PDB) unique accession or identification codes (PDB-IDs), are 1WDY, 3V31, 3V30, 3V2X, 3V20, 3UXG, 3TWQ-3TWX, 1N11, 1S70 and 2ZGD.

Examples of designed ankyrin repeat protein libraries, such as N2C and N3C designed ankyrin repeat protein libraries, have been described (U.S. Pat. No. 7,417,130; Binz et al. 2003, loc. cit.; Binz et al. 2004, loc. cit.). The digit in N2C and N3C describes the number of randomized repeat modules present between the N-terminal and C-terminal capping modules.

### Example 1: Ankyrin Repeat Proteins with Binding Specificity for DLL3 and their Kinetic Binding Parameters and Binding Affinities

Ankyrin Repeat Domains with Binding Specificity for DLL3 and Generation of Half-Life Extended DLL3-Specific Binding Proteins

Ankyrin repeat domains with binding specificity for DLL3 have been described in U.S. 63/627,705 and U.S. 63/550,951. Examples of such DLL3-specific ankyrin repeat domains include SEQ ID NOs: 1 to 4. Ankyrin repeat domains with binding specificity for human serum albumin (HSA) have been described previously, e.g. in WO 2020/245171. Examples of such serum albumin-specific ankyrin repeat domains include SEQ ID NOs: 5 to 7. Linking a HSA-specific ankyrin repeat domain to an ankyrin repeat protein without binding specificity for HSA may extend the serum half-life of such ankyrin repeat protein. Applicant investigated the effects of linking a HSA-specific ankyrin repeat domain to an ankyrin repeat protein with binding specificity for DLL3 on the biological properties of ankyrin repeat proteins with binding specificity for DLL3.

In order to generate half-life extended DLL3-specific ankyrin repeat proteins, nucleic acid sequences encoding the DLL3-specific ankyrin repeat domains of SEQ ID NOs: 1 to 4 were first subcloned C-terminally of an expression cassette consisting of a His-tag and a TEV cleavage site (SEQ ID NO: 20), a serum albumin-specific ankyrin repeat domain (SEQ ID NO: 7) and a peptide linker (SEQ ID NO: 8). Examples of resulting constructs comprising two ankyrin repeat domains (2D), one with binding specificity for DLL3 and one with binding specificity for serum albumin, include SEQ ID NOs: 11 to 14. The resulting constructs comprising two ankyrin repeat domains were expressed in E. coli and purified using the N-terminal His-tag according to standard protocols.

### Kinetic Binding Parameters and Binding Affinities of Recombinant Ankyrin Repeat Proteins

Kinetic binding parameters and binding affinities of two domain (2D) ankyrin repeat proteins comprising an ankyrin repeat domain with binding specificity for DLL3 and an ankyrin repeat domain with binding specificity for serum albumin were determined by surface plasmon resonance (SPR) multi-trace analysis.

In brief, a SPR assay was used to determine the binding kinetics to the target human DLL3. The data were generated using BioRad ProteOn instrument in SPR running buffer (PBS, pH 7.4 containing 0.005% Tween 20®). A SAHC200M chip (Xantec) was conditioned according to the manufacturer's protocol. The chip was coated with biotinylated target (bio-DLL3) to reach a signal intensity of 1780 RU. All analytes (3-fold dilution ranging from 300-33.3 nM) were injected in succession for 240 s (100 μl/min) and dissociation was recorded for 10800 s (100 μl/min). Each injection was followed by a regeneration step with 16 mM H₃PO₄ for 18 s. The data was double referenced (control spot and buffer injection) and fitted to a 1:1 Langmuir model. The bio-DLL3 target used was as described in U.S. 63/550,951, i.e. the extracellular domain (ECD) of human DLL3 protein (Q9NYJ7, Gene ID: 10683, residues 27 to 466; purchased from Evitria) linked via an Avi-tag (for site-directed biotinylation), followed by a 3C protease recognition site, to a Fc knob into hole (hDLL3 ECD-Avi-Fcₖᵢₕ). The obtained SPR traces were used to determine the ankyrin repeat protein-DLL3 interaction parameters, summarized in Table 1.

Similarly, a SPR assay was used to determine the binding kinetics to human or mouse serum albumin. The data were generated using a Bruker Sierra SPR-32-pro instrument with SPR running buffer (PBS, pH 7.4 containing 0.005% Tween 20®). A HC200M chip (Xantec) was conditioned according to the manufacturer's protocol. The chip was coated with purified human serum albumin (HSA, CSL) or mouse serum albumin (MSA, Sigma-Aldrich) in NaOAc pH5.0 to reach a signal intensity of ˜900 (HSA) or ˜1500 (MSA) RU. All analytes (3-fold dilution ranging from 1000-12.35 nM) were injected in succession for 240 s (25 μl/min) and dissociation was recorded for 600 s (25 μl/min). The data was double referenced (control spot and buffer injection) and fitted to a 1:1 Langmuir model. The obtained SPR traces were used to determine the ankyrin repeat protein-serum albumin interaction parameters, summarized in Table 2.

Results are shown for binding to recombinant human DLL3 in Table 1 and for binding to human or mouse serum albumin in Table 2.

TABLE 1

| DARPin | kₒₙ [1/Ms] | k_{off} [1/s] | K_{D} [M] |
|---|---|---|---|
| MAM279 | 2.33E+04 | 1.44E−05 | 6.16E−10 |
| MAM283 | 1.88E+04 | 1.79E−05 | 9.53E−10 |
| MAM160 | 1.01E+04 | 2.01E−05 | 1.99E−09 |
| MAM282 | 1.25E+04 | 3.79E−05 | 3.03E−09 |

TABLE 2

| | HSA | | | MSA | | |
|---|---|---|---|---|---|---|
| DARPin | Kₒₙ [1/Ms] | K_{off} [1/s] | K_{D} [M] | Kₒₙ [1/Ms] | K_{off} [1/s] | K_{D} [M] |
| MAM279 | 1.23E+05 | 9.23E−03 | 7.50E−08 | 9.124E+004 | 3.35E−02 | 3.67E−07 |
| MAM283 | 8.95E+04 | 9.24E−03 | 1.03E−07 | 7.468E+004 | 3.78E−02 | 5.07E−07 |
| MAM160 | 1.10E+05 | 1.05E−02 | 9.59E−08 | 8.043E+004 | 2.71E−02 | 3.36E−07 |
| MAM282 | 1.20E+05 | 8.89E−03 | 7.43E−08 | 1.059E+005 | 3.54E−02 | 3.35E−07 |

The SPR experiments confirmed that the selected ankyrin repeat proteins bind to human DLL3 as well as to human or mouse serum albumin, with the kinetic properties and affinities shown in Tables 1 and 2.

### Example 2: Binding of Ankyrin Repeat Proteins to Cells Expressing DLL3 on their Surface

Cell binding titration assays were performed for selected His-tagged single domain (1 D) and two domain (2D) designed ankyrin repeat proteins to determine binding to human DLL3 expressed on HEK293T-huDLL3 cells in absence and presence of human serum albumin. Controls included Rova antibody as a positive control and a non-binding DARPin comprising SEQ ID NO: 21 as a negative control. HEK293T-hDLL3 cells are HEK293T cells that were stably transfected to express human DLL3 on their surface.

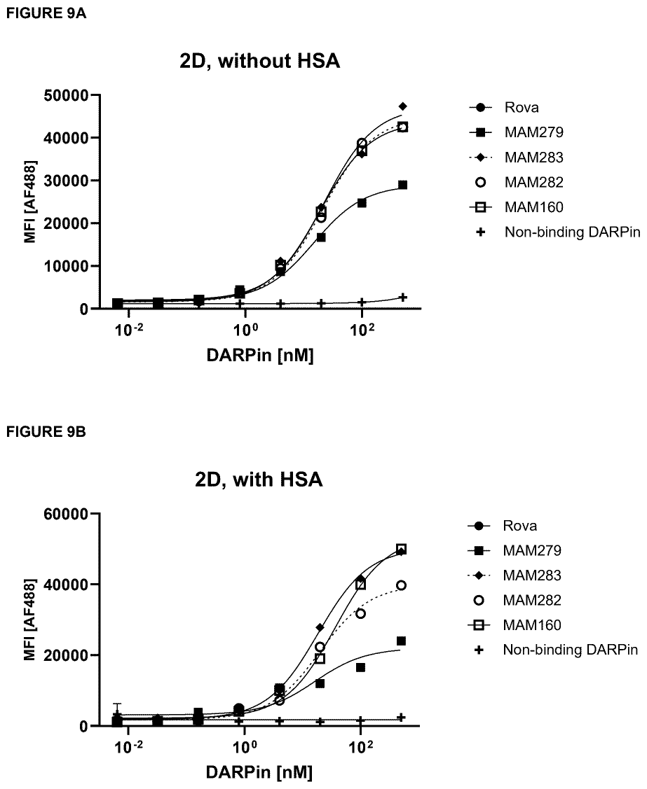

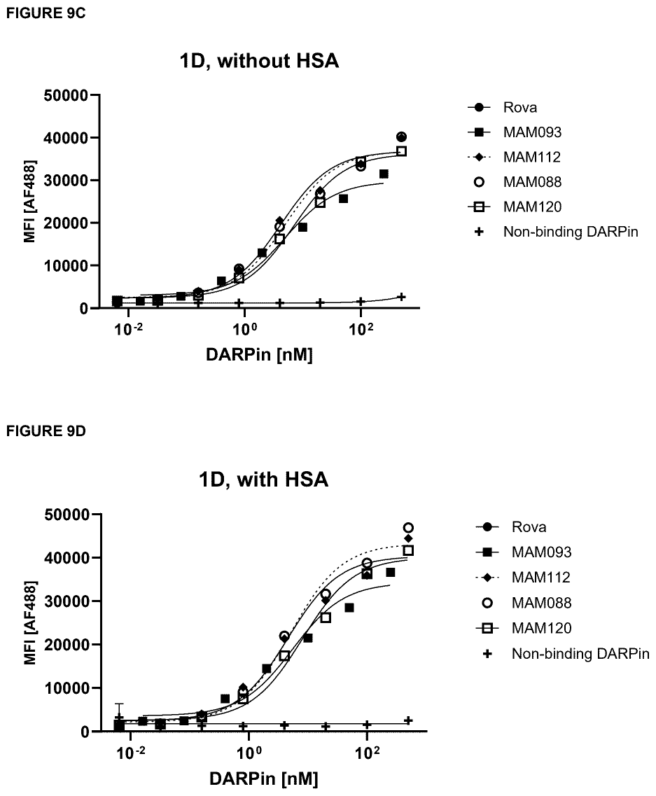

In brief, 1×10⁵ cells/well were resuspended in 100 μl DARPin dilution (500 nM, 1:5 serial diluted) and incubated for 1 h at 4° C. Cells were washed twice with PBS and resuspended in 100 μl anti-DARPin antibody (rabbit anti-DARPin 1.4.8) and LIVE/DEAD fixable cell stain (1:1000, Thermo Fisher #L34957). This step was performed in absence and presence of 10 pM Human Serum Albumin (CSL Behring #150570). After incubation and washing, cells were incubated with detection antibody for 30 minutes (Goat anti-Human IgG (H+L), AF647, Thermo Fisher #21445). Cells were fixed (Paraformaldehyde Solution, LucernaChem) and acquired at Attune N×T (Thermo Life Technologies). Raw fcs files were exported and analyzed using FlowJo software. MFI values of live AF647-positive cells were exported from FlowJo and plotted using GraphPad Prism software. None of the tested ankyrin repeat proteins show binding to the HEK293T wildtype cells. Results are shown in FIGS. 9A to 9D and summarized in Table 3.

TABLE 3

| | Cell binding | Cell binding |
|---|---|---|
| DARPin | EC50; w/o HSA | EC50; with HSA |
| (or control) | [nM] | [nM] |
| “Rova” control (MPEXT268) | 0.1 | 0.3 |
| MAM279 | 14.9 | 16.3 |
| MAM283 | 22.1 | 18.4 |
| MAM160 | 18.9 | 36.6 |
| MAM282 | 21.2 | 19.6 |
| MAM093 | 4.9 | 5.5 |
| MAM112 | 4.2 | 4.6 |
| MAM120 | 7 | 8 |
| MAM088 | 5 | 5.1 |
| Non-binding DARPin control | n.a. | n.a. |

The cell binding titration assays confirmed that the selected ankyrin repeat proteins bind to human DLL3 expressed on cells, both in absence and presence of human serum albumin.

Furthermore, cell binding titration assays were also performed with selected two domain (2D) designed ankyrin repeat proteins to determine binding to human cells expressing DLL3 endogenously, in absence and presence of human serum albumin. For this purpose, NCI-H82 cells were used, a human lung carcinoma cell line which endogenously expresses DLL3 on the surface. The designed ankyrin repeat proteins had a tag (SEQ ID NO: 20) at their N-terminal end for ease of purification. A non-binding DARPin comprising SEQ ID NO: 22 was used as a negative control.

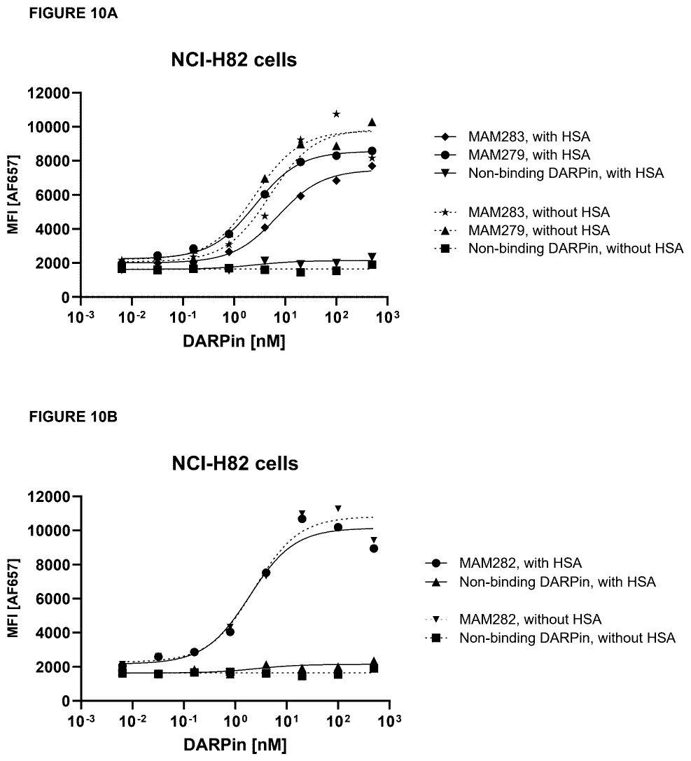

In brief, 1×10⁵ cells/well were resuspended in 100 μl DARPin dilution (500 nM, 1:5 serial diluted in PBS, 2% FBS) and incubated for 1 h at 4° C. This step was performed in absence and presence of 10 μM Human Serum Albumine (CSL Behring #150570). Cells were washed twice with PBS, 2% FBS and resuspended in 100 μl anti-DARPin antibody (rabbit anti-DARPin 1.4.8) and LIVE/DEAD cell stain (1:1000, Biolegend #423106). After incubation and washing, cells were incubated with detection antibody for 30 minutes (Goat anti-Human IgG (H+L), AF647, Thermo Fisher #21445). Cells were fixed (Paraformaldehyde Solution, TCL119, LucernaChem) and acquired at Attune N×T (Thermo Life Technologies). Raw fcs files were exported and analyzed using FlowJo software. MFI values of live AF647-positive cells were exported from FlowJo and plotted using GraphPad Prism software. Results are shown in FIGS. 10A to 10B and summarized in Table 11.

TABLE 11
Binding of selected DARPins to NCI-H82 cells,
in absence and presence of human serum albumin

| | Cell binding | Cell binding |
|---|---|---|
| DARPin | EC50; w/o HSA | EC50; with HSA |
| (or control) | [nM] | [nM] |
| MAM279 | 2.5 | 2.1 |
| MAM283 | 5.4 | 8.1 |
| MAM282 | 2.6 | 2.6 |
| Non-binding DARPin control | n.a. | n.a. |

The cell binding titration assays demonstrated that the selected ankyrin repeat proteins bind to DLL3 endogenously expressed on human cells, such as lung carcinoma cells, both in absence and presence of human serum albumin.

### Example 3: Biodistribution of Radio-Labelled DARPin Conjugates in NCI-H82 Tumor Model

Generation of DARPins with a C-Terminal Cysteine for Site-Specific Conjugation and Radio-Labeling

Proteins were produced at high quality for biodistribution (BioD) in vivo experiments. Various designed ankyrin repeat proteins (such as, e.g., MAM120 and MAM160) were subcloned into a derivative of the pQE30 (Qiagen) expression vector (pMPDV025), generating constructs containing a His-TEV tag (SEQ ID NO: 20) fused to the N-terminus of the subcloned ankyrin repeat protein (having a N-terminal GS), and a C-terminal cysteine-containing tag (SEQ ID NO: 10) fused to the C-terminus of the subcloned ankyrin repeat protein. These ankyrin repeat protein constructs were expressed in E. coli and purified over a Ni-NTA column before desalting over a HiLoad 26/600 Superdex 200 column. Main fractions were pooled, digested by TEV protease (Sigma Aldrich) on a roller shaker at room temperature (RT) overnight (˜18-20h). Samples were taken after overnight digestion and analyzed by SDS-PAGE to assess the degree of cleavage. Non-cleaved DARPins still containing the His-tag as well as the His-tagged TEV protease were removed by incubating for 30 min with Ni-NTA resin on a roller shaker at RT, before centrifugation and removal of the Ni-NTA resins by decanting and filtration on empty columns. Supernatant/flow-through was incubated with 5 mM TCEP for 20 min and purified by size-exclusion chromatography, before up-concentration. Final purified samples were stored in 50 mM NaPO4, 150 mM NaCl, pH 6.5. Detailed methods for the production and purification of proteins are well known to the practitioner in the art. Examples of resulting DARPins with a C-terminal Cysteine include SEQ ID NOs: 15 to 18.

### Site-Specific Conjugation and Radio-Labeling of DARPins Having a C-Terminal Cysteine

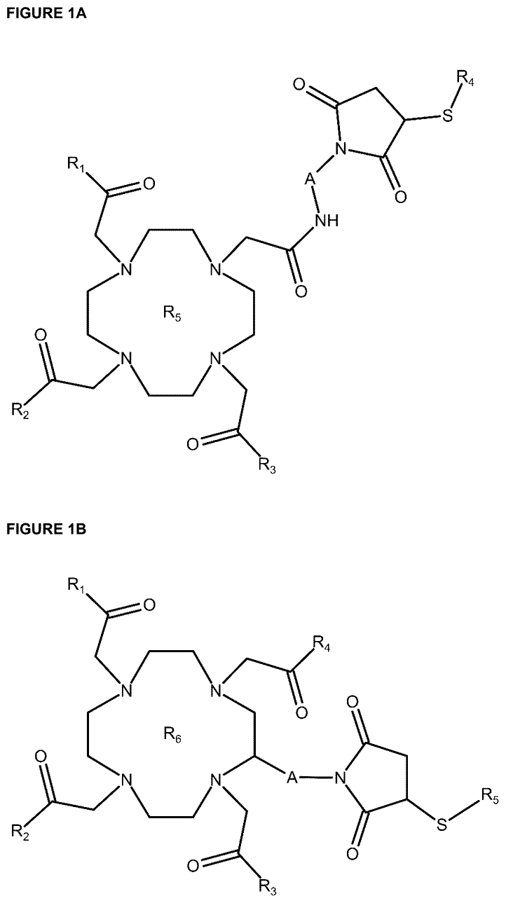

DARPins having a C-terminal Cysteine were conjugated to a chelator (DOTA or DOTAM, or an analogue or derivative thereof) using a connector comprising maleimide. Resulting DARPin-chelator conjugates were then labeled with lead, such as Pb-212. Methods for the generation and purification of Pb-203 or Pb-212, for the conjugation of chelators, such as DOTA, DOTAM, or analogues or derivatives thereof, to (poly)peptides, and for the subsequent radio-labeling of chelator-(poly)peptide conjugates with lead, such as Pb-203 or Pb-212, have been described (see, e.g., US 2022/0037046; US 2023/0372552; U.S. Pat. No. 11,541,133; Baidoo et al., Nucl Med Biol. 2013 July, 40(5): 592-599; McNeil et al., Nature Scientific Reports (2023) 13:10623; WO 2017/220767). The synthesis of chelators, such as DOTAM, has also been described (see, e.g., Maumela et al., J. Am. Chem. Soc. 1995, 117, 6698-6707). Furthermore, methods for using a maleimide-comprising connector to connect a cysteine-comprising polypeptide (e.g. an ankyrin repeat protein) to a chemical moiety have been described (see, e.g., WO 2011/135067). The structures of resulting radio-labeled DARPin conjugates are illustrated by the examples shown in FIGS. 1A and 1B (whereby the protein moieties R4 in FIG. 1A and R5 in FIG. 1B vary depending on the DARPin used for the conjugation). Control protein conjugates (e.g. of Rova antibody) used in some experiments were generated similarly as the radio-labeled DARPin conjugates.

In an exemplary method used herein, DARPin protein (after purification by chromatography steps; concentration: 3-5 g/L UV₂₈₀) was reduced with Tris-(2-carboxyethyl)-phosphine (TCEP) (5 mM final TCEP concentration in the protein solution) for one hour at RT (20-25° C.) while circulating in a tangential flow filtration (TFF)-system. The reduced protein solution was concentrated to 5 g/L (UV₂₈₀) by ultrafiltration deploying a TFF-membrane with a molecular weight cut-off of 10 kDa. The TCEP was removed by diafiltration against 6 turnover volumes of conjugation buffer (50 mM NaPO4, 150 mM NaCl, pH 6.5) prior to chelator (e.g. DOTAM) conjugation. DOTAM-Maleimide (Macrocyclics, Product B-382; Chemical name: 1,4,7,10-Tetraazacyclododecane-1,4,7-tris(carbamoylmethyl)-10-maleimidoethylacetamide) was dissolved in conjugation buffer and added to the TCEP-free retentate in the TFF-system to reach a chelator excess factor of 1.5 over DARPin protein. The conjugation reaction solution was circulated for one hour at RT in the TFF-system. Unconjugated chelator was removed by diafiltration against 7 turnover volumes of conjugation buffer. The DARPin-chelator solution was recovered from the TFF-system and diluted with conjugation buffer to 2 g/L total protein (UV₂₈₀), filtered through a 0.22-micron filter and stored at ≤−60° C. until loading with a radioactive isotope. For loading of the conjugate with a radionuclide, for example Pb-212 and the DARPin-chelator conjugate were mixed in buffer (pH 5.0) at a ratio of 10 μCi of Pb-212 per 14.7 pmol of DARPin-chelator conjugate and incubated at RT for at least 10 minutes. The method resulted in a conjugate having a structure of Formula (VI), wherein all three of R1, R2 and R3 in Formula (VI) are NH₂ and the A in Formula (VI) is —CH₂—CH₂—.

### Biodistribution of Radio-Labeled DARPin Conjugates Comprising MAM120 or MAM160

Biodistribution of radio-labeled DARPin conjugates was investigated in a NCI-H82 mouse tumor model.

NCI-H82 tumor model. R2G2 mice were purchased from Envigo. All studies were conducted under the approval of the institutional IACUC committee. Animals were maintained under specific-pathogen-free (SPF) conditions with daily cycles of light and darkness (12 h/12 h), in line with ethical guidelines. No manipulations were performed during the first 5 days after arrival, to allow the animals to acclimatize to the new environment. All mice were monitored daily for assessment of physical condition and general well-being. NCI-H82 cells were purchased from ATCC (Catalog No. HTB-175). Solid xenografts were established by subcutaneous (SQ) injection of NCI-H82 cells in RPMI media mixed 1:1 with Corning® Matrigel® basement membrane matrix (GFR; Corning, Cat No 354230.). Cells were quantified using Countess cell counter. Each mouse was injected SQ with 5×10⁶ NCI-H82 cells in 100 μL RPMI/GFR-Matrigel into the right flank of each mouse. Tumor volumes were estimated three times per week through calipering, according to the formula: volume=0.5×length×width² and grown until they reached 200-300 mm³. Tumor-associated antigen level expressed in tumors, tumor vascularization profile and DLL3 soluble form in mouse serum was tested.

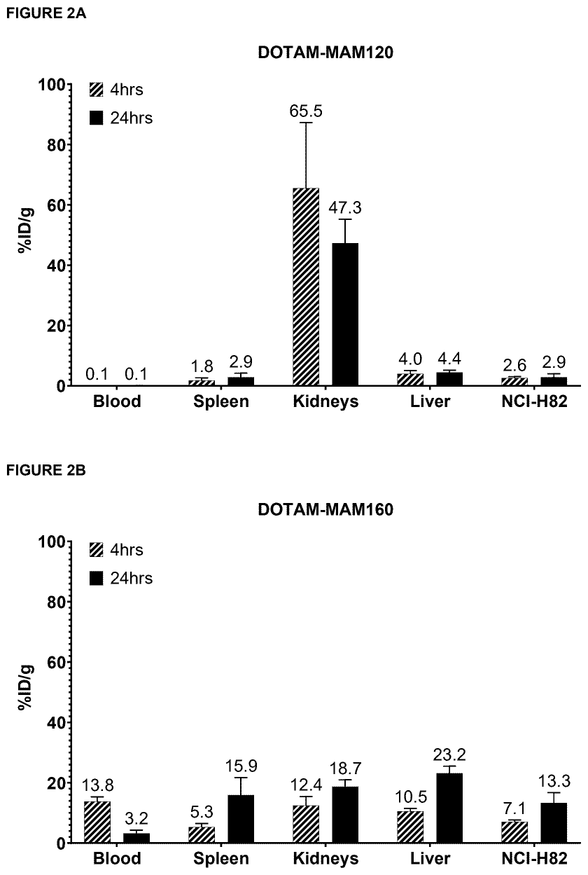

Biodistribution study. DARPins were conjugated with chelator (DOTAM) and labeled with Pb-212 as described above. Radio-labeled (Pb-212) DARPin conjugates were then injected at 0.01 mg/kg (10 μCi DARPin conjugates) into the tail vein of R2G2 mice (females) xenografted subcutaneously (at age of about 7 weeks) with NCI-H82 cells (5 million cells, growth factor reduced Matrigel) and bearing tumors (5-10 mm in diameter). Based on immunohistochemistry analyses, the NCI-H82 tumors are considered representative of human DLL3 expression found in patients with small cell lung cancer (data not shown). Biodistribution was monitored at 4 h and 24h post-injection. Mice were euthanized by CO2 inhalation and cervical dislocation. Blood, spleen, kidneys, liver and tumor were extracted, weighed and the radioactivity was determined with a γ-counter counter (such as Wizard²2470, Perkin Elmer). The data were expressed as the % injected dose/gram (% ID/g). The results are shown in FIGS. 2A and 2B and Table 4.

TABLE 4

| | |
|---|---|
| MAM120 | MAM160 |

| % ID/g | 4 h | 24 h | 4 h | 24 h |
|---|---|---|---|---|
| Blood | 0.1 | 0.1 | 13.8 | 3.2 |
| Kidneys | 65.5 | 47.3 | 12.4 | 18.7 |
| Tumors | 2.6 | 2.9 | 7.1 | 13.3 |
| T:K ratio | 0.04 | 0.06 | 0.58 | 0.71 |

MAM120 conjugate showed a low tumor to kidney (T:K) ratio at 4 and 24h in NCI-H82 tumors. With the addition of an HSA-binding DARPin with a relatively high binding affinity, the T:K increased for both timepoints. MAM160 conjugate showed a higher exposure in blood, a lower uptake in the kidneys, and a better tumor penetration and accumulation (see Table 4).

### Investigation of the Effect of Different Binding Affinities to Serum Albumin

Three different HSA-specific ankyrin repeat domains were tested in combination with MAM120 in two domain DARPin constructs. These three HSA-specific ankyrin repeat domains differed in their binding affinity for HSA, and were termed Low affinity HSA binding DARPin, Intermediate affinity HSA binding DARPin, and High affinity HSA binding DARPin (SEQ ID NO: 7). Two domain (2D) DARPin constructs without a His-tag and with a C-terminal Cysteine were generated as described above. These 2D DARPin constructs were identical to each other, except that they differed in their HSA-specific ankyrin repeat domain. The 2D DARPin construct comprising the High affinity HSA binding DARPin corresponded to SEQ ID NO: 17, which is MAM160 with a GS at the N-terminus and a Cysteine-containing tag at the C-terminus. The three 2D DARPin constructs were termed Low HSA affinity 2D DARPin, Intermediate HSA affinity 2D DARPin, and High HSA affinity 2D DARPin (SEQ ID NO: 17). They were investigated for binding properties and biodistribution.

SPR Multi-Trace Analysis of the Interaction with Human or Mouse Serum Albumin

For SPR analysis, the free Cysteine of the 2D DARPins was capped with Iodoacetamide (IAM). A surface plasmon resonance (SPR) assay was then used to determine the binding kinetics of the three 2D DARPins to human or mouse serum albumin. Various concentrations of 2D DARPins starting from 2000 nM were applied to immobilized human serum albumin (HSA) or mouse serum albumin (MSA) for on-rate and off-rate measurements.

The data were generated using a BioRad ProteOn instrument with SPR running buffer (PBS, pH 7.4 containing 0.05% Tween 20®). A HC200M chip (Xantec) was conditioned according to the manufacturer's protocol. The chip was coated with purified human serum albumin (HSA, CSL) or mouse serum albumin (MSA, Sigma-Aldrich) in NaOAc pH5.0 to reach a signal intensity of ˜380 (HSA) or ˜630 (MSA) RU for the analysis of Intermediate HSA affinity 2D DARPin and High HSA affinity 2D DARPin; ˜1125 (HSA) or ˜1965 (MSA) for the analysis of Low HSA affinity 2D DARPin. All analytes (3-fold dilution ranging from 2000-24.69 nM) were injected in succession for 240 s (50 μl/min) and dissociation was recorded for 600 s (50 μl/min). The data was double referenced (control spot and buffer injection) and fitted to a 1:1 Langmuir model. The obtained SPR traces were used to determine binding parameters. The binding parameters of the three 2D DARPins to human and mouse serum albumin are summarized in Table 12.

TABLE 12

| | HSA | | | MSA | | |
|---|---|---|---|---|---|---|
| DARPin | Kₒₙ [1/Ms] | K_{off} [1/s] | K_{D} [M] | Kₒₙ [1/Ms] | K_{off} [1/s] | K_{D} [M] |
| High HSA affinity 2D | 1.05E+05 | 3.11E−02 | 2.95E−07 | 1.33E+05 | 3.45E−02 | 2.59E−07 |
| DARPin | | | | | | |
| Intermediate HSA affinity | 7.63E+04 | 3.94E−02 | 5.17E−07 | 6.57E+04 | 4.08E−02 | 6.21E−07 |
| 2D DARPin | | | | | | |
| Low HSA affinity 2D | 6.20E+04 | 1.28E−01 | 2.07E−06 | No | No | No |
| DARPin | | | | binding | binding | binding |

### Biodistribution (Tumor to Kidney Ratio) of Radio-Labeled DARPin Conjugates Comprising MAM120 or One of the Three 2D DARPins

DARPins were conjugated with chelator (DOTAM) and labeled with Pb-212 as described above. Radio-labeled (Pb-212) DARPin conjugates were then injected at 0.01 mg/kg (10 μCi DARPin conjugates) into the tail vein of R2G2 mice (females) xenografted subcutaneously (at age of about 7 weeks) with NCI-H82 cells (5 million cells, growth factor reduced Matrigel) and bearing tumors (5-10 mm in diameter). Biodistribution was monitored at 4 h and 24h post-injection. Mice were euthanized by CO2 inhalation and cervical dislocation. Kidneys and tumor were extracted, weighed and the radioactivity was determined with a γ-counter counter (such as Wizard²2470, Perkin Elmer). The data were expressed as the % injected dose/gram (% ID/g) and the tumor to kidney (T:K) ratio was determined. The results are summarized in Table 5.

TABLE 5

| | HSA | |
|---|---|---|
| DARPin and timepoint | affinity | T:K |
| MAM120_4 hrs | n.a. | 0.04 |
| MAM120_24 hrs | | 0.06 |
| Low affinity HSA binding | Low | 0.01 |
| DARPin - MAM120_4 hrs | | |
| Low affinity HSA binding | | 0.01 |
| DARPin - MAM120_24 hrs | | |
| Intermediate affinity HSA | Mid | 0.31 |
| binding DARPin - MAM120_4 hrs | | |
| Intermediate affinity HSA | | 0.29 |
| binding DARPin - MAM120_24 hrs | | |
| High affinity HSA binding DARPin | High | 0.58 |
| (SEQ ID NO: 7) - MAM120_4 hrs (MAM160) | | |
| High affinity HSA binding DARPin | | 0.71 |
| (SEQ ID NO: 7) - MAM120_24 hrs (MAM160) | | |

Taken together, the tested 2D DARPins displayed different binding affinities to human or mouse serum albumin. The binding affinities to HSA were relatively low (Ko of 2.07×10⁻⁶ M), intermediate (Ko of 5.17×10⁻⁷ M), and relatively high (Ko of 2.95×10⁻⁷ M). Thus, the High HSA affinity 2D DARPin had an about 10-fold higher binding affinity to HSA than the Low HSA affinity 2D DARPin. The T:K ratio of the radio-labelled conjugates increased with increasing affinity for HSA (see Table 5). The addition of the low affinity HSA-binding ankyrin repeat domain did not result in any increase of the T:K ratio as compared to the MAM120 conjugate. In contrast, the addition of the intermediate affinity HSA-binding ankyrin repeat domain resulted in a significantly increased T:K ratio. The highest T:K ratio was achieved for the MAM160 conjugate, which had the highest affinity for HSA among the tested conjugates.

### Example 4: Biodistribution of Radio-Labelled MAM160 Conjugate in hDLL3-MC38 Tumor Model

Biodistribution of radio-labeled DARPin conjugates was investigated in a hDLL3-MC38 mouse tumor model.

hDLL3-MC38 tumor model. ATH mice were purchased from Envigo. General handling and maintenance of mice was done as described above for the NCI-H82 tumor model. hDLL3-MC38 cells were purchased from Biocytogen (Catalog No. 311448). Solid xenografts were established by subcutaneous (SQ) injection of hDLL3-MC38 cells in RPMI media mixed 1:1 with Corning® Matrigel® basement membrane matrix (GFR; Corning, Cat No 354230.). Cells were quantified using Countess cell counter. Each mouse was injected SQ with 5×10⁶ hDLL3-MC38 cells in 100 μL RPMI/GFR-Matrigel into the right flank of each mouse. Tumor volumes were estimated three times per week through calipering, according to the formula: volume=0.5×length×width² and grown until they reached 200-300 mm³. Tumor-associated antigen level expressed in tumors, tumor vascularization profile and DLL3 soluble form in mouse serum was tested.

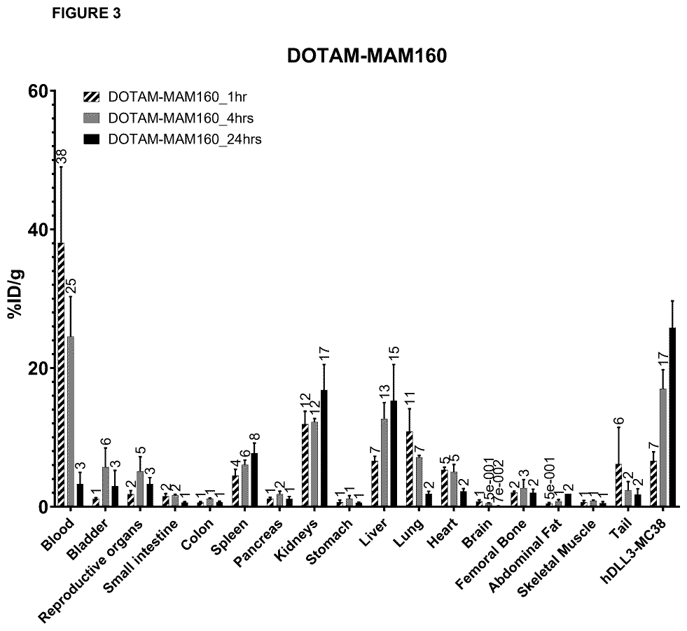

Biodistribution study. DARPins were conjugated with chelator (DOTAM) and labeled with Pb-212 as described in Example 3. Radio-labelled (Pb-212) DARPin conjugates were then injected at 0.01 mg/kg (10 μCi DARPin conjugates) into the tail vein of athymic mice (females) xenografted subcutaneously (at age of about 7 weeks) with hDLL3-MC38 cells (5 million cells, growth factor reduced Matrigel) and bearing tumors (5-10 mm in diameter). hDLL3-MC38 cells are MC38 cells which have been stably transfected to express human DLL3. Based on immunohistochemistry analyses, the hDLL3-MC38 tumors have a higher level of human DLL3 expression than the NCI-H82 tumors described in Example 3 (data not shown). Biodistribution was monitored at 1 h, 4 h and 24h post-injection. Mice were euthanized by CO2 inhalation and cervical dislocation. Blood, bladder, reproductive organs, small intestine, colon, spleen, pancreas, kidneys, stomach, liver, lung, heart, brain, femoral bone, abdominal fat, skeletal muscle, tail and tumor were extracted, weighed and the radioactivity was determined with a γ-counter (such as Wizard²2470, Perkin Elmer). The data were expressed as the % injected dose/gram (% ID/g). The results are shown in FIG. 3 and Table 6.

TABLE 6

| | MAM160 | | |
|---|---|---|---|
| % ID/g | 1 h | 4 h | 24 h |
| Blood | 38 | 25 | 3 |
| Kidneys | 12 | 12 | 17 |
| Tumors | 7 | 17 | 26 |
| T:K ratio | 0.58 | 1.42 | 1.53 |

Half Life Extension (HLE) by SEQ ID NO: 7 allowed T:K >1 at 4 h and 24h for MAM120 in MC38-hDLL3 tumors. No unexpected uptake in other organs was observed (e.g. no major uptake observed in femurs, a potential site for bone marrow toxicity) (see Table 6 and FIG. 3).

### Example 5: Efficacy of Radio-Labelled MAM160 Conjugates in hDLL3-MC38 Tumor Model

To determine the efficacy of radio-labelled (Pb-212) DARPin (MAM160) conjugate in inhibiting the growth of tumors that express DLL3, different dose regimens (single or repeated dose) of the conjugate were tested in a mouse tumor model (hDLL3-MC38).

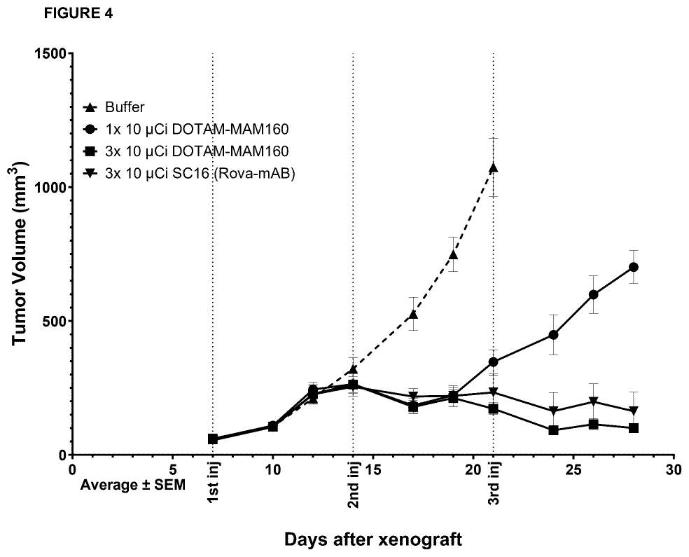

DARPins (or Rova antibody) were conjugated with chelator (DOTAM) and labeled with Pb-212 as described in Example 3. Radio-labelled (Pb-212) DARPin conjugates and Rova conjugate (used as control molecule) were then injected at 1×10 μCi or 3×10 μCi (1 week apart) into the tail vein of athymic mice (females) xenografted subcutaneously (at age of about 7 weeks) with hDLL3-MC38 cells. The first injections were done 7 days after the xenografts. Animals were under observation daily and 3× per week tumors were measured by caliper. Animals were sacrificed when tumors reached 1000 mm³ or if termination criteria were met. The data were expressed as average+/−SEM of tumor volume in mm³. Results are shown in FIG. 4 and Table 7.

TABLE 7

| | Mean | Below | | Adjusted |
|---|---|---|---|---|
| | Diff. | threshold? | Summary | P Value |
| Dunnett's multiple | | | | |
| comparisons test | | | | |
| Buffer vs. 1 × 10μCi | 117.3 | No | ns | 0.472 |
| DOTAM-MAM160 | | | | |
| Buffer vs. 3 × 10μCi | 282.6 | Yes | * | 0.017 |
| DOTAM-MAM160 | | | | |
| Buffer vs. 3 × 10μCi Rova | 251.6 | Yes | * | 0.037 |
| Tukey's multiple | | | | |
| comparisons test | | | | |
| 1 × 10μCi DOTAM- | −117.3 | No | ns | 0.630 |
| MAM160 vs. Buffer | | | | |
| 3 × 10μCi DOTAM- | −282.6 | Yes | * | 0.032 |
| MAM160 vs. Buffer | | | | |
| Buffer vs. 3 × 10μCi Rova | 251.6 | No | ns | 0.066 |
| 1 × 10μCi | 165.4 | No | ns | 0.262 |
| DOTAM-MAM160 vs. 3 × | | | | |
| 10 μCi DOTAM-MAM160 | | | | |
| 1 × 10μCi | 134.4 | No | ns | 0.439 |
| DOTAM-MAM160 vs. | | | | |
| 3 × 10μCi Rova | | | | |
| 3 × 10μCi | −30.98 | No | ns | 0.985 |
| DOTAM-MAM160 vs. | | | | |
| 3 × 10μCi Rova | | | | |

The radio-labelled MAM160 conjugate showed efficacy in hDLL3-MC38 tumors equivalent to antibody benchmark (Rova). Statistical analysis done by Dunnett's and Turkeys comparison tests showed that MAM160 conjugate at 3×10 μCi had a significant effect in hDLL3-MC38 tumors (see Table 7).

### Example 6: Dose Response Finding Profile of Radio-Labelled MAM160 Conjugates in CD1 Mice

To determine the dose range of radio-labelled (Pb-212) DARPin (MAM160) conjugates that can be tolerated by mice, different doses of the conjugates were evaluated for potential toxicity in mice.

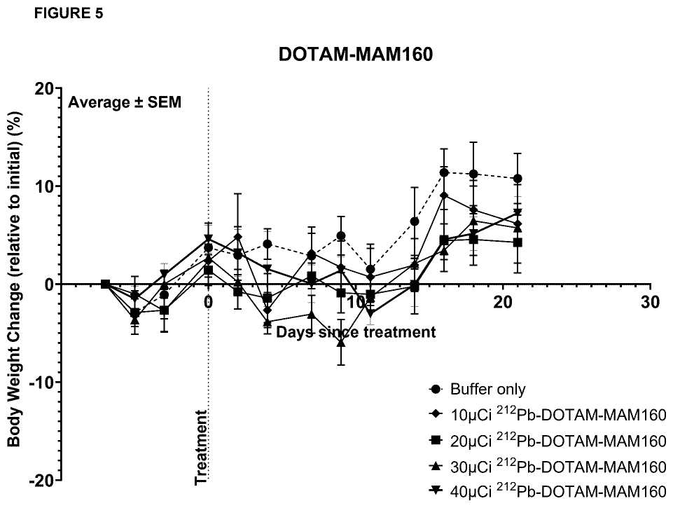

In brief, DARPins were conjugated with chelator (DOTAM) and labeled with Pb-212 as described in Example 3. Radio-labelled (Pb-212) DARPin conjugates (and buffer only as control) were then injected once at 10 μCi, 20 μCi, 30 μCi and 40 μCi into the tail vein of WT CD1 mice (about 7 weeks old females). Animals were under observation daily and 3× per week animals were weighed. Animals were sacrificed 3 weeks after the first injection or if termination criteria were met (15% weight loss over 2 days or 20% from initial weight, lack of grooming over 5 days, lethargy/weakness over 3 days, reduced motility, hunched back, diarrhea, hypothermia, or the combination of multiple criteria). The data were expressed as % of body weight (BW) change (relative to the initial BW −7 day before the treatment). Results are shown in FIG. 5.

All animals appeared healthy and active. Minimal weight loss occurred after injection which was rapidly recovered. All animals gained weight after Day 10 even at doses up to 40 μCi.

### Example 7: Biodistribution of Radio-Labelled DARPin Conjugates in hDLL3-MC38 Tumor Model

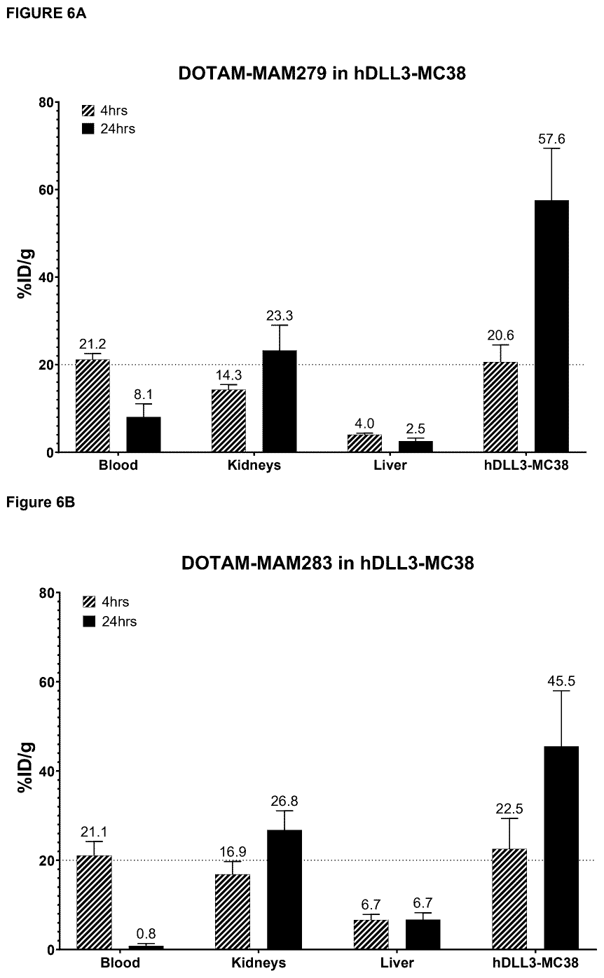

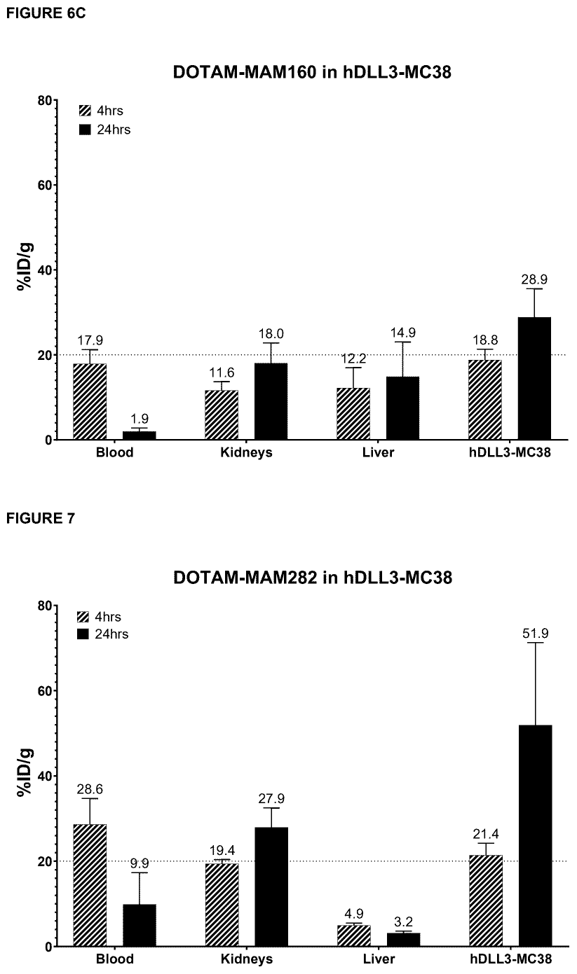

DARPins were conjugated with chelator (DOTAM) and labeled with Pb-212 as described in Example 3. Radio-labelled (Pb-212) DARPin conjugates were then injected at 0.01 mg/kg (10 μCi DARPin conjugates) into the tail vein of athymic nude mice (females) xenografted subcutaneously (at age of about 7 weeks) with hDLL3-MC38 cells (5 million cells, growth factor reduced Matrigel) and bearing tumors (5-10 mm in diameter). Biodistribution was monitored at 4 h and 24h post-injection. Mice were euthanized by CO2 inhalation and cervical dislocation. Blood, kidneys, liver, and tumor were extracted, weighed and the radioactivity was determined with a γ-counter (such as Wizard²2470, Perkin Elmer). The data were expressed as the % injected dose/gram (% ID/g). Results are shown in FIGS. 6A to 6C and Table 8.

TABLE 8

| | | |
|---|---|---|
| MAM279 | MAM283 | MAM160 |

| % ID/g | 4 h | 24 h | 4 h | 24 h | 4 h | 24 h |
|---|---|---|---|---|---|---|
| Blood | 21.2 | 8.1 | 21.1 | 0.8 | 17.9 | 1.9 |
| Kidneys | 14.3 | 23.3 | 16.9 | 26.8 | 11.6 | 18 |
| Tumors | 20.6 | 57.6 | 22.5 | 45.5 | 18.8 | 28.9 |
| T:K | 1.4 | 2.5 | 1.3 | 1.7 | 1.6 | 1.6 |
| ratio | | | | | | |

All radio-labelled DARPin (MAM279, MAM283, MAM160) conjugates tested in this example showed a T:K ratio above 1 at 4 and 24h in hDLL3-MC38 tumors (see Table 8).

### Example 8: Biodistribution of Radio-Labelled DARPin Conjugate in hDLL3-MC38 Tumor Model

DARPins were conjugated with chelator (DOTAM) and labeled with Pb-212 as described in Example 3. Radio-labelled (Pb-212) DARPin conjugate was then injected at 0.01 mg/kg (10 μCi DARPin conjugates) into the tail vein of athymic nude mice (females) xenografted subcutaneously (at age of about 7 weeks) with hDLL3-MC38 cells (5 million cells, growth factor reduced Matrigel) and bearing tumors (5-10 mm in diameter). Biodistribution was monitored at 4 h and 24h post-injection. Mice were euthanized by CO2 inhalation and cervical dislocation. Blood, kidneys, liver and tumor were extracted, weighed and the radioactivity was determined with a γ-counter (such as Wizard²2470, Perkin Elmer). The data were expressed as the % injected dose/gram (% ID/g). Results are shown in FIG. 7 and Table 9.

TABLE 9

MAM282

| % ID/g | 4 h | 24 h |
|---|---|---|
| Blood | 28.6 | 9.9 |
| Kidneys | 19.4 | 27.9 |
| Tumors | 21.4 | 51.9 |
| T:K ratio | 1.1 | 1.9 |

The radio-labelled DARPin (MAM282) conjugate tested in this example showed a T:K ratio above 1 at 4 and 24h in hDLL3-MC38 tumors (see Table 9).

### Example 9: Biodistribution of Radio-Labelled DARPin (MAM279) Conjugate in NCI-H82 and hDLL3-MC38 Tumor Models

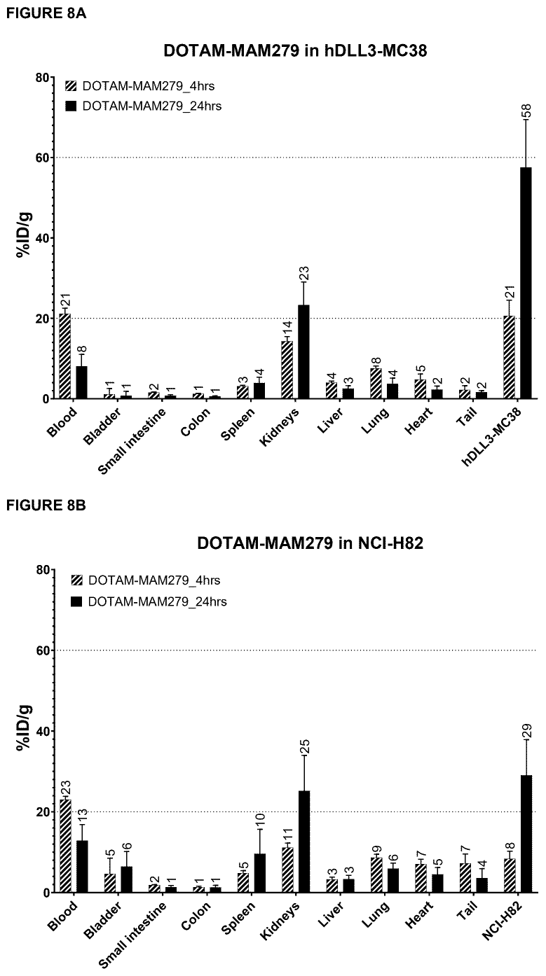

DARPins were conjugated with chelator (DOTAM) and labeled with Pb-212 as described in Example 3. Radio-labelled (Pb-212) DARPin conjugates were then injected at 0.01 mg/kg (10 μCi DARPin conjugates) (i) into the tail vein of R2G2 mice (females) xenografted subcutaneously (at age of about 7 weeks) with NCI-H82 cells (5 million cells, growth factor reduced Matrigel) and bearing tumors (5-10 mm in diameter), and (ii) into the tail vein of athymic nude mice (females) xenografted subcutaneously (at age of about 7 weeks) with hDLL3-MC38 cells (5 million cells, growth factor reduced Matrigel) and bearing tumors (5-10 mm in diameter). Biodistribution was monitored at 4 h and 24h post-injection. Mice were euthanized by CO2 inhalation and cervical dislocation. Blood, bladder, small intestine, colon, spleen, kidneys, liver, lung, heart, tail and tumor were extracted, weighed and the radioactivity was determined with a γ-counter (such as Wizard²2470, Perkin Elmer). The data were expressed as the % injected dose/gram (% ID/g). Results are shown in FIGS. 8A and 8B and Table 10.

TABLE 10

| | |
|---|---|
| MAM279 in | MAM279 in |
| hDLL3-MC38 | NCl-H82 |

| % ID/g | 4 h | 24 h | 4 h | 24 h |
|---|---|---|---|---|
| Blood | 21 | 8 | 23 | 13 |
| Kidneys | 14 | 23 | 11 | 25 |
| Tumors | 21 | 58 | 8 | 29 |
| T:K ratio | 1.5 | 2.5 | 0.8 | 1.2 |

The radio-labelled MAM279 conjugate showed a T:K ratio above 1 at 24h in the DLL3 low expression mouse model (NCI-H82) and in the DLL3 high expression mouse model (hDLL3-MC38) (see Table 10). A similar T:K ratio above 1 at 24h was obtained for the radio-labelled MAM279 conjugate in a DLL3 low expression mouse tumor model (NCI-H82), in which the NCI-H82 cells were xenografted into the R2G2 mice intravenously instead of subcutaneously (data not shown).

### Example 10: Tumor to Kidney Ratio of Radio-Labelled DARPin (MAM160, MAM282, MAM283) Conjugates in NCI-H82 Tumor Model

As described in Example 9 for radio-labelled MAM279 conjugate, the tumor to kidney (T:K) ratio in the NCI-H82 tumor model was also determined for other radio-labelled DARPin conjugates.

In brief, DARPins were conjugated with chelator (DOTAM) and labeled with Pb-212 as described in Example 3. Radio-labelled (Pb-212) DARPin (MAM160, MAM282, and MAM283) conjugates were then injected at 0.01 mg/kg (10 μCi DARPin conjugates) into the tail vein of R2G2 mice (females) xenografted subcutaneously (at age of about 7 weeks) with NCI-H82 cells (5 million cells, growth factor reduced Matrigel) and bearing tumors (5-10 mm in diameter). Biodistribution was monitored at 4 h and 24h post-injection. Mice were euthanized by CO2 inhalation and cervical dislocation. Different organs and tumor were extracted, weighed and the radioactivity was determined with a γ-counter (such as Wizard²2470, Perkin Elmer). The data were expressed as the % injected dose/gram (% ID/g). The tumor to kidney (T:K) ratio was determined as in Example 9. Results are shown in Table 13.

TABLE 13
Tumor to kidney ratio in NCI-H82 tumor model

| | | |
|---|---|---|
| MAM160 | MAM282 | MAM283 |

| | 4 h | 24 h | 4 h | 24 h | 4 h | 24 h |
|---|---|---|---|---|---|---|
| T:K | 0.6 | 1.0 | 0.6 | 1.0 | 0.7 | 1.1 |
| ratio | | | | | | |

The radio-labelled DARPin conjugates showed a T:K ratio of about 1 at 24h in the DLL3 low expression mouse model (NCI-H82) (see Table 13).

### Example 11: Target Binding Properties of Two Domain (2D) Ankyrin Repeat Protein MAM279 and of MAM279 Conjugated to a Chelator

Studies were performed to determine (i) whether 2D ankyrin repeat protein MAM279 binds to the N-terminal domain of human DLL3, (ii) whether MAM279 binds to DLL3 from different species, and (iii) whether the conjugation of a chelator of interest to MAM279 affects the target binding properties of the protein.

### Target Proteins

Several different target proteins were generated for these studies:

### Human Recombinant DLL3 Target Protein Preparation

Two target formats were chosen: i) the first target protein consisted of the extracellular domain of human DLL3 protein (Q9NYJ7, Gene ID: 10683, residues 27 to 466) linked via an Avi-tag (for site-directed biotinylation), followed by a 3C protease recognition site, to a Fc knob into hole (hDLL3-Avi-Fcₖᵢₕ); ii) the second target protein consisted of a very similar design including more residues of the extracellular domain of DLL3 (Q9NYJ7, Gene ID: 10683, res 27-470), directly linked to the Fc knob into hole domain followed by an Avi-tag (hDLL3-Fcₖᵢₕ-Avi).

hDLL3-Avi-Fcₖᵢₕ was expressed in CHO cells (Chinese hamster ovary cells) by Evitria and purified by a protein A derivative-based affinity chromatography followed by preparative size-exclusion chromatography (SEC). The material was up-concentrated in PBS pH 7.4 to 0.69 mg/ml and in vitro biotinylated using recombinant BirA. The final material was monomeric on size exclusion and stored in PBS pH 7.4 at the final concentration of 0.35 mg/ml.

hDLL3-Fcₖᵢₕ-Avi was co-expressed with BirA in Expi293F cells (human cells derived from the 293F cell line, Gibco) and purified by a protein A derivative-based affinity chromatography followed by preparative size-exclusion chromatography (SEC). The final material was monomeric on size exclusion and stored in PBS pH 7.4 at the final concentration of 0.43 mg/ml.

The truncated human DLL3 construct, comprising only the N-Terminal Domain (NTD, residues 27-189), was designed with a C-terminal 6×His and Avi-tag. Co-expression was carried in Expi293F cells and purification was performed via Ni-NTA affinity chromatography followed by SEC. The final material was monomeric on size exclusion and stored in PBS pH 7.4 at the final concentration of 0.26 mg/ml.

Recombinant DLL3 Target Protein Preparation from Other Species

The DLL3 target protein from cynomolgus macaque (cyno) was produced similarly to the human target protein. It consisted of the extracellular domain of the cyno DLL3 protein (XP_005589253.1, Gene ID:102115332, residues 27 to 466, purchased from Evitria) linked via an Avi-tag (for site-directed biotinylation), followed by a 3C protease recognition site, to a Fc knob into hole. The target protein was expressed in CHO cells (Chinese hamster ovary cells) by Evitria and purified by a protein A derivative-based affinity chromatography followed by preparative size exclusion chromatography (SEC). The material was up-concentrated in PBS pH 7.4 to 0.53 mg/ml and in vitro biotinylated using recombinant BirA. The final material was monomeric on size exclusion and stored in PBS pH 7.4 at the final concentration of 0.26 mg/ml.

The truncated dog DLL3 construct, comprising only the N-Terminal Domain (NTD, residues 27-189), was designed similarly to the human truncated target protein, with a C-terminal 6×His and Avi-tag. Co-expression was carried in Expi293F cells and purification was performed via Ni-NTA affinity chromatography followed by SEC. The final material was monomeric on size exclusion and stored in PBS pH 7.4 at the final concentration of ˜0.1-0.2 mg/ml.

### Experimental Description

The binding of 2D ankyrin repeat protein MAM279 as well as of MAM279 conjugated to the chelator DOTAM (the conjugation was performed as described in Example 3) to different target proteins was analyzed by Surface Plasmon Resonance (SPR). Specifically, the proteins analyzed were (i) MAM279 (SEQ ID NO: 11), with a Glycine-Serine (GS) at its N-terminal end and an N-terminal His-tag (SEQ ID NO: 19); and (ii) MAM279 (SEQ ID NO: 11), with a Glycine-Serine (GS) at its N-terminal end and a tag (SEQ ID NO: 10) at its C-terminal end (resulting in GS-MAM279-tag (SEQ ID NO: 15)), conjugated to the chelator DOTAM as described in Example 3.

A surface plasmon resonance (SPR) assay was used to determine the binding kinetics of the two proteins (i.e. MAM279 with and without conjugation to chelator) to different targets: the Extra-Cellular Domain (ECD) of human DLL3, the ECD of cynomolgus monkey DLL3, the N-Terminal Domain (NTD) only of human DLL3, and the NTD dog DLL3. The data were generated using BioRad ProteOn instrument in SPR running buffer (PBS, pH 7.4 containing 0.005% Tween 20©). A SAHC200M chip (Xantec) was conditioned according to the manufacturer's protocol. The chip was coated with biotinylated target to reach a signal intensity of 1516 RU (human ECD), 257 RU (human NTD), 1500 RU (cyno ECD) and 228 RU (dog NTD). All analytes (various concentrations in 3-fold dilutions ranging from 500-6.17 nM) were injected in succession for 240 s (100 μl/min) and dissociation was recorded for 10800 s (100 μl/min). Each injection was followed by a regeneration step with 16 mM H₃PO₄ for 18 s. The data was double referenced (control spot and buffer injection) and fitted to a 1:1 Langmuir model. The obtained SPR traces were used to determine on-rate and off-rate interaction parameters.

### Results

The results are summarized in Tables 14 and 15.

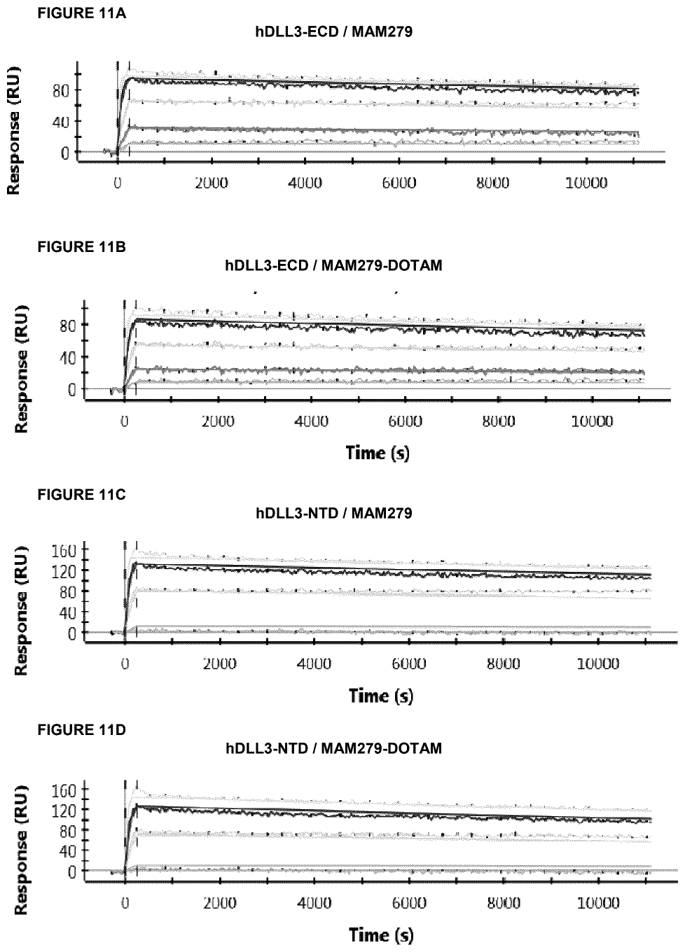

Representative SPR sensograms are shown in FIGS. 11A to 11D, for binding of MAM279 to hDLL3-ECD (FIG. 11A), binding of DOTAM-conjugated MAM279 to hDLL3-ECD (FIG. 11B), binding of MAM279 to hDLL3-NTD (FIG. 11C), and binding of DOTAM-conjugated MAM279 to hDLL3-NTD (FIG. 11D).

TABLE 14

| | Human DLL3-ECD | | | Human DLL3-NTD | | |
|---|---|---|---|---|---|---|
| DARPin | Kₒₙ [1/Ms] | K_{off} [1/s] | K_{D} [M] | Kₒₙ [1/Ms] | K_{off} [1/s] | K_{D} [M] |
| MAM279 | 8.35E+04 | 1.45E−05 | 1.74E−10 | 5.87E+04 | 1.54E−05 | 2.63E−10 |
| MAM279-DOTAM | 6.74E+04 | 1.64E−05 | 2.43E−10 | 5.04E+04 | 1.99E−05 | 3.94E−10 |

The results summarized in Table 14 demonstrated that the 2D ankyrin repeat protein MAM279 binds to the extracellular domain of human DLL3, and more specifically to the N-terminal domain of human DLL3, with a dissociation constant (K_{D}) in the picomolar range, and that the conjugation of a chelator of interest, such as DOTAM, to MAM279 via a C-terminal tag and a connector does not significantly affect the binding of MAM279 to human DLL3.

TABLE 15

| | Human DLL3-ECD | | | Cyno DLL3-ECD | | | Dog DLL3-NTD | | |
|---|---|---|---|---|---|---|---|---|---|
| | Kₒₙ | K_{off} | K_{D} | Kₒₙ | K_{off} | K_{D} | Kₒₙ | K_{off} | K_{D} |
| DARPin | [1/Ms] | [1/s] | [M] | [1/Ms] | [1/s] | [M] | [1/Ms] | [1/s] | [M] |
| MAM279 | 8.35E+04 | 1.45E−05 | 1.74E−10 | 4.45E+04 | 9.78E−04 | 2.20E−08 | 4.64E+04 | 5.58E−05 | 1.20E−09 |
| MAM279-DOTAM | 6.74E+04 | 1.64E−05 | 2.43E−10 | 3.60E+04 | 1.25E−03 | 3.46E−08 | 3.34E+04 | 4.86E−05 | 1.46E−09 |

The results summarized in Table 15 demonstrated that the 2D ankyrin repeat protein MAM279 binds to the extracellular domain of human DLL3 with a dissociation constant (K_{D}) in the picomolar range (1.74E-10 M), while MAM279 binds to DLL3 from cynomolgus monkey with a dissociation constant (K_{D}) that is about 100-fold higher (2.20E-08 M). Furthermore, the results demonstrated that 2D ankyrin repeat protein MAM279 binds to the extracellular domain of dog DLL3, more specifically to the N-terminal domain of dog DLL3, with a dissociation constant (K_{D}) of about 1 nanomole (1.20E-09 M). In other words, the 2D ankyrin repeat protein MAM279 binds to the extracellular domain of human DLL3 with a high affinity in the picomolar range, while it binds to the extracellular domain of cynomolgus monkey DLL3 with an about 100-fold lower affinity and to the N-terminal domain of dog DLL3 with an about 10-fold lower affinity.

The conjugation of a chelator of interest, such as DOTAM, to MAM279 via a C-terminal tag and a connector did not significantly affect the binding of MAM279 to human DLL3, cynomolgus monkey DLL3 or dog DLL3.

### Example 12: Thermal Stability Assessment of 2D DARPins Constructs

Labelling of proteins conjugated to a chelator with a radionuclide may require harsh conditions, including high temperatures. The thermal stability and the propensity to unfold/refold of 2D DARPins MAM279, MAM283 and MAM282 were assessed using Circular Dichroism (CD) spectroscopy.

The investigated DARPins included a Cysteine-containing tag at their C-terminal end, and hence they corresponded to SEQ ID NOs: 15, 16 and 18, respectively. Samples of the DARPins were diluted to 2 μM in PBS, pH7.4. The assessment was done using a Jasco J-815 spectrophotometer. The Tm (melting temperature) of the selected proteins was determined by CD as a parameter for thermal stability. In brief, the ellipticity was recorded at 222 nM and a temperature range from 20° C. to 90° C. was applied followed by reverse scan to record the refolding behavior. The Tm of the selected ankyrin repeat protein constructs is the midpoint of the protein unfolding. Spectra from 190-250 nm were recorded before and after the temperature scan.

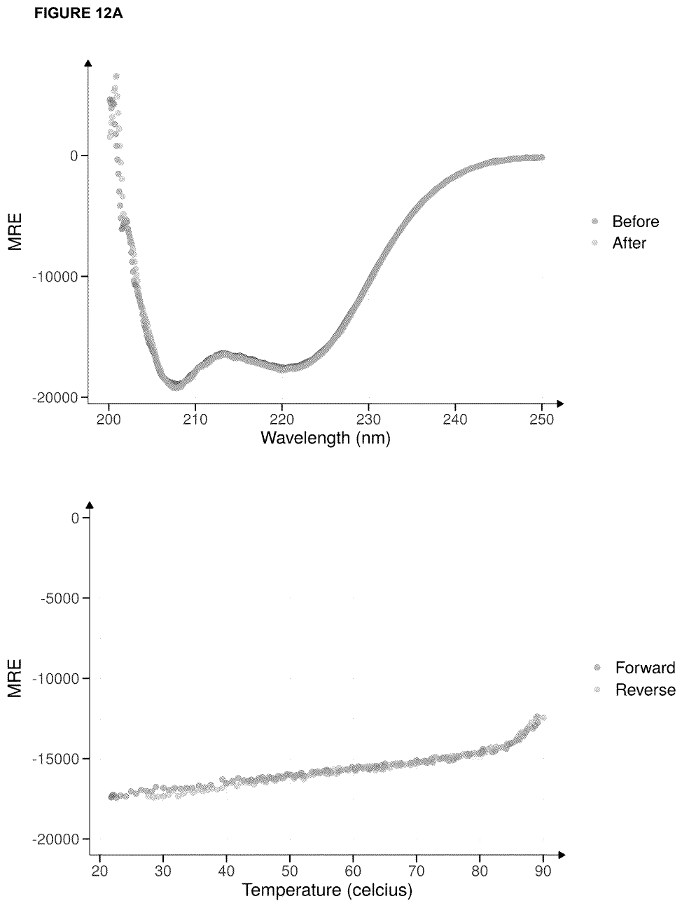

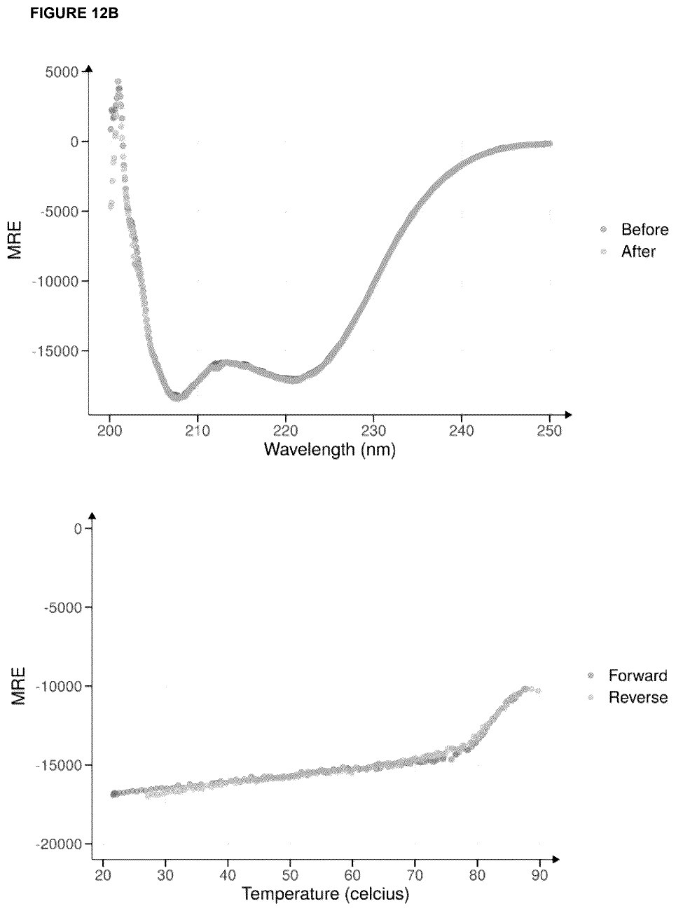

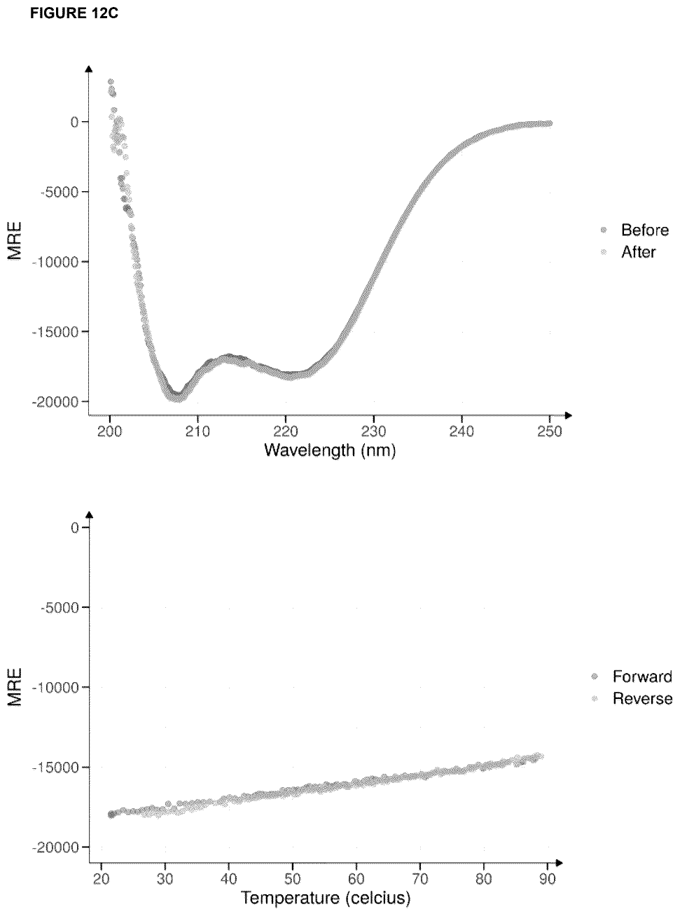

Results are shown in FIGS. 12A to 12C. The experiments demonstrated that the investigated DARPins have a high thermal stability. The melting temperatures (Tm) of the DARPins were determined to be above 85° C. for MAM279, about 82° C. for MAM283, and above 90° C. for MAM282.

### Example 13: Pharmacokinetic (PK) Analysis of Lead-Labelled DARPin (MAM279, MAM283, MAM160, MAM282) Conjugates in Mice

The pharmacokinetic (PK) properties of lead-labelled DARPin (MAM160, MAM279, MAM282, MAM283) conjugates were investigated in mice, including to determine the terminal half-life (or serum half-life) of these conjugates.

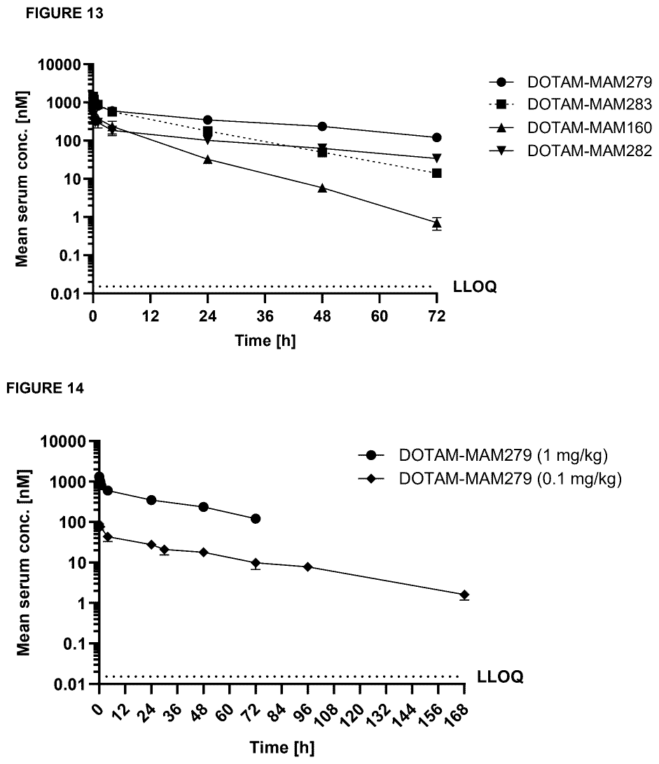

In brief, DARPins MAM279, MAM283, MAM160 and MAM282 were conjugated to the chelator DOTAM, via a C-terminal Cysteine containing tag and a connector, as described in Example 3. The conjugates were then labelled with natural lead (Pb). The protein components of the conjugates corresponded to SEQ ID NOs: 15 to 18, respectively. These lead-labelled DARPin conjugates were injected i.v. at 1 mg/kg into WT BALBc mice. Serum was collected 2 min, 10 min, 30 min, 1 h, 4 h, 24 h, 48 h, and 72 h post-injection. DARPins were detected and measured by ELISA. The data were expressed as the DARPin concentration in serum (nmol/L). Results are shown in FIG. 13 and Table 16.

TABLE 16

| Parameter | Unit | MAM279 | MAM283 | MAM160 | MAM282 |
|---|---|---|---|---|---|
| AUCINF_pred | (hr*nmol/L) | 29383.7 | 14494.3 | 4587.3 | 8435.0 |
| AUClast | (hr*hr*nmol/L) | 23892.0 | 14243.6 | 4578.7 | 7038.9 |
| Cmax | (nmol/L) | 1332.8 | 1435.2 | 625.1 | 460.6 |
| Tmax | (hr) | 0.033 | 0.033 | 0.033 | 0.033 |
| CI_pred | (L/hr/kg) | 0.0012 | 0.0023 | 0.0074 | 0.0040 |
| Vss_pred | (L/kg) | 0.048 | 0.035 | 0.060 | 0.153 |
| HL_Lambda_z | (hr) | 30.5 | 12.7 | 8.3 | 28.5 |
| AUC_% Extrap_pred | (%) | 19 | 2 | 0 | 17 |
| AUC_% Back_Ext_pred | (%) | 0 | 0 | 1 | 0 |

The results showed that among all the lead-labelled DARPin conjugates tested, the MAM279 conjugate has the longest serum half-life, with 30.5 hours. The MAM282 conjugate had a half-life of 28.5 hours, the MAM283 conjugate had a half-life of 12.7 hours and the MAM160 conjugate had a half-life of 8.3 hours (see Table 16 above).

### Example 14: Pharmacokinetic Analysis of Lead-Labelled DARPin (MAM279) Conjugate at Different Doses in Mice

The pharmacokinetic (PK) properties of lead-labelled DARPin (MAM279) conjugate were investigated in mice at different doses.

In brief, lead-labelled MAM279 conjugate, as described in Example 13, was injected i. at 0.1 mg/kg or at 1 mg/kg into the tail vein of WT BALBc mice. Serum was collected 2 min, 10 min, 30 min, 1 h, 4 h, 24 h, 48 h, and 72 h post-injection for MAM279 conjugate injected at 1 mg/kg, and 2 min, 30 min, 4 h, 24 h, 30 h, 48 h, 72 h, 96 h, and 168 h post-injection for MAM279 conjugate injected at 0.1 mg/kg. DARPins were detected and measured by ELISA. The data were expressed as the DARPin concentration in serum (nmol/L). Results are shown in FIG. 14 and Table 17.

TABLE 17

| | | MAM279 at | MAM279 at |
|---|---|---|---|
| Parameter | Unit | 1 mg/kg | 0.1 mg/kg |
| AUCINF_pred | (hr*nmol/L) | 29383.7 | 2405.1 |
| AUClast | (hr*hr*nmol/L) | 23892.0 | 2343.4 |
| Cmax | (nmol/L) | 1332.8 | 88.3 |
| Tmax | (hr) | 0.033 | 0.033 |
| CI_pred | (L/hr/kg) | 0.0012 | 0.0014 |
| Vss_pred | (L/kg) | 0.048 | 0.066 |
| HL_Lambda_z | (hr) | 30.5 | 32.8 |
| AUC_% Extrap_pred | (%) | 19 | 3 |
| AUC_% Back_Ext_pred | (%) | 0 | 0 |

The results showed that the lead-labelled MAM279 conjugate has a similar serum half-life, when injected at different doses. The MAM279 conjugate had a half-life of 30.5 hours when injected at 1 mg/kg, and a half-life of 32.8 hours when injected at 0.1 mg/kg (see Table 17 above).

### Example 15: Dose Response Finding Profile of Radio-Labelled DARPin (MAM279, MAM283) Conjugates in CD1 Mice

To determine the dose range of radio-labelled (Pb-212) DARPin (MAM279, MAM283) conjugates that can be tolerated by mice, different doses of the conjugates were evaluated for potential toxicity in mice.

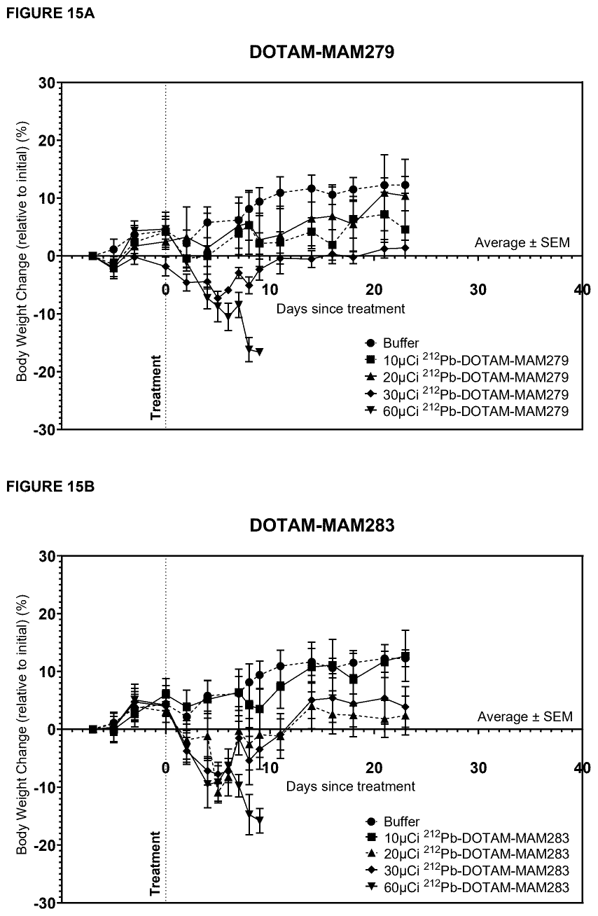

In brief, DARPins were conjugated with chelator (DOTAM) and labeled with Pb-212 as described in Example 3. Radio-labelled (Pb-212) DARPin (MAM279 and MAM283) conjugates (and buffer only as control) were then injected once at doses of 10 μCi, 20 μCi, 30 μCi and 60 μCi into the tail vein of WT CD1 mice (about 7 weeks old females). Animals were under observation daily and 3× per week animals were weighed. Animals were sacrificed 23 days after the first injection or if termination criteria were met (15% weight loss over 2 days or 20% from initial weight, lack of grooming over 5 days, lethargy/weakness over 3 days, reduced motility, hunched back, diarrhea, hypothermia, or the combination of multiple criteria). The data were expressed as % of body weight (BW) change (relative to the BW seven days before the treatment, i.e. the initial BW at day −7) (average+/−SEM). Results are shown in FIGS. 15A and 15B.

For both radio-labelled conjugates tested (MAM279 conjugate and MAM283 conjugate), animals appeared healthy and active at the injected doses of 10 μCi, 20 μCi and 30 μCi until they were sacrificed. Minimal weight loss occurred after injection of 10 μCi, 20 μCi and 30 μCi doses, which was rapidly recovered within up to about 10 days. All animals gained weight at doses up to 30 μCi. Furthermore, analysis of the hematology profile of the mice, assessing white blood cells, lymphocytes, monocytes, and neutrophils, demonstrated complete recovery within up to 28 days after injection of 10 μCi, 20 μCi and 30 μCi doses (data not shown). Toxicity was observed at the injected dose of 60 μCi.

### Example 16: Efficacy of Radio-Labelled DARPin (MAM279) Conjugate in NCI-H82 Tumor Model

To determine the efficacy of radio-labelled (Pb-212) DARPin (MAM279) conjugate in inhibiting the growth of tumors that express DLL3, different dose regimens (single or repeated dose) of the conjugate were tested in a relevant mouse tumor model (NCI-H82).

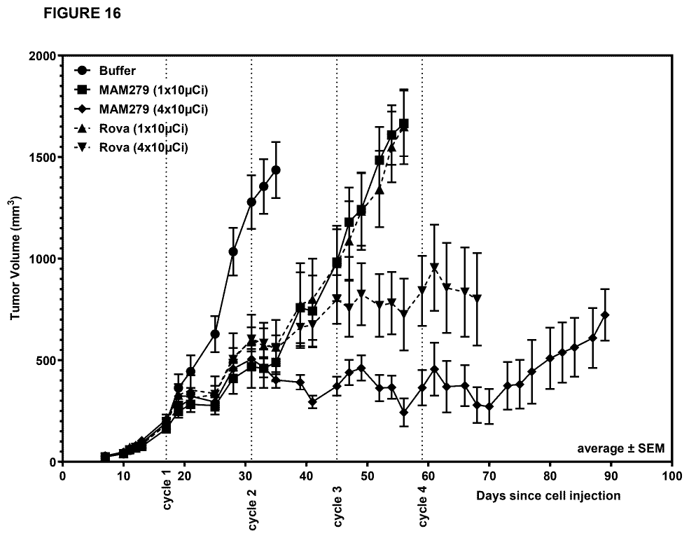

In brief, DARPins (or Rova antibody) were conjugated with chelator (DOTAM) and labeled with Pb-212 as described in Example 3. Radio-labelled (Pb-212) DARPin (MAM279) conjugate and Rova conjugate (used as control molecule) were injected at 1×10 μCi or at 4×10 μCi (injections 2 weeks apart) into the tail vein of R2G2 mice (females) xenografted subcutaneously (at age of about 7 weeks) with NCI-H82 cells, as indicated in FIG. 16. Animals were under observation daily and 3× per week tumors were measured by caliper. Animals were sacrificed when tumors reached 1500 to 2000 mm³ or if termination criteria were met. The data were expressed as average+/−SEM of tumor volume in mm³. Results are shown in FIG. 16 and Table 18.

TABLE 18

| Dunnett's multiple | Mean | 95.00% | Below | Sum- | Adjusted |
|---|---|---|---|---|---|
| comparisons test | Diff. | CI of diff. | threshold? | mary | P Value |
| Buffer vs. MAM279 | 140.6 | 38.19 to | Yes | ** | 0.0037 |
| conjugate | | 243.0 | | | |
| (1 × 10μCi) | | | | | |
| Buffer vs. MAM279 | 429.7 | 337.4 to | Yes | **** | <0.0001 |
| conjugate | | 522.1 | | | |
| (4 × 10μCi) | | | | | |
| Buffer vs. Rova | 119.6 | 17.18 to | Yes | * | 0.0171 |
| conjugate | | 222.0 | | | |
| (1 × 10μCi) | | | | | |
| Buffer vs. Rova | 256.3 | 163.9 to | Yes | **** | <0.0001 |
| conjugate | | 348.6 | | | |
| (4 × 10μCi) | | | | | |

The radio-labelled MAM279 conjugate showed good efficacy in inhibiting the growth of NCI-H82 tumors (see FIG. 16). Radio-labelled MAM279 conjugate at 4×10 μCi was more potent than the benchmark antibody (Rova) conjugate at 4×10 μCi. Statistical analysis done by Dunnett's multiple comparisons test showed that the radio-labelled MAM279 conjugate at 1×10 μCi or at 4×10 μCi had a significant inhibitory effect on the growth of NCI-H82 tumors (see Table 18). The tumor growth inhibition was more sustained with the repeat dosing regimen as compared to a single dose (see FIG. 16).

### Example 17: Binding of MAM279 and MAM093 to the Extracellular Domain of DLL3 from Different Species

Kinetic binding parameters and binding affinities of two domain (2D) ankyrin repeat protein MAM279 and single domain (1 D) ankyrin repeat protein MAM093 to DLL3 from different species were determined. The DLL3-specific ankyrin repeat domain of MAM093 is identical to the DLL3-specific ankyrin repeat domain comprised in MAM279.

SPR multi-trace analyses were performed similarly as described in Examples 1 and 11. Here, the binding kinetics of immobilized DARPin proteins to the extracellular domain of DLL3 (DLL3-ECD) from human, cynomolgus monkey, mouse and pig were determined. Various concentrations of recombinant DLL3-ECD starting from 500 nM were applied to immobilized biotinylated 2D DARPin (MAM279; SEQ ID NO: 11) or 1 D DARPin (MAM093; SEQ ID NO: 1) (both with a Glycine-Serine (GS) at the N-terminal end) for on-rate and off-rate measurements.

The data were generated using a ProteOn XPR36 instrument (BioRad) with SPR running buffer (PBS, pH 7.4 containing 0.005% Tween 20®). Streptavidin SAHC200M (Xantec) SPR chip was conditioned according to the manufacturer's protocol. Biotinylated DARPins were immobilized to a coating density of 27 resonance units (RU) for 1D DARPin and 25 RU for 2D DARPin. All analytes (3-fold dilution ranging from 500-6.17 nM) were injected in succession for 240 s and dissociation was recorded for 10800 s using a constant flow of 100 μl/min. The signal was double referenced (interspot and buffer injection) and fitted to a 1:1 Langmuir model. The obtained SPR traces were used to determine the DARPin-DLL3 interaction parameters, and results are summarized in Tables 19 and 20.

TABLE 19

| Biotinylated | | | | | | |
|---|---|---|---|---|---|---|
| DARPin | Human DLL3-ECD | | | Cyno DLL3-ECD | | |
| name | Kₒₙ [1/Ms] | K_{off} [1/s] | K_{D} [M] | Kₒₙ [1/Ms] | K_{off} [1/s] | K_{D} [M] |
| MAM279 | 1.02E+05 | 1.31E−05 | 1.27E−10 | 1.06E+05 | 9.39E−04 | 8.88E−09 |
| MAM093 | 9.07E+04 | 1.18E−05 | 1.30E−10 | 9.47E+04 | 8.25E−04 | 8.72E−09 |

TABLE 20

| Biotinylated | | | | | | |
|---|---|---|---|---|---|---|
| DARPin | mouse DLL3-ECD | | | pig DLL3-ECD | | |
| name | Kₒₙ [1/Ms] | K_{off} [1/s] | K_{D} [M] | Kₒₙ [1/Ms] | K_{off} [1/s] | K_{D} [M] |
| MAM279 | 3.37E+04 | 2.82E−03 | 8.39E−08 | 1.84E+04 | 3.19E−04 | 1.73E−08 |
| MAM093 | 2.67E+04 | 4.00E−03 | 1.49E−07 | 2.31E+04 | 3.30E−04 | 1.43E−08 |

The results showed that MAM093 and MAM279 specifically bind to the extracellular domain of DLL3 from various mammalian species (human, cyno, mouse, pig), but with different affinities. MAM093 and MAM279 bind with the highest affinity (in the pM range) to human DLL3, while the binding affinities to DLL3 from the other species are lower (in the nM range). The results also demonstrated that the addition of an ankyrin repeat domain with binding specificity for serum albumin (connected by a peptide linker) does not significantly affect the binding kinetics and affinity of the DLL3-specific ankyrin repeat domain (MAM093) to its target.

### Example 18: Binding Properties of Two Domain (2D) Ankyrin Repeat Protein MAM279 and of MAM279 Conjugated to a Chelator to Serum Albumin

Studies were performed to determine (i) kinetic binding parameters and binding affinities of two domain (2D) ankyrin repeat protein MAM279 to human and mouse serum albumin, and (ii) whether the conjugation of a chelator of interest to MAM279 affects the binding properties of the protein to serum albumin.

SPR multi-trace analyses were performed similarly as described in previous Examples. The following ankyrin repeat proteins analyzed: (i) MAM279 (SEQ ID NO: 11) (with a Glycine-Serine (GS) at its N-terminal end); and (ii) MAM279 (SEQ ID NO: 11), with a Glycine-Serine (GS) at its N-terminal end and a tag (SEQ ID NO: 10) at its C-terminal end (resulting in GS-MAM279-tag (SEQ ID NO: 15)), conjugated to the chelator DOTAM as described in Example 3. The unconjugated MAM279 had an N-terminal His-tag (SEQ ID NO: 19) for ease of purification. Various concentrations of these ankyrin repeat proteins starting from 600 nM were applied to immobilized human serum albumin (HSA) or mouse serum albumin (MSA) for on-rate and off-rate measurements.

The data were generated using a ProteOn XPR36 instrument (BioRad) with SPR running buffer (PBS, pH 7.4 containing 0.005% Tween 20®). A HC200M chip (Xantec) was conditioned according to the manufacturer's protocol. The chip was coated with purified human serum albumin (HSA, CSL) or mouse serum albumin (MSA, Sigma-Aldrich) in NaOAc pH5.0 to reach a signal intensity of ˜447 (HSA) or ˜644 (MSA) RU. All analytes (3-fold dilution ranging from 600-7.41 nM) were injected in succession for 240 s and dissociation was recorded for 600 s using a constant flow of 25 μl/min (HSA) or 100 μl/min (MSA) (25 μl/min). The signal was double referenced (interspot and buffer injection) and fitted to a 1:1 Langmuir model. The obtained SPR traces were used to determine the DARPin-serum albumin interaction parameters, and results are summarized in Table 21.

TABLE 21

| DARPin | HSA | | | MSA | | |
|---|---|---|---|---|---|---|
| name | Kₒₙ [1/Ms] | K_{off} [1/s] | K_{D} [M] | Kₒₙ [1/Ms] | K_{off} [1/s] | K_{D} [M] |
| MAM279 | 2.86E+05 | 2.16E−02 | 7.55E−08 | 2.67E+05 | 3.83E−02 | 1.44E−07 |
| MAM279- | 2.13E+05 | 2.10E−02 | 19.82E−08 | 1.55E+05 | 4.25E−02 | 2.74E−07 |
| DOTAM | | | | | | |

The results demonstrated that the 2D ankyrin repeat protein MAM279 specifically binds to human serum albumin and mouse serum albumin, with dissociation constants (K_{D}) in the nanomolar range, and that the conjugation of a chelator of interest, such as DOTAM, to MAM279 via a C-terminal tag and a connector does not significantly affect the binding of MAM279 to human or mouse serum albumin.

### Example 19: Simultaneous Binding of a Two Domain (2D) DARPin (MAM279) Conjugate to DLL3 and Serum Albumin

Studies were performed to determine whether a two domain (2D) ankyrin repeat protein conjugate, as described herein, can bind simultaneously to DLL3 and serum albumin. The specific conjugate tested was MAM279 (SEQ ID NO: 11), with a Glycine-Serine (GS) at its N-terminal end and a tag (SEQ ID NO: 10) at its C-terminal end (resulting in GS-MAM279-tag (SEQ ID NO: 15)), conjugated to the chelator DOTAM as described in Example 3.

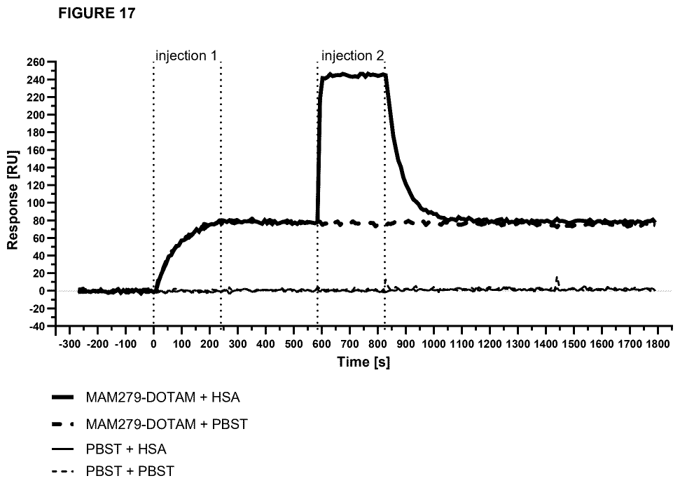

An SPR assay was used to assess the concurrent binding of DARPin (MAM279) conjugate to both human DLL3 and human serum albumin (HSA). The data were generated using a ProteOn XPR36 instrument (BioRad) with SPR running buffer (PBS, pH 7.4 containing 0.005% Tween 20®). A streptavidin SAHC200M (Xantec) sensor chip was conditioned according to the manufacturer's protocol. Biotinylated recombinant human DLL3-ECD was immobilized to a coating density of 1350-1470 RU. A single injection of 150 nM DARPin conjugate was performed, with an association time of 240 s using a constant flow of 100 μl/min. After a lag time of ˜300 seconds, HSA was injected at 1000 nM with an association time of 240 seconds and a dissociation time of 900 seconds using a constant flow of 100 μl/min. Control lanes were used where only one of the analytes or only PBST was injected. The signal was double referenced (interspot and buffer injection). The figure (FIG. 17) was prepared with GraphPad Prism 10.2.3.

This study demonstrated that a two domain (2D) ankyrin repeat protein conjugate, as described herein, can bind simultaneously to DLL3 and serum albumin. Specifically, the results showed that MAM279-DOTAM conjugate can bind simultaneously to human DLL3 and human serum albumin (see FIG. 17).

### Example 20: Internalization of Half-Life Extended DLL3-Specific Ankyrin Repeat Protein (MAM279) in Cells Expressing DLL3 on their Surface

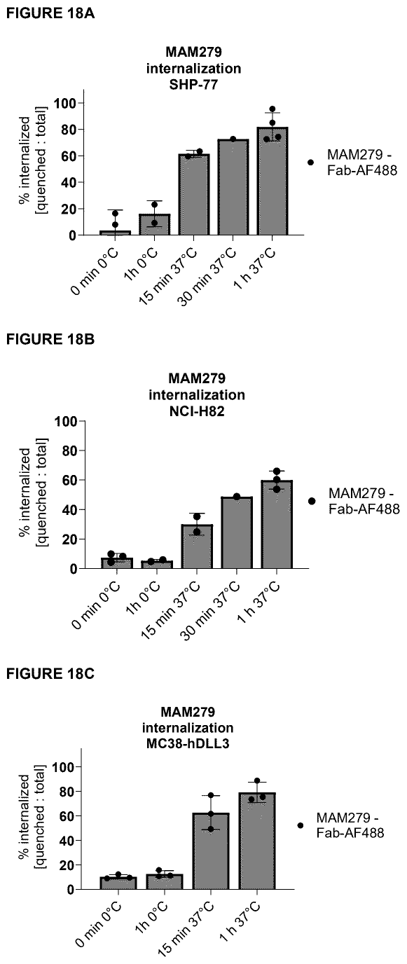

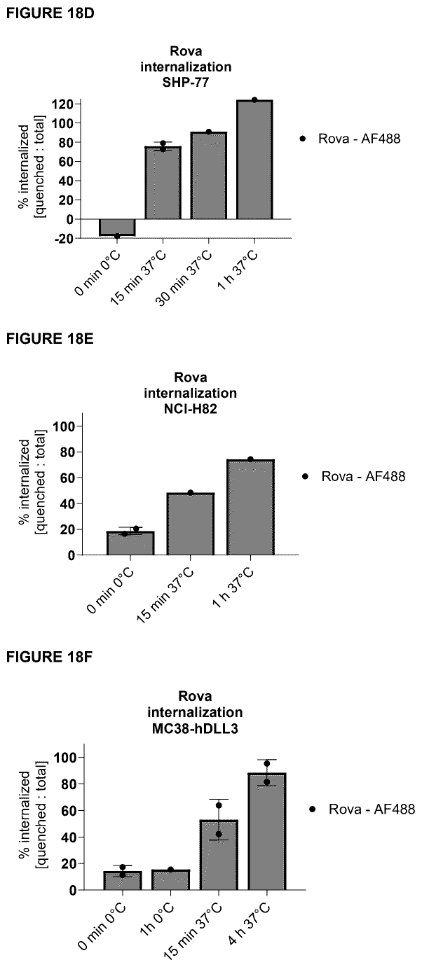

Studies were performed to determine whether a half-life extended DLL3-specific ankyrin repeat protein, as described herein, is internalized upon binding to cells expressing DLL3 on their surface. The specific ankyrin repeat protein tested was MAM279 (SEQ ID NO: 11) (with a Glycine-Serine (GS) at its N-terminal end). The protein had an N-terminal His-tag (SEQ ID NO: 19) for ease of purification. As a comparison, internalization was also tested for Rova antibody. Internalization was investigated on different cell types expressing DLL3 on their surface, namely SHP-77 lung carcinoma cells, NCI-H82 lung carcinoma cells, and hDLL3-expressing MC38 (MC38-hDLL3) colon carcinoma cells. In brief, DARPin and antibody were pre-incubated and labelled with fluorescently labelled detection antibodies (Fab-AF488) and added to 5×10⁴ cells/well in a 96-well plate (100 nM DARPin, 10 nM antibody control) and incubated for 1h on ice. Cells were washed once with PBS and then incubated for different timepoints (0 min, 15 min, 30 min, 1 h and/or 4 h) at 37° C. in PBS with 2% FCS. Anti-AF488 quencher (A11094, Invitrogen) and Live/Dead NIR (1:3000, Thermo Fisher #L34957) in PBS were added to the “quencher” wells, and Live/Dead NIR to the “non-quenched” wells, for 1h on ice, to quench the external (membrane bound) signal. After washing, cells were fixed (Paraformaldehyde Solution, LucernaChem) and acquired at Attune N×T (Thermo Life Technologies). Raw fcs files were exported and analyzed using FlowJo software. MFI values of live AF488-positive cells were exported from FlowJo and plotted using GraphPad Prism software. % internalization was determined by normalizing to non-binding control DARPin (for MAM279) or non-binding isotype control IgG (for Rova). None of the tested ankyrin repeat proteins or antibody showed binding to or internalization in MC38 wildtype cells (which do not express hDLL3 on their surface). Results are shown in FIGS. 18A to 18C for MAM279 DARPin and in FIGS. 18D to 18F for Rova antibody.

The results confirmed the internalization of Rova antibody in SHP-77 cells (FIG. 18D), NCI-H82 cells (FIG. 18E) and MC38-hDLL3 cells (FIG. 18F). Furthermore, the results demonstrated that also a half-life extended DLL3-specific ankyrin repeat protein (MAM279) is similarly internalized in cells expressing DLL3 on their surface, such as SHP-77 cells (FIG. 18A), NCI-H82 cells (FIG. 18B) and MC38-hDLL3 cells (FIG. 18C).

### Example 21: Pharmacokinetic Analysis of Lead-Labelled DARPin (MAM279) Conjugate at Different Doses in Mice

The pharmacokinetic (PK) properties of lead-labelled DARPin (MAM279) conjugate were investigated in mice at further doses (0.01 mg/kg and 0.001 mg/kg), in addition to the doses shown in Example 14 (1 mg/kg and 0.1 mg/kg). Furthermore, the PK properties of Rova antibody were tested as well, for a comparison with the DARPin conjugate. A summary of the results obtained with the various doses of lead-labelled DARPin (MAM279) conjugate and Rova antibody is described and shown in this Example.

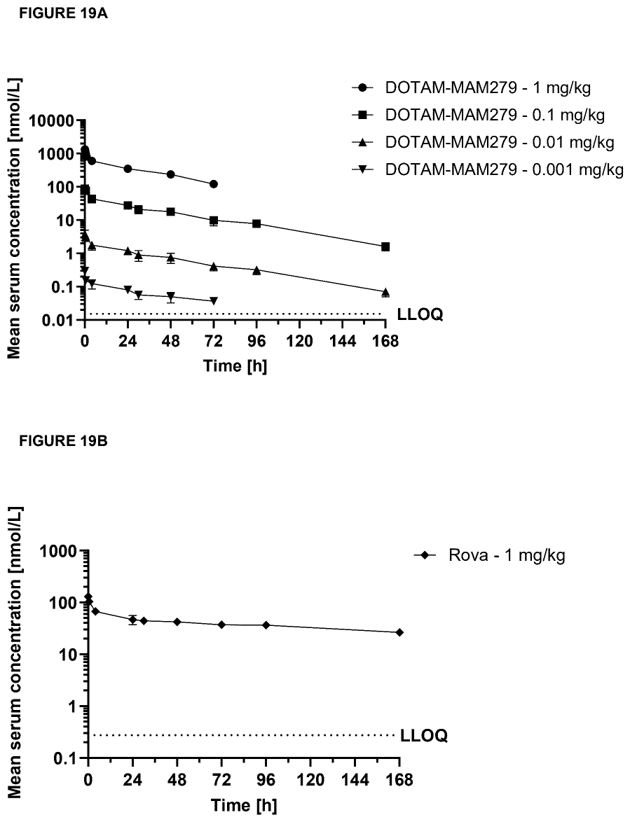

In brief, natural lead-labelled MAM279 conjugate, as described in Example 13, was injected i.v. at different doses (0.001 mg/kg, 0.01 mg/kg, 0.1 mg/kg, or 1 mg/kg) into the tail vein of WT BALBc mice. Similarly, unconjugated Rova was injected iv. at 1 mg/kg into the tail vein of WT BALBc mice. Serum was collected 2 min, 10 min, 30 min, 1 h, 4 h, 24 h, 48 h, and 72 h post-injection for MAM279 conjugate injected at 1 mg/kg, and 2 min, 30 min, 4 h, 24 h, 30 h, 48 h, 72 h, 96 h, and 168 h post-injection for MAM279 conjugate injected at 0.1 mg/kg, 0.01 mg/kg and 0.001 mg/kg. Serum was collected 2 min, 30 min, 4 h, 24 h, 30 h, 48 h, 72 h, 96 h, and 168 h post-injection for Rova antibody injected at 1 mg/kg. DARPins and Rova were detected and measured by ELISA. Rova PK characteristics were calculated with time points between 4 h and 168 h and MAM279 conjugate PK characteristics were calculated with time points between 4 h and 72 h. The data were expressed as the DARPin or Rova concentration in serum (nmol/L). Results are shown in FIGS. 19A and 19B and Table 22.

TABLE 22

| | | Values | | | | |
|---|---|---|---|---|---|---|
| | | Rova | MAM279 | MAM279 | MAM279 | MAM279 |
| Parameter | Unit | 1 mg/kg | 1 mg/kg | 0.1 mg/kg | 0.01 mg/kg | 0.001 mg/kg |
| AUCINF_pre | (h*nmol/L) | 11918.3 | 29383.7 | 2405.1 | 101.0 | 7.0 |
| AUClast | (h*hr*nmol/L) | 6636.1 | 23892.0 | 2343.4 | 98.3 | 5.0 |
| Cmax | (nmol/L) | 129.5 | 1332.8 | 88.3 | 3.7 | 0.29 |
| Tmax | (h) | 0.033 | 0.033 | 0.033 | 0.033 | 0.033 |
| Cl_pred | (L/h/kg) | 0.0006 | 0.0012 | 0.0014 | 0.0034 | 0.0049 |
| Vss_pred | (L/kg) | 0.12 | 0.048 | 0.066 | 0.157 | 0.267 |
| HL_Lambda_z | (h) | 145.8 | 30.5 | 32.8 | 33.1 | 39.9 |
| AUC_%Extrap_Pred | (%) | 44 | 19 | 3 | 3 | 28 |
| AUC_%Back_Ext_pre | (%) | 0 | 0 | 0 | 0 | 0 |

The results showed that the lead-labelled MAM279 conjugate displays a similar serum half-life, when injected at different doses within a large dose range. The MAM279 conjugate had a half-life of 30.5 hours when injected at 1 mg/kg, of 32.8 hours when injected at 0.1 mg/kg, of 33.1 hours when injected at 0.01 mg/kg, and of 39.9 hours when injected at 0.001 mg/kg (see Table 22 above). Thus, taken together, the MAM279 conjugate displayed a terminal half-life of about 30 to 40 hours in mice when injected at a dose range of 0.001 mg/kg to 1 mg/kg, or a terminal half-life of about 30 to 34 hours when injected at a dose range of 0.01 mg/kg to 1 mg/kg. The Rova antibody displayed a significantly longer half-life in mice than the MAM279 conjugate (e.g. of about 145 hours, when injected at 1 mg/kg and calculated with time points between 4 hours and 168 hours, see Table 22).

### Example 22: Efficacy of Weekly Dosing of Radio-Labelled DARPin (MAM279) Conjugate in NCI-H82 Tumor Model

To determine the efficacy of more frequent repeat-dosing of radio-labelled (Pb-212) DARPin (MAM279) conjugate in inhibiting the growth of tumors that express DLL3, a weekly repeat-dosing regimen of the conjugate was tested in a relevant mouse tumor model (NCI-H82).

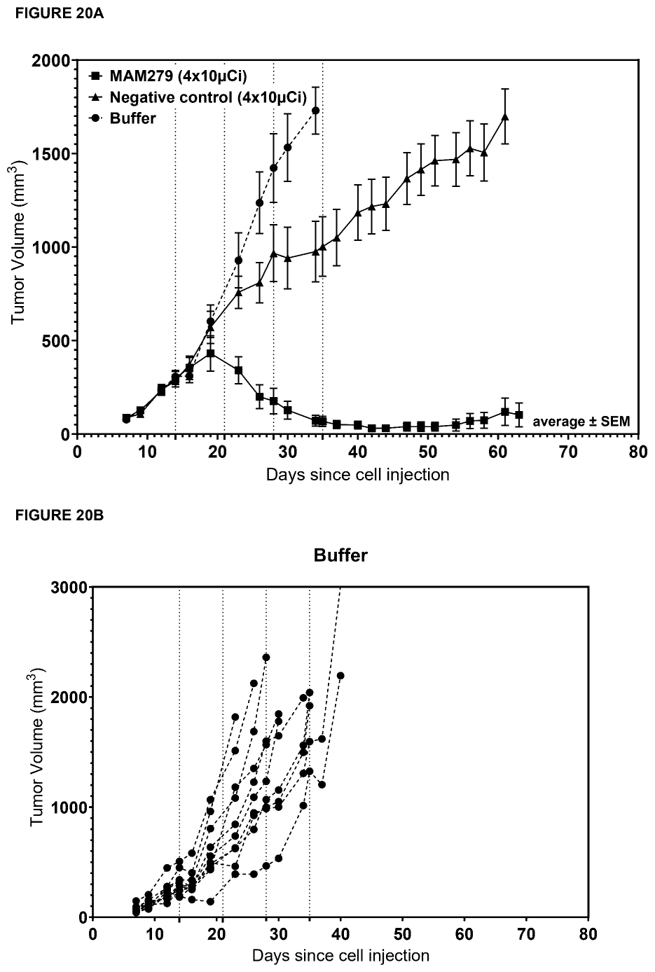

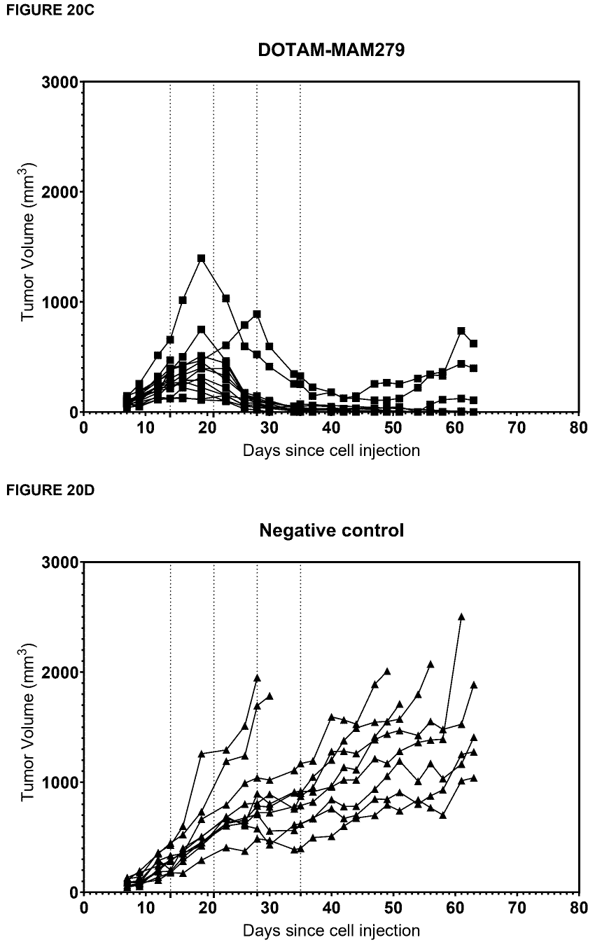

In brief, MAM279 (with a N-terminal GS and C-terminal Cysteine-containing tag) (SEQ ID NO: 15) or a negative control DARPin (SEQ ID NO: 26), in which the DLL3-specific ankyrin repeat domain of MAM279 was replaced with a non-binding ankyrin repeat domain (SEQ ID NO: 21), were conjugated with chelator (DOTAM) and labeled with Pb-212 as described in Example 3. Radio-labelled MAM279 conjugate and DARPin control conjugate were injected at 4×10 μCi (injections 1 week apart) into the tail vein of R2G2 mice (females) xenografted subcutaneously (at age of about 7 weeks) with NCI-H82 cells, as indicated in FIGS. 20A to 20D. Corresponding injections of buffer only were done as control. The first weekly injection of radio-labeled molecules was done 14 days after xenografting the NCI-H82 cells. Animals were under observation daily and 3× per week tumors were measured by caliper. Animals were sacrificed when tumors reached 1500 to 2000 mm³ or if termination criteria were met. The data were expressed as average+/−SEM of tumor volume in mm³. Results are shown in FIGS. 20A to 20D and Table 23.

TABLE 23

| Dunnett's multiple | Mean | 95.00% | Below | Sum- | Adjusted |
|---|---|---|---|---|---|
| comparisons test | Diff. | CI of diff. | threshold? | mary | P Value |
| Buffer only vs. | 731.9 | 621.2 to | Yes | **** | <0.0001 |
| DOTAM-MAM279 | | 842.6 | | | |
| (4 × 10μCi, 1 wk) | | | | | |
| Buffer only vs. | −64.44 | −180.1 to | No | ns | 0.3392 |
| neg. control | | 51.25 | | | |
| (4 × 10μCi, 1 wk) | | | | | |

The radio-labelled MAM279 conjugate (but not the negative control) showed strong and statistically significant efficacy in inhibiting the growth of NCI-H82 tumors (see FIGS. 20A to 20D and Table 23) when injected four times weekly at 10 μCi. The weekly repeat-dosing of radio-labelled MAM279 conjugate resulted in complete tumor regression in about 70% of mice at day 63 post-tumor cell xenograft.

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications, patents, and GenBank sequences cited in this disclosure are incorporated by reference in their entirety. To the extent the material incorporated by reference contradicts or is inconsistent with this specification, the specification will supersede any such material. The citation of any references herein is not an admission that such references are prior art to the present invention.

Those skilled in the art will recognize, or be able to ascertain using no more than routine experimentation, many equivalents to the specific embodiments of the invention described herein. Such equivalents are intended to be encompassed by the following claims.

SEQUENCE TABLE

| SEQ | | | |
|---|---|---|---|
| ID | Description | Alternative | |
| NO | of sequence | names | Sequence |
| 1 | Ankyrin | MAM093 | DLGKKLLAAAAQGQDDEVRELLKAGANVNAKD |
| | repeat | | LFGYTPLHLAAWRGHLEIVEVLLKAGADVNAKD |
| | domain | | AAGETPLHLAAWFGHLEIVEVLLKAGADVNAQD |
| | specific for | | LQGKTPADLAAKEGHEDIAEVLQKAA |
| | DLL3 | | |
| 2 | Ankyrin | MAM088 | DLGKKLLAAAREGQDDEVRELLKAGADVNAKDI |
| | repeat | | WGQTPLHIAAWFGHLEIVEVLLKAGADVNAKDQ |
| | domain | | TGHTPLHLAAKFGHLEIVEVLLKAGADVNAQDLL |
| | specific for | | GRTPADLAAEEGHEDIAEVLQKAA |
| | DLL3 | | |
| 3 | Ankyrin | MAM112 | DLGKKLLAAAADGQDDEVRELLQAGADVNAKD |
| | repeat | | LFGYTPLHLAAWRGHLEIVEVLLEAGADVNAKD |
| | domain | | AAGETPLHLAAWFGHLEIVEVLLEAGADVNAQD |
| | specific for | | VQGKTPADLAAKEGHEDIAEVLQQAA |
| | DLL3 | | |
| 4 | Ankyrin | MAM120 | DLGTKLLSAAADGQDDEVRELLQAGADVNAKD |
| | repeat | | LFGYTPLHLAAWRGHLEIVEVLLEAGADVNAKD |
| | domain | | AAGLTPLHLAAWFGHLEIVEVLLEAGADVNAQD |
| | specific for | | VQGVTPADLAAKEGHEDIAEVLQQAA |
| | DLL3 | | |
| 5 | Ankyrin | | DLGKKLLEAARAGQDDEVRELLKAGADVNAKD |
| | repeat | | YFSHTPLHLAARNGHLKIVEVLLKAGADVNAKD |
| | domain | | FAGKTPLHLAANEGHLEIVEVLLKAGADVNAQDI |
| | specific for | | FGKTPADIAADAGHEDIAEVLQKAA |
| | human serum | | |
| | albumin | | |
| 6 | Ankyrin | | DLGKKLLEAARAGQDDEVRELLKAGADVNAKD |
| | repeat | | YFSHTPLHLAARNGHLKIVEVLLKAGADVNAKD |
| | domain | | FAGKTPLHLAAADGHLEIVEVLLKAGADVNAQDI |
| | specific for | | FGKTPADIAADAGHEDIAEVLQKAA |
| | human serum | | |
| | albumin | | |
| 7 | Ankyrin | | DLGKKLLEAARAGQDDEVRELLKAGADVNAKD |
| | repeat | | YFSHTPLHLAARNGHLKIVEVLLKAGADVNAKD |
| | domain | | FAGKTPLHLAADAGHLEIVEVLLKAGADVNAQDI |
| | specific for | | FGKTPADIAADAGHEDIAEVLQKAA |
| | human serum | | |
| | albumin | | |
| 8 | PT linker | | GSPTPTPTTPTPTPTTPTPTPTGS |
| 9 | Consensus | | [GGGGS]n, wherein n is 1, 2, 3, 4, 5, or 6 |
| | GS linker | | |
| 10 | Tag | | GSGSC |
| 11 | Ankyrin | MAM279 | DLGKKLLEAARAGQDDEVRELLKAGADVNAKD |
| | repeat protein | | YFSHTPLHLAARNGHLKIVEVLLKAGADVNAKD |
| | | | FAGKTPLHLAADAGHLEIVEVLLKAGADVNAQD |
| | | | IFGKTPADIAADAGHEDIAEVLQKAAGSPTPTPT |
| | | | TPTPTPTTPTPTPTGSDLGKKLLAAAAQGQDDE |
| | | | VRELLKAGANVNAKDLFGYTPLHLAAWRGHLEI |
| | | | VEVLLKAGADVNAKDAAGETPLHLAAWFGHLEI |
| | | | VEVLLKAGADVNAQDLQGKTPADLAAKEGHED |
| | | | IAEVLQKAA |
| 12 | Ankyrin | MAM283 | DLGKKLLEAARAGQDDEVRELLKAGADVNAKD |
| | repeat protein | | YFSHTPLHLAARNGHLKIVEVLLKAGADVNAKD |
| | | | FAGKTPLHLAADAGHLEIVEVLLKAGADVNAQD |
| | | | IFGKTPADIAADAGHEDIAEVLQKAAGSPTPTPT |
| | | | TPTPTPTTPTPTPTGSDLGKKLLAAAADGQDDE |
| | | | VRELLQAGADVNAKDLFGYTPLHLAAWRGHLEI |
| | | | VEVLLEAGADVNAKDAAGETPLHLAAWFGHLEI |
| | | | VEVLLEAGADVNAQDVQGKTPADLAAKEGHED |
| | | | IAEVLQQAA |
| 13 | Ankyrin | MAM160 | DLGKKLLEAARAGQDDEVRELLKAGADVNAKD |
| | repeat protein | | YFSHTPLHLAARNGHLKIVEVLLKAGADVNAKD |
| | | | FAGKTPLHLAADAGHLEIVEVLLKAGADVNAQD |
| | | | IFGKTPADIAADAGHEDIAEVLQKAAGSPTPTPT |
| | | | TPTPTPTTPTPTPTGSDLGTKLLSAAADGQDDE |
| | | | VRELLQAGADVNAKDLFGYTPLHLAAWRGHLEI |
| | | | VEVLLEAGADVNAKDAAGLTPLHLAAWFGHLEI |
| | | | VEVLLEAGADVNAQDVQGVTPADLAAKEGHED |
| | | | IAEVLQQAA |
| 14 | Ankyrin | MAM282 | DLGKKLLEAARAGQDDEVRELLKAGADVNAKD |
| | repeat protein | | YFSHTPLHLAARNGHLKIVEVLLKAGADVNAKD |
| | | | FAGKTPLHLAADAGHLEIVEVLLKAGADVNAQD |
| | | | IFGKTPADIAADAGHEDIAEVLQKAAGSPTPTPT |
| | | | TPTPTPTTPTPTPTGSDLGKKLLAAAREGQDDE |
| | | | VRELLKAGADVNAKDIWGQTPLHIAAWFGHLEI |
| | | | VEVLLKAGADVNAKDQTGHTPLHLAAKFGHLEI |
| | | | VEVLLKAGADVNAQDLLGRTPADLAAEEGHEDI |
| | | | AEVLQKAA |
| 15 | Ankyrin | GS-MAM279- | GSDLGKKLLEAARAGQDDEVRELLKAGADVNA |
| | repeat protein | tag | KDYFSHTPLHLAARNGHLKIVEVLLKAGADVNA |
| | with tag | | KDFAGKTPLHLAADAGHLEIVEVLLKAGADVNA |
| | | | QDIFGKTPADIAADAGHEDIAEVLQKAAGSPTPT |
| | | | PTTPTPTPTTPTPTPTGSDLGKKLLAAAAQGQD |
| | | | DEVRELLKAGANVNAKDLFGYTPLHLAAWRGH |
| | | | LEIVEVLLKAGADVNAKDAAGETPLHLAAWFGH |
| | | | LEIVEVLLKAGADVNAQDLQGKTPADLAAKEGH |
| | | | EDIAEVLQKAAGSGSC |
| 16 | Ankyrin | GS-MAM283- | GSDLGKKLLEAARAGQDDEVRELLKAGADVNA |
| | repeat protein | tag | KDYFSHTPLHLAARNGHLKIVEVLLKAGADVNA |
| | with tag | | KDFAGKTPLHLAADAGHLEIVEVLLKAGADVNA |
| | | | QDIFGKTPADIAADAGHEDIAEVLQKAAGSPTPT |
| | | | PTTPTPTPTTPTPTPTGSDLGKKLLAAAADGQD |
| | | | DEVRELLQAGADVNAKDLFGYTPLHLAAWRGH |
| | | | LEIVEVLLEAGADVNAKDAAGETPLHLAAWFGH |
| | | | LEIVEVLLEAGADVNAQDVQGKTPADLAAKEGH |
| | | | EDIAEVLQQAAGSGSC |
| 17 | Ankyrin | GS-MAM160- | GSDLGKKLLEAARAGQDDEVRELLKAGADVNA |
| | repeat protein | tag | KDYFSHTPLHLAARNGHLKIVEVLLKAGADVNA |
| | with tag | | KDFAGKTPLHLAADAGHLEIVEVLLKAGADVNA |
| | | | QDIFGKTPADIAADAGHEDIAEVLQKAAGSPTPT |
| | | | PTTPTPTPTTPTPTPTGSDLGTKLLSAAADGQD |
| | | | DEVRELLQAGADVNAKDLFGYTPLHLAAWRGH |
| | | | LEIVEVLLEAGADVNAKDAAGLTPLHLAAWFGH |
| | | | LEIVEVLLEAGADVNAQDVQGVTPADLAAKEGH |
| | | | EDIAEVLQQAAGSGSC |
| 18 | Ankyrin | GS-MAM282- | GSDLGKKLLEAARAGQDDEVRELLKAGADVNA |
| | repeat protein | tag | KDYFSHTPLHLAARNGHLKIVEVLLKAGADVNA |
| | with tag | | KDFAGKTPLHLAADAGHLEIVEVLLKAGADVNA |
| | | | QDIFGKTPADIAADAGHEDIAEVLQKAAGSPTPT |
| | | | PTTPTPTPTTPTPTPTGSDLGKKLLAAAREGQD |
| | | | DEVRELLKAGADVNAKDIWGQTPLHIAAWFGHL |
| | | | EIVEVLLKAGADVNAKDQTGHTPLHLAAKFGHL |
| | | | EIVEVLLKAGADVNAQDLLGRTPADLAAEEGHE |
| | | | DIAEVLQKAAGSGSC |
| 19 | His Tag | | MRGSHHHHHH |
| 20 | His6-TEV | | MRGSHHHHHHENLYFQ |
| 21 | Ankyrin | Non-binding | DLGKKLLEAARAGQDDEVRELLKAGADVNAKD |
| | repeat | control | KDGYTPLHLAAREGHLEIVEVLLKAGADVNAKD |
| | domain | | KDGYTPLHLAAREGHLEIVEVLLKAGADVNAQD |
| | | | KSGKTPADLAADAGHEDIAEVLQKAA |
| 22 | Ankyrin | Non-binding | DLGKKLLEAARAGQDDEVRELLKAGADVNAKD |
| | repeat protein | control | KDGYTPLHLAAREGHLEIVEVLLKAGADVNAKD |
| | | | KDGYTPLHLAAREGHLEIVEVLLKAGADVNAQD |
| | | | KSGKTPADLAADAGHEDIAEVLQKAAGSPTPTP |
| | | | TTPTPTPTTPTPTPTGSDLGKKLLEAARAGQDD |
| | | | EVRELLKAGADVNAKDKDGYTPLHLAAREGHL |
| | | | EIVEVLLKAGADVNAKDKDGYTPLHLAAREGHL |
| | | | EIVEVLLKAGADVNAQDKSGKTPADLAADAGHE |
| | | | DIAEVLQKAA |
| 23 | N-terminal | | DLGKKLLEAARAGQDDEVRELLKAGADVNA |
| | capping | | |
| | module | | |
| 24 | Internal | | KDAAGETPLHLAAWFGHLEIVEVLLKAGADVNA |
| | repeat | | |
| | module | | |
| 25 | C-terminal | | QDLQGKTPADLAAKEGHEDIAEVLQKAA |
| | capping | | |
| | module | | |
| 26 | Ankyrin | Control | GSDLGKKLLEAARAGQDDEVRELLKAGADVNA |
| | repeat protein | | KDYFSHTPLHLAARNGHLKIVEVLLKAGADVNA |
| | | | KDFAGKTPLHLAADAGHLEIVEVLLKAGADVNA |
| | | | QDIFGKTPADIAADAGHEDIAEVLQKAAGSPTPT |
| | | | PTTPTPTPTTPTPTPTGSDLGKKLLEAARAGQD |
| | | | DEVRELLKAGADVNAKDKDGYTPLHLAAREGH |
| | | | LEIVEVLLKAGADVNAKDKDGYTPLHLAAREGH |
| | | | LEIVEVLLKAGADVNAQDKSGKTPADLAADAGH |
| | | | EDIAEVLQKAAGSGSC |
| 27 | Human DLL3 | | MVSPRMSGLLSQTVILALIFLPQTRPAGVFELQI |
| | | | HSFGPGPGPGAPRSPCSARLPCRLFFRVCLKP |
| | | | GLSEEAAESPCALGAALSARGPVYTEQPGAPA |
| | | | PDLPLPDGLLQVPFRDAWPGTFSFIIETWREEL |
| | | | GDQIGGPAWSLLARVAGRRRLAAGGPWARDI |
| | | | QRAGAWELRFSYRARCEPPAVGTACTRLCRP |
| | | | RSAPSRCGPGLRPCAPLEDECEAPLVCRAGCS |
| | | | PEHGFCEQPGECRCLEGWTGPLCTVPVSTSS |
| | | | CLSPRGPSSATTGCLVPGPGPCDGNPCANGG |
| | | | SCSETPRSFECTCPRGFYGLRCEVSGVTCADG |
| | | | PCFNGGLCVGGADPDSAYICHCPPGFQGSNC |
| | | | EKRVDRCSLQPCRNGGLCLDLGHALRCRCRA |
| | | | GFAGPRCEHDLDDCAGRACANGGTCVEGGGA |
| | | | HRCSCALGFGGRDCRERADPCAARPCAHGGR |
| | | | CYAHFSGLVCACAPGYMGARCEFPVHPDGAS |
| | | | ALPAAPPGLRPGDPQRYLLPPALGLLVAAGVA |
| | | | GAALLLVHVRRRGHSQDAGSRLLAGTPEPSVH |
| | | | ALPDALNNLRTQEGSGDGPSSSVDWNRPEDV |
| | | | DPQGIYVISAPSIYAREVATPLFPPLHTGRAGQR |
| | | | QHLLFPYPSSILSVK |
| 28 | Human | | MKWVTFISLLFLFSSAYSRGVFRRDAHKSEVAH |
| | serum | | RFKDLGEENFKALVLIAFAQYLQQCPFEDHVKL |
| | albumin | | VNEVTEFAKTCVADESAENCDKSLHTLFGDKL |
| | | | CTVATLRETYGEMADCCAKQEPERNECFLQHK |
| | | | DDNPNLPRLVRPEVDVMCTAFHDNEETFLKKY |
| | | | LYEIARRHPYFYAPELLFFAKRYKAAFTECCQA |
| | | | ADKAACLLPKLDELRDEGKASSAKQRLKCASL |
| | | | QKFGERAFKAWAVARLSQRFPKAEFAEVSKLV |
| | | | TDLTKVHTECCHGDLLECADDRADLAKYICENQ |
| | | | DSISSKLKECCEKPLLEKSHCIAEVENDEMPAD |
| | | | LPSLAADFVESKDVCKNYAEAKDVFLGMFLYEY |
| | | | ARRHPDYSWVLLLRLAKTYETTLEKCCAAADPH |
| | | | ECYAKVFDEFKPLVEEPQNLIKQNCELFEQLGE |
| | | | YKFQNALLVRYTKKVPQVSTPTLVEVSRNLGKV |
| | | | GSKCCKHPEAKRMPCAEDYLSVVLNQLCVLHE |
| | | | KTPVSDRVTKCCTESLVNRRPCFSALEVDETY |
| | | | VPKEFNAETFTFHADICTLSEKERQIKKQTALVE |
| | | | LVKHKPKATKEQLKAVMDDFAAFVEKCCKADD |
| | | | KETCFAEEGKKLVAASQAALGL |

1-17. (canceled)

18. A conjugate or pharmaceutically acceptable salt thereof, the conjugate comprising (i) an ankyrin repeat domain with binding specificity for DLL3, (ii) a chelator, and (iii) a radionuclide, wherein said chelator is covalently connected to said ankyrin repeat domain with binding specificity for DLL3, wherein said radionuclide is bound to said chelator, and wherein said radionuclide is Pb-212 or Pb-203.

19. The conjugate or pharmaceutically acceptable salt of claim 18, wherein said ankyrin repeat domain with binding specificity for DLL3 binds human DLL3 with a K_{D} value of 10 nM or below.

20. The conjugate or pharmaceutically acceptable salt of claim 18, wherein said ankyrin repeat domain with binding specificity for DLL3 comprises an amino acid sequence that is at least 80% identical to any one of SEQ ID NOs: 1 to 4.

21. The conjugate or pharmaceutically acceptable salt of claim 18, wherein said chelator is DOTA (1,4,7,10-tetraazacyclododecane-1,4,7,10-tetraacetic acid) or TCMC (1,4,7,10-Tetraazacyclododecane-1,4,7,10-tetraacetamide), or a derivative thereof.

22. The conjugate or pharmaceutically acceptable salt of claim 18, wherein said chelator has a structure of Formula (I): 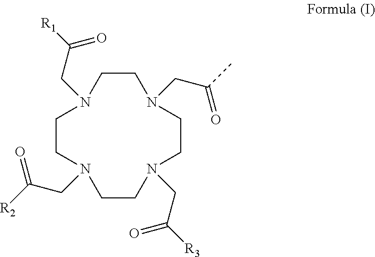
wherein R1, R2 and R3 are independently NH₂ or OH, and wherein the dotted line represents the covalent connection to said ankyrin repeat domain with binding specificity for DLL3;
or wherein said chelator has a structure of Formula (III): 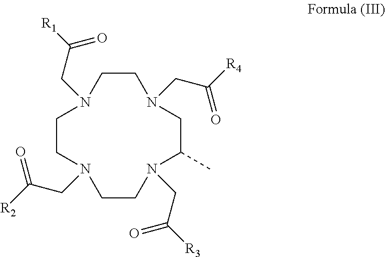
wherein R1, R2, R3 and R4 are independently NH₂ or OH, and wherein the dotted line represents the covalent connection to said ankyrin repeat domain with binding specificity for DLL3.

23. The conjugate or pharmaceutically acceptable salt of claim 18, further comprising a tag, wherein said tag comprises a cysteine.

24. The conjugate or pharmaceutically acceptable salt of claim 18, further comprising a connector, wherein said connector is covalently connected to said ankyrin repeat domain with binding specificity for DLL3 and to said chelator, and wherein said conjugate has the formula: D-Co-Ch-R, wherein D is said ankyrin repeat domain with binding specificity for DLL3, Co is said connector, Ch is said chelator, and R is said radionuclide.

25. The conjugate or pharmaceutically acceptable salt of claim 24, wherein said connector comprises a maleimide or a derivative thereof.

26. The conjugate or pharmaceutically acceptable salt of claim 18, further comprising a half-life extending moiety.

27. The conjugate or pharmaceutically acceptable salt of claim 26, wherein said half-life extending moiety is an ankyrin repeat domain with binding specificity for human serum albumin.

28. The conjugate or pharmaceutically acceptable salt of claim 27, wherein said ankyrin repeat domain with binding specificity for human serum albumin comprises an amino acid sequence that is at least 80% identical to any one of SEQ ID NOs: 5 to 7.

29. The conjugate or pharmaceutically acceptable salt of claim 18, further comprising a half-life extending moiety, a tag, and a connector, wherein said half-life extending moiety is an ankyrin repeat domain with binding specificity for human serum albumin, wherein said tag comprises a Cysteine, wherein said connector comprises a maleimide or a derivative thereof, and wherein said conjugate has the formula: H-D-T-Co-Ch-R, wherein H is said half-life extending moiety, D is said ankyrin repeat domain with binding specificity for DLL3, T is said tag, Co is said connector, Ch is said chelator, and R is said radionuclide.

30. The conjugate or pharmaceutically acceptable salt of claim 18, wherein said conjugate comprises an amino acid sequence that is at least 80% identical to any one of SEQ ID NOs: 11 to 14.

31. A conjugate or pharmaceutically acceptable salt thereof, the conjugate comprising (i) an ankyrin repeat protein comprising an amino acid sequence that is at least 80% identical to any one of SEQ ID NOs: 15 to 18, (ii) a chelator, and (iii) a radionuclide, wherein said ankyrin repeat protein has binding specificity for DLL3 and for human serum albumin, wherein said chelator is covalently connected to said ankyrin repeat protein, wherein said radionuclide is bound to said chelator, and wherein said radionuclide is Pb-212 or Pb-203.

32. A conjugate or pharmaceutically acceptable salt thereof, wherein said conjugate has a structure of Formula (VI): 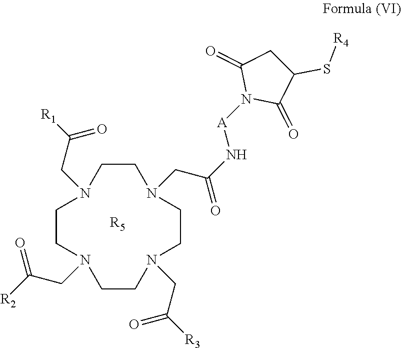
wherein R1, R2, and R3 are independently NH₂ or OH;
wherein A is CₐH_{b}N_{c}Oₐ, wherein a, b, c, and d are integers;
wherein R4 is an ankyrin repeat protein comprising an amino acid sequence that is at least 80% identical to any one of SEQ ID NOs: 15 to 18, wherein said ankyrin repeat protein has binding specificity for DLL3 and for human serum albumin, and wherein said ankyrin repeat protein comprises a Cysteine;
and wherein R5 is a chelated radionuclide, wherein said radionuclide is Pb-212 or Pb-203;
or wherein said conjugate has a structure of Formula (VII): 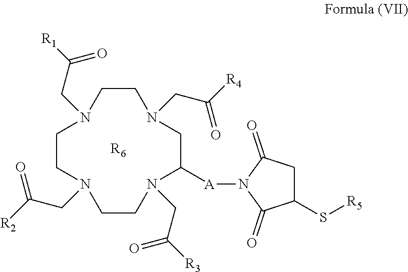
wherein R1, R2, R3 and R4 are independently NH₂ or OH;
wherein A is CₐH_{b}N_{c}Oₐ, wherein a, b, c, and d are integers;
wherein R5 is an ankyrin repeat protein comprising an amino acid sequence that is at least 80% identical to any one of SEQ ID NOs: 15 to 18, wherein said ankyrin repeat protein has binding specificity for DLL3 and for human serum albumin, and wherein said ankyrin repeat protein comprises a Cysteine;
and wherein R6 is a chelated radionuclide, wherein said radionuclide is Pb-212 or Pb-203.

33. The conjugate or pharmaceutically acceptable salt of claim 32, wherein all three of R1, R2 and R3 in Formula (VI) or all four of R1, R2, R3 and R4 in Formula (VII) are NH₂.

34. The conjugate or pharmaceutically acceptable salt of claim 32, wherein the A in Formula (VI) or the A in Formula (VII) is—CH2-CH2-.

35. A pharmaceutical composition comprising the conjugate or pharmaceutically acceptable salt of claim 18, and optionally a pharmaceutically acceptable carrier or excipient.

36. A method of treating a medical condition, the method comprising the step of administering to a subject in need thereof a therapeutically effective amount of the conjugate or pharmaceutically acceptable salt of claim 18, wherein said radionuclide is Pb-212.

37. A method of imaging and/or diagnosing a medical condition, the method comprising the steps of: (i) administering to a subject an amount of the conjugate or pharmaceutically acceptable salt of claim 18, effective for binding of the conjugate or pharmaceutically acceptable salt to cells expressing DLL3 on their surface, and (ii) detecting cells bound by the conjugate or pharmaceutically acceptable salt thereof or tissues comprising cells bound by the conjugate or pharmaceutically acceptable salt thereof.


SEQUENCE LISTING (28 sequences)

SEQ ID NO: 1 - protein, 124 aa, synthetic construct
  source 1..124: note = Ankyrin repeat domain specific for DLL3 (MAM093)
DLGKKLLAAAAQGQDDEVRELLKAGANVNAKDLFGYTPLHLAAWRGHLEIVEVLLKAGADVNAKDAAGETPLHLAAWFGHLEIVEVLLKAGADVNAQDLQGKTPADLAAKEGHEDIAEVLQKAA

SEQ ID NO: 2 - protein, 124 aa, synthetic construct
  source 1..124: note = Ankyrin repeat domain specific for DLL3 (MAM088)
DLGKKLLAAAREGQDDEVRELLKAGADVNAKDIWGQTPLHIAAWFGHLEIVEVLLKAGADVNAKDQTGHTPLHLAAKFGHLEIVEVLLKAGADVNAQDLLGRTPADLAAEEGHEDIAEVLQKAA

SEQ ID NO: 3 - protein, 124 aa, synthetic construct
  source 1..124: note = Ankyrin repeat domain specific for DLL3 (MAM112)
DLGKKLLAAAADGQDDEVRELLQAGADVNAKDLFGYTPLHLAAWRGHLEIVEVLLEAGADVNAKDAAGETPLHLAAWFGHLEIVEVLLEAGADVNAQDVQGKTPADLAAKEGHEDIAEVLQQAA

SEQ ID NO: 4 - protein, 124 aa, synthetic construct
  source 1..124: note = Ankyrin repeat domain specific for DLL3 (MAM120)
DLGTKLLSAAADGQDDEVRELLQAGADVNAKDLFGYTPLHLAAWRGHLEIVEVLLEAGADVNAKDAAGLTPLHLAAWFGHLEIVEVLLEAGADVNAQDVQGVTPADLAAKEGHEDIAEVLQQAA

SEQ ID NO: 5 - protein, 124 aa, synthetic construct
  source 1..124: note = Ankyrin repeat domain specific for human serum albumin
DLGKKLLEAARAGQDDEVRELLKAGADVNAKDYFSHTPLHLAARNGHLKIVEVLLKAGADVNAKDFAGKTPLHLAANEGHLEIVEVLLKAGADVNAQDIFGKTPADIAADAGHEDIAEVLQKAA

SEQ ID NO: 6 - protein, 124 aa, synthetic construct
  source 1..124: note = Ankyrin repeat domain specific for human serum albumin
DLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAA

SEQ ID NO: 7 - protein, 124 aa, synthetic construct
  source 1..124: note = Ankyrin repeat domain specific for human serum albumin
DLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAA

SEQ ID NO: 8 - protein, 24 aa, synthetic construct
  source 1..24: note = PT linker
GSPTPTPTTPTPTPTTPTPTPTGS

SEQ ID NO: 9 - protein, 5 aa, synthetic construct
  source 1..5: note = Consensus GS linker
  REGION 1..>5: note = The entire sequence of amino acids 1-5 can be repeated 1, 2, 3, 4, 5 or 6 times.
GGGGS

SEQ ID NO: 10 - protein, 5 aa, synthetic construct
  source 1..5: note = Tag
GSGSC

SEQ ID NO: 11 - protein, 272 aa, synthetic construct
  source 1..272: note = Ankyrin repeat protein (MAM279)
DLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAAGSPTPTPTTPTPTPTTPTPTPTGSDLGKKLLAAAAQGQDDEVRELLKAGANVNAKDLFGYTPLHLAAWRGHLEIVEVLLKAGADVNAKDAAGETPLHLAAWFGHLEIVEVLLKAGADVNAQDLQGKTPADLAAKEGHEDIAEVLQKAA

SEQ ID NO: 12 - protein, 272 aa, synthetic construct
  source 1..272: note = Ankyrin repeat protein (MAM283)
DLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAAGSPTPTPTTPTPTPTTPTPTPTGSDLGKKLLAAAADGQDDEVRELLQAGADVNAKDLFGYTPLHLAAWRGHLEIVEVLLEAGADVNAKDAAGETPLHLAAWFGHLEIVEVLLEAGADVNAQDVQGKTPADLAAKEGHEDIAEVLQQAA

SEQ ID NO: 13 - protein, 272 aa, synthetic construct
  source 1..272: note = Ankyrin repeat protein (MAM160)
DLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAAGSPTPTPTTPTPTPTTPTPTPTGSDLGTKLLSAAADGQDDEVRELLQAGADVNAKDLFGYTPLHLAAWRGHLEIVEVLLEAGADVNAKDAAGLTPLHLAAWFGHLEIVEVLLEAGADVNAQDVQGVTPADLAAKEGHEDIAEVLQQAA

SEQ ID NO: 14 - protein, 272 aa, synthetic construct
  source 1..272: note = Ankyrin repeat protein (MAM282)
DLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAAGSPTPTPTTPTPTPTTPTPTPTGSDLGKKLLAAAREGQDDEVRELLKAGADVNAKDIWGQTPLHIAAWFGHLEIVEVLLKAGADVNAKDQTGHTPLHLAAKFGHLEIVEVLLKAGADVNAQDLLGRTPADLAAEEGHEDIAEVLQKAA

SEQ ID NO: 15 - protein, 279 aa, synthetic construct
  source 1..279: note = Ankyrin repeat protein with tag (GS-MAM279-tag)
GSDLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAAGSPTPTPTTPTPTPTTPTPTPTGSDLGKKLLAAAAQGQDDEVRELLKAGANVNAKDLFGYTPLHLAAWRGHLEIVEVLLKAGADVNAKDAAGETPLHLAAWFGHLEIVEVLLKAGADVNAQDLQGKTPADLAAKEGHEDIAEVLQKAAGSGSC

SEQ ID NO: 16 - protein, 279 aa, synthetic construct
  source 1..279: note = Ankyrin repeat protein with tag (GS-MAM283-tag)
GSDLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAAGSPTPTPTTPTPTPTTPTPTPTGSDLGKKLLAAAADGQDDEVRELLQAGADVNAKDLFGYTPLHLAAWRGHLEIVEVLLEAGADVNAKDAAGETPLHLAAWFGHLEIVEVLLEAGADVNAQDVQGKTPADLAAKEGHEDIAEVLQQAAGSGSC

SEQ ID NO: 17 - protein, 279 aa, synthetic construct
  source 1..279: note = Ankyrin repeat protein with tag (GS-MAM160-tag)
GSDLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAAGSPTPTPTTPTPTPTTPTPTPTGSDLGTKLLSAAADGQDDEVRELLQAGADVNAKDLFGYTPLHLAAWRGHLEIVEVLLEAGADVNAKDAAGLTPLHLAAWFGHLEIVEVLLEAGADVNAQDVQGVTPADLAAKEGHEDIAEVLQQAAGSGSC

SEQ ID NO: 18 - protein, 279 aa, synthetic construct
  source 1..279: note = Ankyrin repeat protein with tag (GS-MAM282-tag)
GSDLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAAGSPTPTPTTPTPTPTTPTPTPTGSDLGKKLLAAAREGQDDEVRELLKAGADVNAKDIWGQTPLHIAAWFGHLEIVEVLLKAGADVNAKDQTGHTPLHLAAKFGHLEIVEVLLKAGADVNAQDLLGRTPADLAAEEGHEDIAEVLQKAAGSGSC

SEQ ID NO: 19 - protein, 10 aa, synthetic construct
  source 1..10: note = His Tag
MRGSHHHHHH

SEQ ID NO: 20 - protein, 16 aa, synthetic construct
  source 1..16: note = His6-TEV
MRGSHHHHHHENLYFQ

SEQ ID NO: 21 - protein, 124 aa, synthetic construct
  source 1..124: note = Ankyrin repeat domain (non-binding control)
DLGKKLLEAARAGQDDEVRELLKAGADVNAKDKDGYTPLHLAAREGHLEIVEVLLKAGADVNAKDKDGYTPLHLAAREGHLEIVEVLLKAGADVNAQDKSGKTPADLAADAGHEDIAEVLQKAA

SEQ ID NO: 22 - protein, 272 aa, synthetic construct
  source 1..272: note = Ankyrin repeat protein (non-binding control)
DLGKKLLEAARAGQDDEVRELLKAGADVNAKDKDGYTPLHLAAREGHLEIVEVLLKAGADVNAKDKDGYTPLHLAAREGHLEIVEVLLKAGADVNAQDKSGKTPADLAADAGHEDIAEVLQKAAGSPTPTPTTPTPTPTTPTPTPTGSDLGKKLLEAARAGQDDEVRELLKAGADVNAKDKDGYTPLHLAAREGHLEIVEVLLKAGADVNAKDKDGYTPLHLAAREGHLEIVEVLLKAGADVNAQDKSGKTPADLAADAGHEDIAEVLQKAA

SEQ ID NO: 23 - protein, 30 aa, synthetic construct
  source 1..30: note = N-terminal capping module
DLGKKLLEAARAGQDDEVRELLKAGADVNA

SEQ ID NO: 24 - protein, 33 aa, synthetic construct
  source 1..33: note = Internal repeat module
KDAAGETPLHLAAWFGHLEIVEVLLKAGADVNA

SEQ ID NO: 25 - protein, 28 aa, synthetic construct
  source 1..28: note = C-terminal capping module
QDLQGKTPADLAAKEGHEDIAEVLQKAA

SEQ ID NO: 26 - protein, 279 aa, synthetic construct
  source 1..279: note = Ankyrin repeat protein (control)
GSDLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAAGSPTPTPTTPTPTPTTPTPTPTGSDLGKKLLEAARAGQDDEVRELLKAGADVNAKDKDGYTPLHLAAREGHLEIVEVLLKAGADVNAKDKDGYTPLHLAAREGHLEIVEVLLKAGADVNAQDKSGKTPADLAADAGHEDIAEVLQKAAGSGSC

SEQ ID NO: 27 - protein, 618 aa, Homo sapiens
  source 1..618: note = Human DLL3
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 28 - protein, 609 aa, Homo sapiens [sequence omitted]